SEQUENCE LISTING (50 sequences)

SEQ ID NO: 1 - DNA, 20 bp, Homo sapiens
  source 1..20: mol_type = other DNA
agagggaaatcgtgcgtgac

SEQ ID NO: 2 - DNA, 19 bp, Homo sapiens
  source 1..19: mol_type = other DNA
aggagccagggcagtaatc

SEQ ID NO: 3 - DNA, 19 bp, Homo sapiens
  source 1..19: mol_type = other DNA
ctccgtggccttagctgtg

SEQ ID NO: 4 - DNA, 22 bp, Homo sapiens
  source 1..22: mol_type = other DNA
tttggagtacgctggatagcct

SEQ ID NO: 5 - DNA, 20 bp, Homo sapiens
  source 1..20: mol_type = other DNA
tgtggctttaggagcagtgg

SEQ ID NO: 6 - DNA, 20 bp, Homo sapiens
  source 1..20: mol_type = other DNA
gctacaggcaggtcagtgag

SEQ ID NO: 7 - DNA, 19 bp, Homo sapiens
  source 1..19: mol_type = other DNA
ttttaccgcccagagagcg

SEQ ID NO: 8 - DNA, 19 bp, Homo sapiens
  source 1..19: mol_type = other DNA
aatgacaaggagagccacc

SEQ ID NO: 9 - DNA, 25 bp, Homo sapiens
  source 1..25: mol_type = other DNA
tggacctaatgaattcctcagaaaa

SEQ ID NO: 10 - DNA, 23 bp, Homo sapiens
  source 1..23: mol_type = other DNA
acacagaaattagttcagcagca

SEQ ID NO: 11 - DNA, 23 bp, Homo sapiens
  source 1..23: mol_type = other DNA
ctacaagggatcgggtttatgga

SEQ ID NO: 12 - DNA, 21 bp, Homo sapiens
  source 1..21: mol_type = other DNA
ttggcattgcctagtagcgta

SEQ ID NO: 13 - DNA, 19 bp, Homo sapiens
  source 1..19: mol_type = other DNA
tcaatgccccatacaacaa

SEQ ID NO: 14 - DNA, 20 bp, Homo sapiens
  source 1..20: mol_type = other DNA
tgcttgtccagatgacttcg

SEQ ID NO: 15 - DNA, 23 bp, Homo sapiens
  source 1..23: mol_type = other DNA
ctgctcatctatacacggttacc

SEQ ID NO: 16 - DNA, 22 bp, Homo sapiens
  source 1..22: mol_type = other DNA
ggaaacgtcgtacagttctgtg

SEQ ID NO: 17 - DNA, 24 bp, Homo sapiens
  source 1..24: mol_type = other DNA
ccatcagcttgtctgtttcattcc

SEQ ID NO: 18 - DNA, 23 bp, Homo sapiens
  source 1..23: mol_type = other DNA
gctgtaatccaaggaatgtggtc

SEQ ID NO: 19 - DNA, 22 bp, Homo sapiens
  source 1..22: mol_type = other DNA
tggttttaatgaagcttgccag

SEQ ID NO: 20 - DNA, 22 bp, Homo sapiens
  source 1..22: mol_type = other DNA
gagatgtgccactcgtaatagg

SEQ ID NO: 21 - DNA, 20 bp, Homo sapiens
  source 1..20: mol_type = other DNA
aagcactgggagtttgatgc

SEQ ID NO: 22 - DNA, 20 bp, Homo sapiens
  source 1..20: mol_type = other DNA
attgcttttgcgcaatccct

SEQ ID NO: 23 - DNA, 21 bp, Homo sapiens
  source 1..21: mol_type = other DNA
aagactgcgttcctgctcaac

SEQ ID NO: 24 - DNA, 21 bp, Homo sapiens
  source 1..21: mol_type = other DNA
aaagccctacagcaactgtcg

SEQ ID NO: 25 - DNA, 21 bp, Homo sapiens
  source 1..21: mol_type = other DNA
gcccttcaagtgtttcaccaa

SEQ ID NO: 26 - DNA, 22 bp, Homo sapiens
  source 1..22: mol_type = other DNA
ccagccagacaaatatatgcga

SEQ ID NO: 27 - DNA, 20 bp, Homo sapiens
  source 1..20: mol_type = other DNA
cgagatgccttcagcagagt

SEQ ID NO: 28 - DNA, 20 bp, Homo sapiens
  source 1..20: mol_type = other DNA
cgccttgatgtctgggtctt

SEQ ID NO: 29 - DNA, 21 bp, Homo sapiens
  source 1..21: mol_type = other DNA
cctccactgtgctggttttat

SEQ ID NO: 30 - DNA, 20 bp, Homo sapiens
  source 1..20: mol_type = other DNA
tcagcaacatgctccagaag

SEQ ID NO: 31 - DNA, 20 bp, Homo sapiens
  source 1..20: mol_type = other DNA
gtacctgtcctgcgtgttga

SEQ ID NO: 32 - DNA, 20 bp, Homo sapiens
  source 1..20: mol_type = other DNA
gggaactgggcagactcaaa

SEQ ID NO: 33 - DNA, 20 bp, Homo sapiens
  source 1..20: mol_type = other DNA
agcatgaggctcaatgggta

SEQ ID NO: 34 - DNA, 20 bp, Homo sapiens
  source 1..20: mol_type = other DNA
aaatccagcaagatgcaagc

SEQ ID NO: 35 - DNA, 19 bp, Homo sapiens
  source 1..19: mol_type = other DNA
aggccccatgtgttcatgc

SEQ ID NO: 36 - DNA, 22 bp, Homo sapiens
  source 1..22: mol_type = other DNA
gcgcaaagtagtcagtccatct

SEQ ID NO: 37 - DNA, 20 bp, Homo sapiens
  source 1..20: mol_type = other DNA
ataagcgagggccagctttc

SEQ ID NO: 38 - DNA, 20 bp, Homo sapiens
  source 1..20: mol_type = other DNA
cgcagtttacgctgtctagc

SEQ ID NO: 39 - DNA, 20 bp, Homo sapiens
  source 1..20: mol_type = other DNA
tgcgacaaaacctcctcctt

SEQ ID NO: 40 - DNA, 21 bp, Homo sapiens
  source 1..21: mol_type = other DNA
tgttgatgccttcacagcgta

SEQ ID NO: 41 - DNA, 22 bp, Homo sapiens
  source 1..22: mol_type = other DNA
gaaccatgtgccactggaaggc

SEQ ID NO: 42 - DNA, 22 bp, Homo sapiens
  source 1..22: mol_type = other DNA
agcggaatctcctggtgcagtt

SEQ ID NO: 43 - DNA, 20 bp, Homo sapiens
  source 1..20: mol_type = other DNA
agcgcaagggtttctggttt

SEQ ID NO: 44 - DNA, 20 bp, Homo sapiens
  source 1..20: mol_type = other DNA
aataggctgtggggtcagtc

SEQ ID NO: 45 - DNA, 20 bp, Homo sapiens
  source 1..20: mol_type = other DNA
agcacggtctggcaaataca

SEQ ID NO: 46 - DNA, 20 bp, Homo sapiens
  source 1..20: mol_type = other DNA
atccatccagcagcacagac

SEQ ID NO: 47 - DNA, 25 bp, Homo sapiens
  source 1..25: mol_type = other DNA
agttgcagctcggagttctgagaca

SEQ ID NO: 48 - DNA, 22 bp, Homo sapiens
  source 1..22: mol_type = other DNA
gaaccatgtgccactggaaggc

SEQ ID NO: 49 - DNA, 20 bp, Homo sapiens
  source 1..20: mol_type = other DNA
ctacctccaccatgccaagt

SEQ ID NO: 50 - DNA, 20 bp, Homo sapiens
  source 1..20: mol_type = other DNA
agctgcgctgatagacatcc

(19) United States
(12) Patent Application Publication
(10) Pub. No.: US 2024/0182966 A1
(43) Pub. Date: Jun. 6, 2024

(54) CRITICAL QUALITY ATTRIBUTES FOR IDENTIFYING MESENCHYMAL STROMAL CELLS WITH IMMUNOMODULATORY AND ANGIOGENIC FITNESS

(71) Applicant: University Health Network, Toronto (CA)

(72) Inventors: Sowmya Viswanathan, Toronto (CA); Kevin Robb, Toronto (CA)

(21) Appl. No.: 18/210,490

(22) Filed: Jun. 15, 2023

Related U.S. Application Data
(60) Provisional application No. 63/428,499, filed on Nov. 29, 2022.

(51) Int. Cl. C12Q 1/6876; C12N 5/0775

(52) U.S. Cl. CPC C12Q 1/6876; C12N 5/0663; C12Q 2600/158

(57) ABSTRACT

The present disclosure provides critical quality attributes for assessing immunomodulatory fitness or angiogenic fitness of mesenchymal stromal cells. Also provided is use of these critical quality attributes to determine quantitative values for assessing the fitness of a sample from a particular donor and/or grown using particular critical processing parameters.

### RELATED APPLICATIONS

This application claims the benefit of U.S. 63/428,499 filed on Nov. 29, 2022, herein incorporated by reference.

### INCORPORATION OF SEQUENCE LISTING

A computer readable form of the Sequence Listing “10723-P70044US01_SequenceListing.xml” (44,833 bytes), submitted via Patent Center and created on Jun. 12, 2023, is herein incorporated by reference.

### FIELD

The present disclosure relates to methods of assessing immunomodulatory or angiogenic fitness of mesenchymal stromal cells.

### INTRODUCTION

The immunomodulatory and pro-angiogenic functions of mesenchymal stromal cells (MSCs) make them attractive cell therapy candidates for numerous clinical indications [1]. However, despite hundreds of clinical trials, mixed reports on clinical efficacy and insufficient characterization of MSC potency have continued to hamper the field, resulting in very few MSC products with regulatory and market endorsement [2]. As was recently outlined by Krampera and Le Blanc [3], part of the observed heterogeneity in clinical efficacy of MSCs is likely due to the complex interactions between MSCs and the host tissue microenvironment, which is specific to a given disease as well as stage of the disease. This has further pointed to the need for defining critical quality attributes (CQAs) with established limits or ranges that enable quality checks of basal thresholds for high therapeutic potency MSCs with associated fitness levels [4]. These fitness levels may be further modulated by the host disease microenvironment to ultimately determine net MSC therapeutic efficacy [3,5], but quantifying basal MSC fitness through CQAs is a necessary starting point. Importantly, MSC CQAs must be linked to specific culture conditions, which are a critical source of variability in processing and expanding MSCs [6,7].

Previous work has investigated MSC characteristics that carry functional significance and can serve as candidate CQAs for defining basal MSC fitness levels that correlate with clinical efficacy. It has demonstrated that prevalence of a panel of seven immunomodulatory markers expressed by bone marrow-derived MSCs (MSC(M)) in vitro (i.e., basal CQAs) correlated with improved patient-reported outcomes, suggestive of an anti-inflammatory mechanism of action in a twelve-patient knee osteoarthritis trial [8]. Work by Galleu et al. has suggested that MSC(M) that are more susceptive to host cytotoxic activity afforded better clinical responses in graft-versus-host disease (GVHD) patients [9]. Several groups have evaluated multi-dimensional characteristics of MSCs that could be considered putative CQAs, as these correlate to in vitro immunosuppression of T cell functions. However, the associations between this immunomodulatory ability and clinical efficacy, at least in GVHD patients, has not panned out [10]. Nonetheless, work by Chinnadurai et al. demonstrated that interactions with peripheral blood-derived mononuclear cells (PBMCs) modulate the mRNA and secreted factor profiles [11,12], as well as the signal transducer and activator of transcription (STAT) phosphorylation status [13] of human MSCs derived from various tissue sources, and that these signatures correlate with T cell immunosuppression. Maughon et al. have shown that metabolomic and cytokine profiles of human MSC(M) and induced pluripotent stem cell-derived MSCs also correlate to T cell suppression [14]. Furthermore, multidimensional profiles of human MSC(M) morphological features have been linked to T cell suppression for MSC(M) stimulated with TNFα and/or IFNγ [15, 16]. Notably, work by Boregowda et al. postulated a potential interplay between immunomodulatory and pro-angiogenic functions of human MSC(M) mediated by expression levels of the transcription factor TWIST1, and suggested that culture conditions impact the interplay between these two properties of MSCs [17].

While progress has been made in identifying candidate CQAs for MSCs [18,19], it is important for these candidate CQAs to be measurable, quantitative and sensitive to donor heterogeneity and variations in critical processing parameters (CPPs, i.e., culture parameters that influence CQAs), two major, controllable variables that impact MSC functional activity in vitro. Subsequent in vivo MSC functionality is less tractable and likely modulated by host immune cell and microenvironment interactions. Donor heterogeneity can be attributed to several factors, including donor health status, BMI, sex, and age which are known to influence MSC functional properties, such as in vitro clonogenic potential and paracrine functions [20-22]. In addition, manufacturing strategies for MSCs vary widely, and culture conditions or CPPs can have a marked impact on cell behaviour [23,24]. For example, stimulation with pro-inflammatory cytokines, commonly referred to as “licensing” in the MSC field, has been extensively explored as a means to enhance immunoregulatory functions of MSCs and reduce donor heterogeneity [25,26].

The effects of hypoxic conditions on MSC function have been widely investigated, in particular for augmenting the pro-angiogenic functions of MSCs within in vitro and in vivo models [27,28]. Recent evidence has also shown that hypoxic culture may augment MSC immunomodulatory functions [29,30]. In parallel, 3D cultures of MSCs were considered as work by Bartosh et al. demonstrated that human MSC(M) spheroids had improved immunomodulatory functions within an in vitro mouse macrophage co-culture system and in the zymosan-induced peritonitis mouse model [31]. Other studies have provided further evidence for the augmented immunomodulatory [32,33], as well as pro-angiogenic [32,34,35] functions of MSCs cultured in 3D aggregates. Previous work has suggested that 3D-cultured MSCs lose their augmented immunomodulatory function when cultured using xeno-free medium [36].

### SUMMARY

In terms of CPPs, the present inventors elected to focus on and compare MSCs cultured under hypoxic or 3D aggregate conditions as non-genetic, culture manipulating methods that are known to augment immunomodulatory and/or angiogenic properties of MSCs, rather than traditional parameters (medium, seeding density, etc.).

The present inventors explored the relationship between select CPPs known to enhance immunomodulatory and/or angiogenic MSC basal fitness range and multivariate morphological, gene expression, soluble factor expression, functional readouts, and clinical evaluation against a backdrop of donor heterogeneity. Using adipose tissue-derived MSCs (MSC(AT)) and bone marrow derived MSCs (MSC(M)), a statistical approach was employed to identify a putative matrix of CQAs that are correlated with anchor functional assays and/or clinical efficacy. The present inventors focused on in vitro monocyte/macrophage (M) polarization and clinical evaluation, recognizing that Mos are a primary effector cell type of MSCs for numerous indications (reviewed in [37]), and given prior clinical data demonstrating that MSC(M) injections modulate Mo phenotype in knee osteoarthritis [8]. To evaluate functional angiogenesis, the human umbilical vein endothelial cell (HUVEC) tube formation assay was selected as a potency assay that has been employed for clinical-grade MSC products [19,38].

The present inventors integrated statistical methods to identify a matrix of putative CQAs with minimum and maximum values that correlated with functional immunomodulatory and/or angiogenic readouts. This combinatorial assay matrix approach allowed to systematically compare and rank the effects of varying CPPs on putative CQAs for MSC(AT) in terms of their immunomodulatory and angiogenic properties, two functional axes with high therapeutic relevance. The matrix of putative CQAs also allows for identification of donors with enhanced immunomodulation or angiogenic functionalities.

Accordingly, provided herein is a method of determining immunomodulatory fitness of mesenchymal stromal cells (MSC) comprising:
determining a sample critical quality attribute (CQA) profile, the sample CQA profile comprising:
a) from the sample harvested:
   a. in the presence of proinflammatory cytokines stimulation post-harvest: the mRNA levels of the following genes: THBS1, CCN2, EDN1, ACTA2, PDCD1LG2, TNFAIP6, ANGPT1, CXCL8; and/or
   b. in the absence of the proinflammatory cytokines stimulation post-harvest: the mRNA levels of the following genes: CCN2, TSG101, THBS1, PDGFA, VEGFA, EDIL3, ACTA2, ANGPT1, ANG and the level of TGF-beta protein; and/or
b) from a single cell suspension of the sample post-harvest without pro-inflammatory cytokines stimulation: cell diameter and cell circularity;
determining the level of similarity of said sample CQA profile to one or more control profiles, wherein (i) a high level of similarity of the sample CQA profile to an immunomodulatory specific control profile; (ii) a low level of similarity to a non-immunomodulatory specific control profile; and/or (iii) a higher level of similarity to an immunomodulatory specific control profile than to a non-immunomodulatory specific control profile indicates the cells have basal immunomodulatory fitness.

Also provided herein is a method of determining angiogenic fitness of derived mesenchymal stromal cells (MSC) comprising:
determining a sample critical quality attribute (CQA) profile, the sample CQA profile comprising:
a) from the sample harvested and cultured:
   a. in the presence of proinflammatory cytokines stimulation post-harvest: the mRNA levels of the following genes: SMAD7, HGF, ANG, IDO1, TSG101, CXCL8 and the level of VEGF protein; and/or
   b. in the absence of the proinflammatory cytokines stimulation post-harvest: the mRNA levels of the following genes: SOX9, TNFAIP6 and the level of VEGF protein;
determining the level of similarity of said sample CQA profile to one or more control profiles, wherein (i) a high level of similarity of the sample CQA profile to an angiogenic specific control profile; (ii) a low level of similarity to a non-angiogenic specific control profile; and/or (iii) a higher level of similarity to an angiogenic specific control profile than to a non-angiogenic specific control profile indicates the cells have basal angiogenic fitness.

In an embodiment, the CQA profiles are analyzed using combinatorial or multivariate dimension reduction statistical or mathematical methods. The combinatorial or multivariate dimension reduction statistical or mathematical methods optionally comprise clustering analyses or desirability analysis to enable comparisons between multivariate CQA profiles with dimension-reduced empirical values.

In an embodiment, a higher level of similarity to the immunomodulatory specific control profile than to the non-immunomodulatory specific control profile is indicated by a higher correlation value computed between the sample profile and the immunomodulatory specific control profile than an equivalent correlation value computed between the sample profile and the non-immunomodulatory specific control profile, optionally wherein the correlation value is a correlation coefficient. Similarly, a higher level of similarity to the angiogenic specific control profile than to the non-angiogenic specific control profile is indicated by a higher correlation value computed between the sample profile and the angiogenic specific control profile than an equivalent correlation value computed between the sample profile and the non-angiogenic specific control profile, optionally wherein the correlation value is a correlation coefficient.

In some embodiments, the correlation coefficient is a linear correlation coefficient, optionally a Pearson correlation coefficient, or a monotonic correlation coefficient, optionally a Spearman's correlation coefficient. In some embodiments, a high level of similarity to the control profile is indicated by a Pearson correlation coefficient or a Spearman's correlation coefficient between the sample profile and the control profile having an absolute value between 0.5 to 1, optionally between 0.75 to 1, and a low level of similarity to the control profile is indicated by a correlation coefficient between the sample profile and the control profile having an absolute value between 0 to 0.5, optionally between 0 to 0.25.

Also provided herein is a method of determining quantitative values for a set of CQAs for assessing immunomodulatory fitness of samples of mesenchymal stromal cells (MSC), wherein the MSC samples are from donors and/or from samples grown using different critical processing parameters (CPPs), the method comprising:
a) determining the values of the set of CQAs for each sample; wherein the set of CQAs comprise:
   a. from the sample cultured and harvested:
      i. in the presence of proinflammatory cytokines stimulation post-harvest: the mRNA levels of the following genes: THBS1, CCN2, EDN1, ACTA2, PDCD1LG2, TNFAIP6, ANGPT1, CXCL8; and/or
      ii. in the absence of the proinflammatory cytokines stimulation post-harvest: the mRNA levels of the following genes: CCN2, TSG101, THBS1, PDGFA, VEGFA, EDIL3, ACTA2, ANGPT1, ANG and the level of TGF-beta protein; and/or
   b. from a single cell suspension of the sample: cell diameter and cell circularity post-harvest without pro-inflammatory cytokine stimulation;
b) testing each sample of MSC for immunomodulatory fitness using in vitro assays or obtaining immunomodulatory fitness status of the sample;
c) conducting a regression analysis between each CQA of a) with the immunomodulatory fitness of b); determining minimum and maximum data values of each CQA of the samples and using multivariate dimension reduction statistical or mathematical methods to assign minimization or maximization functions to indicate whether higher or lower values are desirable based on the directionality of correlation respectively.

In an embodiment, the method optionally further comprises
d) assigning a weighting to each CQA for desirability analysis based on the R² values from the regression analysis; and
e) use of desirability analysis methods to assign a score from zero to one to each sample based on the range of data values; wherein zero is undesirable and one is highly desirable to obtain a set of values for MSCs for each donor grown using particular CPPs.

In one embodiment, the testing for immunomodulatory fitness in step b) comprises measuring in vitro MSC-mediated polarization of monocyte/macrophages toward inflammation-resolving phenotypes, regulatory T cell (Treg) induction, suppression of peripheral blood mononuclear cells (PBMC) or T cell proliferation.

In another embodiment, the testing for immunomodulatory fitness in step b) comprises evaluating immunomodulatory effects of MSC using preclinical animal models.

In yet another embodiment, the immunomodulatory fitness status of the sample in step b) is obtained from a clinical sample for which the immunomodulatory fitness or efficacy of the MSCs were previously evaluated clinically.

In a further embodiment, the obtaining of the immunomodulatory fitness status of the sample in step b) comprises:
I) determining at least one patient-reported outcome measure (PROM) score from an assessment of an MSC-treatable disease from a subject with the MSC-treatable disease pre treatment;
II) determining the at least one PROM score from the assessment of the MSC-treatable disease from the subject post treatment;
III) comparing the at least one PROM score in I) and II); and
IV) assigning an immunomodulatory fitness score to the sample based on the size of the improvement in the at least one PROM score post-treatment compared to pre-treatment, wherein an improvement in the at least one PROM score post-treatment is indicative that the immunomodulatory fitness score of the sample is high, and wherein no improvement or a worsening in the at least one PROM score post-treatment is indicative that the immunomodulatory fitness score of the sample is low.

In an embodiment, the method of a) comprises a.i. and/or a.ii.

In another embodiment, step II) is performed 12 or 24 months post treatment with the sample.

In one embodiment, the MSC-treatable disease comprises osteoarthritis, lupus, scleroderma, rheumatoid arthritis, graft versus host disease, stroke, inflammatory bowel disease, or cardiac disease.

In another embodiment, the MSC-treatable disease comprises osteoarthritis.

In yet another embodiment, the osteoarthritis comprises knee osteoarthritis, and the assessment of the MSC-treatable disease comprises an assessment of knee osteoarthritis.

In one embodiment, the assessment of knee osteoarthritis comprises Knee injury and Osteoarthritis Outcome Score (KOOS), Western Ontario and McMaster Universities Osteoarthritis Index (WOMAC), Visual Analogue Scale (VAS), Short Form 36 (SF-36) or Short Form 12 (SF-12), Tegner Lysholm Knee Score, Knee Society Clinical Rating System, Lequesne Index for Knee Osteoarthritis, Oxford Knee Score (OKS) and/or International Knee Documentation Committee (IKDC) Questionnaire.

In another embodiment, the assessment of knee osteoarthritis comprises KOOS, and the PROM score comprises KOOS Pain, ADL, function in Sport and Recreation (Sports/Rec), knee-related quality of life (QOL), or Overall KOOS, or combinations thereof.

Also provided herein is a method of determining immunomodulatory fitness of mesenchymal stromal cells (MSC) comprising
a) determining a sample critical quality attribute (CQA) profile, the sample CQA profile comprising:
   a. from the sample harvested in the presence of proinflammatory cytokines stimulation post-harvest: the mRNA levels of the following genes: THBS1, CCN2, EDN1, ACTA2, PDCD1LG2, TNFAIP6, ANGPT1, CXCL8; and/or
   b. from the sample harvested in the absence of the proinflammatory cytokines stimulation post-harvest: the mRNA levels of the following genes: CCN2, TSG101, THBS1, PDGFA, VEGFA, EDIL3, ACTA2, ANGPT1, ANG and the level of TGF-beta protein; and/or
   c. from a single cell suspension of the sample post-harvest without pro-inflammatory cytokines stimulation: cell diameter and cell circularity; and
b) ranking the CQA profile of a) based on a comparison of minimum and maximum values determined by the method disclosed herein for the same particular donor using the same particular CPPs to determine the immunomodulatory fitness.

In another aspect, herein provided is a method of determining quantitative values for a set of CQAs for assessing angiogenic fitness of samples of mesenchymal stromal cells (MSC), wherein the MSC samples are from different donors and/or from samples grown using different critical processing parameters (CPPs), the method comprising:
a) determining the values of the set of CQAs for each sample; wherein the set of CQAs comprise:
   a. from the sample cultured and harvested:
      i. in the presence of proinflammatory cytokines post-harvest stimulation: the mRNA levels of the following genes: SMAD7, HGF, ANG, IDO1, TSG101, CXCL8 and the level of VEGF protein; and/or
      ii. in the absence of the proinflammatory cytokines post-harvest stimulation: the mRNA levels of the following genes: SOX9, TNFAIP6 and the level of VEGF protein; and
b) testing each sample of MSCs for angiogenic fitness using in vitro assays or obtaining angiogenic fitness status of the sample; and
c) conducting a regression analysis between each sample CQA of a) with the angiogenic fitness of b); determining minimum and maximum data values of each CQA of the samples and using multivariate dimension reduction statistical or mathematical methods to assign minimization or maximization functions to indicate whether higher or lower values are desirable based on the directionality correlation respectively.

In an embodiment, the method further comprises
d) assigning a weighting to each CQA for desirability analysis based on the R² values from the regression analysis; and
e) use of desirability analysis methods to assign a score from zero to one to each sample based on the range of data values; wherein zero is undesirable and one is highly desirable to obtain a set of values for MSCs for each donor grown using particular CPPs.

In one embodiment, the testing for angiogenic fitness in step b) comprises an in vitro human umbilical vein tube formation assay, measurement of MSC-mediated effects on endothelial cells, assays for vessel outgrowth or the chick chorioallantoic membrane assay. In another embodiment, the testing for angiogenic fitness in step b) comprises evaluating angiogenic effects of MSC using preclinical animal models. In yet another embodiment, the angiogenic fitness status of the sample in step b) is obtained from a clinical sample for which the angiogenic fitness or efficacy of the MSCs were previously evaluated clinically.

Also provided herein is a method of determining angiogenic fitness of mesenchymal stromal cells (MSC) from a particular donor using particular CPPs comprising
a) determining a sample critical quality attribute (CQA) profile, the sample CQA profile comprising:
   a. from the sample harvested in the presence of proinflammatory cytokines stimulation post-harvest: the mRNA levels of the following genes: SMAD7, HGF, ANG, IDO1, TSG101, CXCL8 and the level of VEGF protein; and/or
   b. from the sample harvested in the absence of the proinflammatory cytokines stimulation post-harvest: the mRNA levels of the following genes: SOX9, TNFAIP6 and the level of VEGF protein;
b) ranking the CQA profile of a) based on a comparison of minimum and maximum values determined by the method disclosed herein for the same particular donor using the same particular CPPs to determine the angiogenic fitness.

In an embodiment, the MSCs are derived from adipose tissue, bone marrow, dental pulp, synovium, placenta, or umbilical cord.

In an embodiment, the MSCs are derived from adipose tissue.

In an embodiment, the MSCs are derived from bone marrow.

In an embodiment, the proinflammatory cytokines stimulation post-harvest comprises IFNγ, TNFα and/or IL-1β.

The preceding section is provided by way of example only and is not intended to be limiting on the scope of the present disclosure and appended claims. Additional objects and advantages associated with the compositions and methods of the present disclosure will be appreciated by one of ordinary skill in the art in light of the instant claims, description, and examples. For example, the various aspects and embodiments of the disclosure may be utilized in numerous combinations, all of which are expressly contemplated by the present description. These additional advantages, objects and embodiments are expressly included within the scope of the present disclosure. The publications and other materials used herein to illuminate the background of the disclosure, and in particular cases, to provide additional details respecting the practice, are incorporated by reference, and for convenience are listed in the appended reference section.

### DRAWINGS

Further objects, features and advantages of the disclosure will become apparent from the following detailed description taken in conjunction with the accompanying figures showing illustrative embodiments of the disclosure, in which:

FIG. 1 provides a schematic of experimental and statistical analyses to identify a putative matrix of critical quality attributes sensitive to changes in specific critical processing parameters that enhance MSC(AT) potency in an exemplary embodiment of the disclosure. The overarching aim was to evaluate the response of putative critical quality attributes (CQAs) to variations in culture conditions, critical processing parameters (CPPs) selected for their known ability to enhance potency, and donor heterogeneity of MSC(AT). To do this, a matrix of assays consisting of morphometric analysis (of single cell suspensions), gene multiplex (58-gene panel), soluble factor analysis (10-analyte panel), in vitro monocyte/macrophage (denoted as Mo to represent a mixed population of both monocytes and macrophages) polarization (functional immunomodulatory assay), and in vitro human umbilical vein endothelial cell (HUVEC) tube formation assays (functional angiogenic assay) was applied. The matrix of MSC(AT) assays was sensitive to variations in CPPs (including 3D normoxic, 2D hypoxic, and 2D normoxic conditions), donor heterogeneity (N=5 human MSC(AT) donors), and to licensing with pro-inflammatory cytokines. Changes in the matrix of gene and protein expression profiles of MSC(AT), and morphological features correlated with functional immunomodulatory and angiogenic readouts by regression analyses to refine a panel of putative MSC(AT) CQAs. Desirability profiling of these putative CQAs allowed ranking of the effects of CPPs or donor heterogeneity on desired immunomodulatory or angiogenic properties. Schematic created using BioRender.com.

FIG. 2A-D shows curated gene expression panel was differentially sensitive to donor heterogeneity and select CPPs that enhance immunomodulatory and/or angiogenic potencies in an exemplary embodiment of the disclosure. FIG. 2A and FIG. 2B) Unbiased hierarchical clustering of normalized mRNA counts demonstrated clustering by CPPs (labelled dendrogram) and by donor (shaded regions in Donor chart indicate different donors) under both Licensed (FIG. 2A) and Unlicensed (FIG. 2B) conditions. FIG. 2C and FIG. 2D) Select genes were differentially expressed by modifying CPPs under Licensed (FIG. 2C) and Unlicensed (FIG. 2D) conditions. Multivariate linear regression, Benjamini-Yekutieli False Discovery Rate-corrected p values. *p<0.05, **p<0.01, ***p<0.001, ****p<0.0001 vs 2D-N. 3D-N: 3D Normoxic culture, 2D-N: 2D Normoxic culture, 2D-H: 2D Hypoxic culture. N=5 MSC(AT) donors, n=1 technical replicate due to high sensitivity of Nanostring measurements.

FIG. 3A-D shows curated anti-inflammatory and angiogenic soluble factors were differentially sensitive to donor heterogeneity and select CPPs that enhance immunomodulatory and/or angiogenic potencies in an exemplary embodiment of the disclosure. FIG. 3A and FIG. 3B) Unbiased hierarchical clustering of soluble factors demonstrated clustering by CPPs and donor under Licensed (FIG. 3A) and Unlicensed (FIG. 3B) conditions. FIG. 3C and FIG. 3D) Soluble factors with statistically significant differences between CPPs (left) or donors (right) are displayed for Licensed (FIG. 3C) and Unlicensed (FIG. 3D) conditions. One-way ANOVA, Tukey's post-hoc test. *p<0.05, **p<0.01, ***p<0.001, ****p<0.0001. Donors sharing same letter are significantly different (p<0.05). Horizontal bars: group mean, error bars: standard deviation. Data points represent mean of technical replicates for each donor and condition. 3D-N: 3D Normoxic culture, 2D-N: 2D Normoxic culture, 2D-H: 2D Hypoxic culture. N=5 MSC(AT) donors, n=2 technical replicates.

FIG. 4A-E shows in vitro MSC(AT)-mediated functional polarization of Mo was differentially sensitive to donor heterogeneity and select CPPs in an exemplary embodiment of the disclosure. FIG. 4A) Schematic of indirect co-culture experiment. MSC(AT) (2D-N, 2D-H, or 3D-N culture conditions) were co-cultured with human peripheral blood-derived monocytes/macrophages (Mϕ) for 24 h prior to removal of MSC(AT) and addition of lipopolysaccharide (LPS). FIG. 4B) TNFα secretion, a surrogate marker for pro-inflammatory Mϕs, showed significant reduction when co-cultured with 3D-N or 2D-H MSC(AT) compared to solo Mϕs. One-way ANOVA, Tukey post-hoc test. *p<0.05. FIG. 4C) Principal component (PC) analysis of median delta-delta Ct values of Mϕ genes, indicative of pro-inflammatory (CCR7, CD86, HLADRA, IL12A, IL1B, TREM1) or pro-resolving (CD163, CD206, HMOX1, IL10, CD274, STAB1) status, and normalized TNFα levels from each donor and culture condition (left). The corresponding loading plot of eigenvectors for each marker (right) indicates the relative contribution of each factor to the PCs. FIG. 4D) Principal component scores for PC1 and PC2 according to culture condition. PC1 scores were highest in the 3D-N co-culture condition. One-way ANOVA, Tukey post-hoc test. *p<0.05, **p<0.01, ***p<0.001, ****p<0.0001 relative to Mϕ SOLO condition or to groups indicated by brackets. FIG. 4E) Principal component scores for PC1 and PC2 according to MSC(AT) donor heterogeneity. Statistically significant differences between groups were observed in PC2 scores and are indicated by groups sharing the same letter. One-way ANOVA, Tukey post-hoc test, p<0.01. N=5 MSC(AT) donors, n=3 technical replicates/condition. Horizontal bars: group mean, error bars: standard deviation. 3D-N: 3D Normoxic culture, 2D-N: 2D Normoxic culture, 2D-H: 2D Hypoxic culture, n.s.: statistically non-significant. Schematic created using BioRender.com.

FIG. 5A-D shows in vitro MSC(AT) mediated functional angiogenic HUVEC tube formation assay was differentially sensitive to donor heterogeneity and CPPs in an exemplary embodiment of the disclosure. FIG. 5A) Schematic of experimental set-up for the tube formation assay. Conditioned medium (CM) was collected from MSC(AT) (2D-N, 2D-H, or 3D-N) after 24 h of incubation and added to HUVECs cultured on basement membrane extract for 6 h. FIG. 5B) Principal component (PC) analysis of fold-change values (relative to negative control) of twenty HUVEC tube formation image analysis readouts (left). The corresponding loading plot of eigenvectors (right) indicates the relative contribution of each factor to the PCs. FIG. 5C) Principal component scores for PC1 and PC2 according to culture condition. The positive control group (Pos; HUVECs cultured in pro-angiogenic medium) displayed significantly higher PC1 scores relative to the negative control (Neg; HUVECs cultured in basal medium), and to HUVECs cultured with conditioned medium derived from 2D-N or 3D-N MSC(AT) culture conditions. One-way ANOVA, Tukey post-hoc test. *p<0.05, **p<0.01 relative to positive control. FIG. 5D) Principal component scores for PC1 and PC2 according to MSC(AT) donor heterogeneity. Statistically significant differences between donors were observed for PC1 and PC2 scores and are indicated by donors sharing the same letter. One-way ANOVA, Tukey post-hoc test, p<0.05. N=5 MSC(AT) donors, n=2-3 technical replicates/condition. Horizontal bars: group mean, error bars: standard deviation. HUVEC: human umbilical vein endothelial cell, Pos: positive control, Neg: negative control, UCM: unconditioned medium, 3D-N: 3D Normoxic culture, 2D-N: 2D Normoxic culture, 2D-H: 2D Hypoxic culture, CM: conditioned medium, n.s.: statistically non-significant. Schematic created using BioRender.com.

FIG. 6A-E shows linear regression analysis revealed genes, soluble factors, and morphological features that correlate with anchor functional assays to identify a putative matrix of CQAs for MSC(AT) in an exemplary embodiment of the disclosure. FIG. 6A) Inverse correlation between PC1 composite scores from Mo polarization (as shown in FIG. 4A-FIG. 4E) and curated panels of MSC(AT) genes measured under Licensed conditions. THBS1; R²=0.5481, p=0.0025. CCN2; R²=0.3020, p=0.0418. FIG. 6B) Inverse correlation between PC1 composite scores from Mϕ polarization (as shown in FIG. 4A-FIG. 4E) and curated panels of MSC(AT) genes measured under Unlicensed conditions. CCN2; R²=0.4934, p=0.0035. TSG101; R²=0.4229, p=0.0087. THBS1; R²=0.3853, p=0.0135. PDGFA; R²=0.3754, p=0.0152. FIG. 6C) Single cell diameter of MSC(AT) inversely correlated to Mϕ polarization PC1 composite scores (R²=0.3515, p=0.0199). FIG. 6D) Positive correlation between HUVEC tube formation PC1 composite scores (as shown in FIG. 5A-FIG. 5D) and levels of VEGF protein in MSC(AT) conditioned medium under Licensed conditions (R²=0.3048, p=0.0328). FIG. 6E) Inverse correlation between HUVEC tube formation PC1 scores and curated panels of MSC(AT) genes measured under Licensed conditions. Only correlations with R²≥0.3 are shown. N=14-15. 3D-N: 3D Normoxic culture, 2D-N: 2D Normoxic culture, 2D-H: 2D Hypoxic culture.

FIG. 7A-B shows statistical rankings of matrix of putative CQAs ranked different CPP conditions known to enhance MSC potency, and donors for optimal immunomodulation and angiogenic functionalities in an exemplary embodiment of the disclosure. Desirability analysis was performed on putative CQAs selected for evaluation of immunomodulatory (FIG. 7A) and angiogenic (FIG. 7B) functionalities. FIG. 7A) Desirability profiling ranked 3D-N CPP conditions higher than 2D-H and 2D-N for overall immunomodulatory desirability scores, while scores were similar across donors. FIG. 7B) Desirability profiling ranked Donors 1, 2, and 3 higher than Donor 4 for overall angiogenic desirability scores, while scores were similar across various CPPs. One-way ANOVA, Tukey post-hoc test. *p<0.05. Donors sharing same letter indicate statistically significant differences (p<0.05). 3D-N: 3D Normoxic culture, 2D-N: 2D Normoxic culture, 2D-H: 2D Hypoxic culture.

FIG. 8A-C shows that MSC(AT) cultured using 3D-N and 2D-H conditions satisfied surface marker criteria by IFATS and ISCT in an exemplary embodiment of the disclosure. FIG. 8A shows MSC(AT) cultured under 2D-N, 2D-H and 3D-N conditions satisfied surface marker expression criteria and were CD90⁺CD73⁺CD44⁺CD13⁺CD34⁻CD31⁻CD45⁻. FIG. 8B shows CD105 was cleaved under enzymatic conditions required for dissociating 3D cell aggregates for flow cytometry analysis. FIG. 8C shows a western blot demonstrating expression of CD105 by MSC(AT) cultured using 2D-N, 2D-H, and 3D-N methods. N=2 donors. D: Donor, 3D-N: 3D Normoxic culture, 2D-N: 2D Normoxic culture, 2D-H: 2D Hypoxic culture.

FIG. 9A-C shows morphometric characterization of MSC(AT) cultured under various CPP conditions known to enhance immunomodulatory and/or angiogenic MSC potency in an exemplary embodiment of the disclosure. FIG. 9A) Representative photomicrographs of MSC(AT) cultured using each CPP. Scale bars: 500 μm. FIG. 9B and FIG. 9C) Dissociated 3D aggregates showed reduced single cell diameter (FIG. 9B) and greater circularity (FIG. 9C) relative to 2D-N and 2D-H conditions. One-way ANOVA, Tukey post-hoc test. *p<0.05, **p<0.01, ***p<0.001, ****p<0.0001. N=5 MSC(AT) donors, n=3 technical replicates. Horizontal bars: group mean, error bars: standard deviation. 3D-N: 3D Normoxic culture, 2D-N: 2D Normoxic culture, 2D-H: 2D Hypoxic culture.

FIG. 10 shows gene expression analysis of licensed MSC(AT) demonstrated no advantage of combining 3D and hypoxic culture conditions in an exemplary embodiment of the disclosure. Two MSC(AT) donors (D1—top, and D2—bottom) were cultured overnight (16-20 h) using 2D or 3D conditions with normoxic or hypoxic culture, followed by an additional 24 h of 2D or 3D culture in normoxic or hypoxic conditions with addition of pro-inflammatory licensing factors prior to harvesting samples for gene expression analysis by qPCR. 2D/3D indicates culture under 2D or 3D conditions. First “N” or “H” denotes culture under normoxic (N) or hypoxic (H) conditions during the initial 2D/3D culture period. Second “N” or “H” denotes culture under normoxic (N) or hypoxic (H) conditions during the licensing periods. Two-way ANOVA, Tukey post-hoc test. N.s.: statistically non-significant comparisons between 3D groups indicated. Horizontal bars: group median. N=2 MSC(AT) donors, n=3 technical replicates.

FIG. 11 shows gene expression analysis of licensed MSC(AT) demonstrated no effect of IL-6 treatment on MSC(AT) cultured under 2D-N conditions in an exemplary embodiment of the disclosure. Two MSC(AT) donors (D1—top, and D2—bottom) cultured under 2D-N conditions with addition of IL-6 (at same concentration and duration as used for 3D-N culture) showed no significant changes in a panel of genes that were differentially expressed by MSC(AT) cultured under 3D-N conditions. Two-way ANOVA, Tukey post-hoc test. N.s.: statistically non-significant. 2D-N Control: 2D Normoxic culture without IL-6 treatment. Horizontal bars: group median. N=2 MSC(AT) donors, n=3 technical replicates.

FIG. 12A-C shows MSC(AT)-mediated MO polarization in an exemplary embodiment of the disclosure. FIG. 12A) Box-and-whisker plots display changes in gene expression relative to Mϕ cultured alone. Two-way ANOVA, Tukey post-hoc test. *p<0.05, **p<0.01, ****p<0.0001 vs Mϕ cultured alone. 3D-N: 3D Normoxic culture, 2D-N: 2D Normoxic culture, 2D-H: 2D Hypoxic culture. N=5 MSC(AT) donors, n=3 technical replicates. FIG. 12B and FIG. 12C) Factor loading plot shows the relative contribution of each factor to principal component 1 (PC1) (FIG. 12B) and PC2 (FIG. 12C) corresponding to the PC analysis in FIG. 4C. Factors are listed in order of decreasing loadings. Loadings close to +1 have a greater relative contribution to the respective PC.

FIG. 13A-C shows HUVEC tube formation analysis in an exemplary embodiment of the disclosure. FIG. 13A) Phase-contrast images of HUVEC tube formation assay. Scale: 500 μm. POS: positive control, NEG: negative control, UCM: unconditioned medium, 3D-N: 3D Normoxic culture, 2D-N: 2D Normoxic culture, 2D-H: 2D Hypoxic culture, CM: conditioned medium. FIG. 13B and FIG. 13C) Factor loading plot shows the relative contribution of each factor to principal component 1 (PC1) (FIG. 13B) and PC2 (FIG. 13C) corresponding to the PC analysis in FIG. 5B. Factors are listed in order of decreasing loadings. Loadings close to +1 have a greater relative contribution to the respective PC.

FIG. 14 shows principal component analysis of HUVEC tube formation image analysis read-outs showed differential effects of experimental batch, MSC(AT) donor heterogeneity, and variations in CPP conditions in an exemplary embodiment of the disclosure. Principal component (PC) analysis of fold-change values (relative to negative control) of twenty HUVEC tube formation image analysis read-outs, including all biological and technical replicates (left) with corresponding loading plot of eigenvectors for each measurement (right). PC1 scores corresponding to technical replicates for each culture condition and donor within experimental batch 1 are shown in Table 12 and batch 2 are shown in Table 13. N=5 MSC(AT) donors, n=2-3 technical replicates/condition. Horizontal bars: group mean, error bars: standard deviation. Pos: positive control, Neg: negative control, UCM: unconditioned medium, 3D-N: 3D Normoxic culture, 2D-N: 2D Normoxic culture, 2D-H: 2D Hypoxic culture, CM: conditioned medium. Note, this data includes biological and technical replicates while FIG. 5A-5D is based on median values of biological replicates only.

FIG. 15A-D shows previously identified CQA genes distinguish donor MSC(M) basal immunomodulatory fitness for responders versus non-responders in an exemplary embodiment of the disclosure. FIG. 15A) Principal component (PC) analysis of MSC(M) expression of putative CQA genes expressed under licensed conditions. The corresponding loading plot of eigenvectors (FIG. 15B) indicates the relative contribution of each gene to the PC1 and PC2 axes. FIG. 15C) MSC(M) derived from Function-Pain Responders, Function Responders, and Pain Responders displayed significantly lower PC1 scores relative to non-responders. Student's t-test, ***p<0.001, ****p<0.0001. Horizontal lines: group mean; error bars: standard deviation. FIG. 15D) Significant negative correlations between PC1 scores and the percentage change at 24-months relative to baseline for KOOS ADL (R_{S}=−0.7167, p=0.0298), pain (R_{S}=−0.700, p=0.0358), symptoms (R_{S}=−0.7500, p=0.0199), and overall KOOS (R_{S}=−0.7000, p=0.0358). Percent change in KOOS values were made relative to baseline and calculated such that positive values represent improvement. Spearman's correlation. N=9 MSC(M) donors, n=2-3 replicates/donor. ADL: function in daily living; Pt.: patient ID; MO: month; KOOS: knee injury and osteoarthritis outcome score. N=9 MSC(M) donors; n=2-3 replicates/donor.

FIG. 16A-D shows previously identified CQAs distinguish donor MSC(M) immunomodulatory fitness for responders versus non-responders in an exemplary embodiment of the disclosure. FIG. 16A) Schematic of in vitro monocyte/macrophage (Mϕ) polarization experiment. Indirect co-cultures of MSC(M) with human peripheral blood-derived was performed for 48 h prior to removal of MSC(M) and addition of lipopolysaccharide (LPS) for 4 h incubation. FIG. 16B-D) Summary of Mϕ gene expression after co-culture with Function-Pain Responders (FIG. 16B), Function Responders (FIG. 16C), and Pain Responders (FIG. 16D). Box-and-whisker plots (left) display changes in gene expression relative to Mϕ cultured alone (Solo; dotted line). Horizontal line: median; hinges: first and third quartiles; whiskers: range. Two-way ANOVA, Tukey's post-hoc test. *p<0.05, **p<0.01, ***p<0.001, ****p<0.0001 relative to Solo condition or to groups indicated by brackets. Discriminant canonical plots (right) of −ddCt values relative to Solo condition. Inner ellipses indicate 95% confidence for the mean of each group; outer ellipses indicate the normal region estimated to contain 50% of the population for each group. N=10 MSC(M) donors, n=2-3 replicates/donor. ADL: function in daily living; Pt.: patient ID; MO: month; KOOS: knee injury and osteoarthritis outcome score.

FIG. 17A-D shows desirability analysis of MSC(M) CQA genes based on previous correlations of MSC(AT) genes with in vitro monocyte/macrophage polarization (training dataset, Robb et al., FII 2022) partially distinguishes basal immunomodulatory fitness of MSC(M) derived from responders versus non-responders in an exemplary embodiment of the disclosure. FIG. 17A) Overall desirability scores calculated based on equal weighting of all genes were significantly increased in Function Responders, but not Function-Pain Responders or Pain Responders. Student's unpaired t-test. **p<0.01. FIG. 17B) Overall desirability scores calculated based on equal weighting of all genes were positively correlated with changes in Overall KOOS scores, including percent change (r_{S}=0.6333, p=0.0671) and delta values (r_{S}=0.6611, p=0.0525), at 24 months relative to baseline. Spearman's correlation. FIG. 17C) Overall desirability scores calculated based on individual gene weightings were significantly increased in Function Responders but not Function-Pain Responders or Pain Responders. Student's unpaired t-test. *p<0.05, **p<0.01, ***p<0.001. FIG. 17D) Overall desirability scores calculated based on individual gene weightings were positively correlated with changes in Overall KOOS scores, including percent change (r_{S}=0.5833, p=0.0992) and delta values (r_{S}=0.6025, p=0.0860), at 24 months relative to baseline. Spearman's correlation. N=9 MSC(M) donors, n=2-3 replicates/donor. MO: month; KOOS: knee injury and osteoarthritis outcome score.

FIG. 18A-B shows correlation of MSC(M) desirability scores (test dataset) calculated based on previous analysis of MSC(AT) with changes in KOOS at 12 months relative to baseline (training dataset) in an exemplary embodiment of the disclosure. FIG. 18A) Overall desirability scores calculated based on equal weighting of all genes were positively correlated with changes in Overall KOOS scores, including percent change (r_{S}=0.5500, p=0.1250) and delta values (r_{S}=0.6833, p=0.0424), at 12 months relative to baseline. FIG. 18B) Overall desirability scores calculated based on individual gene weightings were positively correlated with changes in Overall KOOS scores, including percent change (r_{S}=0.5167, p=0.1544) and delta values (r_{S}=0.6167, p=0.0769), at 12 months relative to baseline. Spearman's correlation. N=9 MSC(M) donors, n=2-3 replicates/donor. MO: month; KOOS: knee injury and osteoarthritis outcome score.

FIG. 19A-D shows refined desirability analysis based on correlations of MSC(M) CQA genes with 24-month KOOS (test dataset) shows improved ability to distinguish immunomodulatory fitness for responders versus non-responders in an exemplary embodiment of the disclosure. FIG. 19A) Overall desirability scores calculated based on equal weighting of all genes were significantly increased in Function-Pain Responders, Function Responders, and Pain Responders. Student's unpaired t-test. *p<0.05, **p<0.01, ***p<0.001. FIG. 19B) Overall desirability scores calculated based on equal weighting of all genes were significantly positively correlated with changes in Overall KOOS scores, including percent change (r_{S}=0.6667, p=0.0499) and delta values (r_{S}=0.6862, p=0.0412), at 24 months relative to baseline. Spearman's correlation. FIG. 19C) Overall desirability scores calculated based on individual gene weightings were significantly increased in Function-Pain Responders, Function Responders, and Pain Responders. Student's unpaired t-test. *p<0.05, **p<0.01, ***p<0.001. FIG. 19D) Overall desirability scores calculated based on equal weighting of all genes were significantly positively correlated with changes in Overall KOOS scores, including percent change (r_{S}=0.6667, p=0.0499) and delta values (r_{S}=0.6667, p=0.0412), at 24 months relative to baseline. Spearman's correlation. N=9 MSC(M) donors, n=2-3 replicates/donor. MO: month; KOOS: knee injury and osteoarthritis outcome score.

FIG. 20A-D shows refined desirability analysis based on correlations of MSC(M) genes with 12-month KOOS distinguishes immunomodulatory fitness for responders versus non-responders in an exemplary embodiment of the disclosure. FIG. 20A) Overall desirability scores calculated based on equal weighting of all genes were significantly increased in Function-Pain Responders, Function Responders, and Pain Responders. Student's unpaired t-test. ***p<0.001, ****p<0.0001. FIG. 20B) Overall desirability scores calculated based on equal weighting of all genes were significantly positively correlated with changes in Overall KOOS scores, including percent change (r_{S}=0.6000, p=0.0876) and delta values (r_{S}=0.8000, p=0.0096), at 12 months relative to baseline. Spearman's correlation. FIG. 20C) Overall desirability scores calculated based on individual gene weightings were significantly increased in Function-Pain Responders, Function Responders, and Pain Responders. Student's unpaired t-test. **p<0.01, ***p<0.001. FIG. 20D) Overall desirability scores calculated based on individual gene weightings were significantly positively correlated with changes in Overall KOOS scores, including percent change (r_{S}=0.7333, p=0.0246) and delta values (r_{S}=0.8500, p=0.0037), at 12 months relative to baseline. Spearman's correlation. N=9 MSC(M) donors, n=2-3 replicates/donor. MO: month; KOOS: knee injury and osteoarthritis outcome score.

### DESCRIPTION OF VARIOUS EMBODIMENTS

The following is a detailed description provided to aid those skilled in the art in practicing the present disclosure. Unless otherwise defined, all technical and scientific terms used herein have the same meaning as commonly understood by one of ordinary skill in the art to which this disclosure belongs. The terminology used in the description herein is for describing particular embodiments only and is not intended to be limiting of the disclosure. All publications, patent applications, patents, figures and other references mentioned herein are expressly incorporated by reference in their entirety.

Further, the definitions and embodiments described in particular sections are intended to be applicable to other embodiments herein described for which they are suitable as would be understood by a person skilled in the art. For example, in the following passages, different aspects of the disclosure are defined in more detail. Each aspect so defined may be combined with any other aspect or aspects unless clearly indicated to the contrary. In particular, any feature described herein may be combined with any other feature or features described herein.

### I. Definitions

As used herein, the following terms may have meanings ascribed to them below, unless specified otherwise. However, it should be understood that other meanings that are known or understood by those having ordinary skill in the art are also possible, and within the scope of the present disclosure. All publications, patent applications, patents, and other references mentioned herein are incorporated by reference in their entirety. In the case of conflict, the present specification, including definitions, will control. In addition, the materials, methods, and examples are illustrative only and not intended to be limiting.

Where a range of values is provided, it is understood that each intervening value, to the tenth of the unit of the lower limit unless the context clearly dictates otherwise, between the upper and lower limit of that range and any other stated or intervening value in that stated range is encompassed within the description. Ranges from any lower limit to any upper limit are contemplated. The upper and lower limits of these smaller ranges which may independently be included in the smaller ranges is also encompassed within the description, subject to any specifically excluded limit in the stated range. Where the stated range includes one or both of the limits, ranges excluding either or both of those included limits are also included in the description.

All numerical values herein are modified by “about” or “approximately” the indicated value, and take into account experimental error and variations that would be expected by a person having ordinary skill in the art.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies or unless the context suggests otherwise to a person skilled in the art.

As used herein, the singular forms “a”, “an”, and “the” include plural references unless the context clearly dictates otherwise.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of” or, when used in the claims, “consisting of” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, all transitional phrases such as “comprising,” “including,” “carrying,” “having,” “containing,” “involving,” “holding,” “composed of,” and the like are to be understood to be inclusive or be open-ended, i.e., to mean including but not limited to, and do not exclude additional, unrecited elements or process steps. Only the transitional phrases “consisting of” and “consisting essentially of” shall be closed or semi-closed transitional phrases, respectively.

The term “consisting” and its derivatives as used herein are intended to be closed terms that specify the presence of the stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

As used herein, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, in certain methods described herein that include more than one step or act, the order of the steps or acts of the method is not necessarily limited to the order in which the steps or acts of the method are recited unless the context indicates otherwise.

Further, the definitions and embodiments described in particular sections are intended to be applicable to other embodiments herein described for which they are suitable as would be understood by a person skilled in the art. For example, in the following passages, different aspects of the disclosure are defined in more detail. Each aspect so defined may be combined with any other aspect or aspects unless clearly indicated to the contrary. In particular, any feature described herein may be combined with any other feature or features described herein.

### II. Methods

The present inventors have identified critical quality attributes relating to particular mRNA levels, protein levels and morphometric features that are indicative of mesenchymal stromal cells immunomodulatory fitness or angiogenic fitness.

Accordingly, in one aspect, the present disclosure provides a method of determining immunomodulatory fitness of a sample of mesenchymal stromal cells (MSC) comprising:
determining a sample critical quality attribute (CQA) profile, the sample CQA profile comprising:
a) from the sample harvested:
   a. in the presence of proinflammatory cytokines stimulation post-harvest: the mRNA levels of at least one, at least two, at least three, at least four, at least five, at least six, at least seven or all of the following genes: THBS1, CCN2, EDN1, ACTA2, PDCD1LG2, TNFAIP6, ANGPT1, CXCL8; and/or
   b. in the absence of the proinflammatory cytokines stimulation post-harvest: the mRNA levels of at least one, at least two, at least three, at least four, at least five, at least six, at least seven, at least eight or all of the following genes: CCN2, TSG101, THBS1, PDGFA, VEGFA, EDIL3, ACTA2, ANGPT1, ANG and/or the level of TGF-beta protein; and/or
b) from a single cell suspension of the sample post-harvest without pro-inflammatory cytokines stimulation: cell diameter and/or cell circularity;
determining the level of similarity of said sample CQA profile to one or more control profiles, wherein (i) a high level of similarity of the sample CQA profile to an immunomodulatory specific control profile; (ii) a low level of similarity to a non-immunomodulatory specific control profile; and/or (iii) a higher level of similarity to an immunomodulatory specific control profile than to a non-immunomodulatory specific control profile indicates the cells have basal immunomodulatory fitness.

In an embodiment, a higher level of similarity to the immunomodulatory specific control profile than to the non-immunomodulatory specific control profile is indicated by a higher correlation value computed between the sample profile and the immunomodulatory specific control profile than an equivalent correlation value computed between the sample profile and the non-immunomodulatory specific control profile, optionally wherein the correlation value is a correlation coefficient.

In another aspect, there is provided a method of determining angiogenic fitness of derived mesenchymal stromal cells (MSC) comprising:
determining a sample critical quality attribute (CQA) profile, the sample CQA profile comprising:
a) from the sample harvested and cultured:
   a. in the presence of proinflammatory cytokines stimulation post-harvest: the mRNA levels of at least one, at least two, at least three, at least four, at least five or all the following genes: SMAD7, HGF, ANG, IDO1, TSG101, CXCL8 and/or the level of VEGF protein; and/or
   b. in the absence of the proinflammatory cytokines stimulation post-harvest: the mRNA levels of the following genes: SOX9 and/or TNFAIP6 and/or the level of VEGF protein;
determining the level of similarity of said sample CQA profile to one or more control profiles, wherein (i) a high level of similarity of the sample CQA profile to an angiogenic specific control profile; (ii) a low level of similarity to a non-angiogenic specific control profile; and/or (iii) a higher level of similarity to an angiogenic specific control profile than to a non-angiogenic specific control profile indicates the cells have basal angiogenic fitness.

In an embodiment, a higher level of similarity to the angiogenic specific control profile than to the non-angiogenic specific control profile is indicated by a higher correlation value computed between the sample profile and the angiogenic specific control profile than an equivalent correlation value computed between the sample profile and the non-angiogenic control profile, optionally wherein the correlation value is a correlation coefficient.

THBS1 can be from any organism or source, and optionally as shown in NM_003246.2. CCN2 can be from any organism or source, and optionally as shown in NM_001901.2. EDN1 can be from any organism or source, and optionally as shown in NM_001168319.1. ACTA2 can be from any organism or source, and optionally as shown in NM 001613.1. PDCD1LG2 can be from any organism or source, and optionally as shown in NM 025239.3. TNFAIP6 can be from any organism or source, and optionally as shown in NM_007115.2. ANGPT1 can be from any organism or source, and optionally as shown in NM_001146.3. CXCL8 can be from any organism or source, and optionally as shown in NM_000584.2. TSG101 can be from any organism or source, and optionally as shown in NM 006292.2. PDGFA can be from any organism or source, and optionally as shown in NM_002607.5. VEGFA can be from any organism or source, and optionally as shown in NM 001025366.1. EDIL3 can be from any organism or source, and optionally as shown in NM_005711.3. ANG can be from any organism or source, and optionally as shown in NM 001145.4. SMAD7 can be from any organism or source, and optionally as shown in NM_005904.2. HGF can be from any organism or source, and optionally as shown in NM_000601.4. IDO1 can be from any organism or source, and optionally as shown in NM_002164.5. SOX9 can be from any organism or source, and optionally as shown in NM_000346.2. With respect to the VEGF protein, it can be from any organism or source, and optionally as shown in NP_001020537, NP_001020538, NP_001020539, NP_001020540 or NP_001020541. With respect to the TGF-beta protein, it can be from any organism or source, and optionally as shown in NP_000651, NP_001129071, NP_003229, NP_001316867, NP_001316868, and NP_003230.

MSCs can be obtained from a variety of tissue sources, including without limitation, adipose tissue, bone marrow, dental pulp, synovium, placenta, and umbilical cord, for example umbilical cord blood and umbilical cord tissue (i.e., Wharton's jelly). Accordingly, in one embodiment, the MSCs are derived from adipose tissue, bone marrow, dental pulp, synovium, placenta, or umbilical cord. In another embodiment, the MSCs are derived from adipose tissue. In yet another embodiment, the MSCs are derived from bone marrow.

MSCs can be derived from any animal, including dogs, cats and humans. In an embodiment, the MSCs are for veterinary use and are derived from dogs or cats. In another embodiment, the MSCs are for medical use and are derived from humans.

In an embodiment, the proinflammatory cytokines stimulation post-harvest comprises IFNγ, TNFα and/or IL-1β. In an embodiment, the proinflammatory cytokines comprise 30 ng/ml of IFNγ, 10 ng/ml of TNFα and/or 5 ng/ml of IL-1β. In another embodiment, the proinflammatory cytokines stimulation post-harvest comprises IFNγ, TNFα and IL-1β.

The term CQA as used herein refers to the critical quality attributes, such as the specified mRNA/transcript levels, protein, and morphometric features that are indicative of either immunomodulatory fitness or angiogenic fitness of MSCs.

mRNA levels of each gene can be obtained by measuring mRNA expression, for example, by qPCR, or by directly quantifying mRNA, for example, by RNA-Sequencing or Nanostring. In an embodiment, mRNA levels are determined by measuring mRNA count.

Protein levels can be determined by measuring the amount of protein in the conditioned media or by measuring intracellular levels from the sample. In an embodiment, protein levels are determined by measuring the amount of the particular protein in the conditioned media.

Cell diameter can be determined by image analysis performed by an automated cell counter or using other image analysis software, or through use of flow cytometry techniques. In an embodiment, cell diameter is measured using an automated cell counter.

Cell circularity can be determined by image analysis performed by an automated cell counter or using other image analysis software. In an embodiment, cell circularity is measured using an automated cell counter.

The term “patient-reported outcome measure score” or “PROM score” as used herein refers to any patient-reported symptomatic variable associated with a clinical phenotype of osteoarthritis. A PROM score can, for example, be derived from an assessment of osteoarthritis and/or a physical activity assessment. The PROM score may, for example, be an absolute, relative, interval, or ordinal value.

The term “basal immunomodulatory fitness” as used herein refers to MSCs with an ability to modulate immune responses to a sufficient degree so as to be measurable.

The term “basal angiogenic fitness” as used herein refers to MSCs with an ability to modulate angiogenesis to a sufficient degree so as to be measurable.

As used herein, the term “sample critical quality attribute profile” or “sample CQA profile” or “sample profile” refers to the levels of mRNA of the identified genes, the levels of the identified protein(s), as well as the indicated morphometric measurements of a particular sample or data set.

In some embodiments, the sample CQA profile is compared to one or more control profiles from a donor that has been grown under the same processing conditions, called critical processing parameters or CPPs.

The term “CPPs” or “critical processing parameters” as used herein refers to the conditions in which the MSCs are grown that may influence the fitness of the sample and include, without limitation, cells grown in 3D normoxic (3D-N), 2D hypoxic (2D-H), or 2D normoxic (2D-N) conditions. In one embodiment, the MSCs are grown in 3D-N conditions supplemented with IL-6 to enhance MSC fitness. In an embodiment, the concentration of IL-6 is from 0.1 to 10 ng/ml, optionally about 2 ng/ml. For example, 3D-N culture may be performed by harvesting MSC(AT) from adherent flasks and plating cells on ultra-low attachment surfaces (Corning, Corning, USA) at 26,700 cells/cm² and 200,000 cells/mL in medium supplemented with 2 ng/ml IL-6.

The term “donor” as used herein refers to an individual from which tissue was obtained to derive MSCs.

The control profile may be a reference value and/or may be derived from one or more samples, optionally from historical CQA data from a pool of samples from the same donor under the same CPPs. The control profile may be a reference value and/or may be derived from one or more samples, optionally from historical CQA data from a pool of samples from the same donor under the same processing conditions. In an embodiment, the control profile is a value that is continually updated as further samples are collected and immunomodulatory or angiogenic fitness is measured and correlated. It will be understood that the control profile represents an average of the levels for the selected genes, proteins and features described herein. Average values may, for example, be the mean values or median values.

For example, an “immunomodulatory specific control profile” may be generated by measuring the mRNA levels of the specified genes, the protein levels of the specified proteins and/or the specified morphometric features for those samples known to have basal immunomodulatory fitness. Similarly, a “non-immunomodulatory control profile” may be generated by measuring the mRNA levels of the specified genes, the protein levels of the specified proteins and/or the specified morphometric features for those samples known not to have basal immunomodulatory fitness.

Further, an “angiogenic specific control profile” may be generated by measuring the mRNA levels of the specified genes, the protein levels of the specified proteins and/or the specified morphometric features for those samples known to have basal angiogenic fitness. Similarly, a “non-angiogenic control profile” may be generated by measuring the mRNA levels of the specified genes, the protein levels of the specified proteins and/or the specified morphometric features for those samples known not to have basal angiogenic fitness.

Methods of determining the similarity between profiles are well known in the art. Methods of determining similarity may in some embodiments provide a non-quantitative measure of similarity, for example, using visual clustering. In other embodiments, similarity may be determined using methods which provide a quantitative measure of similarity.

In an embodiment, similarity may be measured by computing a “correlation coefficient”, which is a measure of the interdependence of random variables that ranges in value from −1 to +1, indicating perfect negative correlation at −1, absence of correlation at zero, and perfect positive correlation at +1. In an embodiment, the correlation coefficient may be a linear correlation coefficient, for example, a Pearson product-moment correlation coefficient.

A Pearson correlation coefficient (r) is calculated using the following formula:

$$r = \frac{{\sum }_{i} ( {x}_{i} - \overline{x} ) ( {y}_{i} - \overline{y} )}{\sqrt{{\sum }_{i} {( {x}_{i} - \overline{x} )}^{2}} \sqrt{{\sum }_{i} {( {y}_{i} - \overline{y} )}^{2}}}$$

In an embodiment, the correlation coefficient may be a monotonic correlation coefficient, for example, a Spearman's rank correlation coefficient.

A Spearman's rank correlation coefficient (p) is calculated using the following formula:

$${r}_{S} = {ρ}_{R ( X ) , R ( Y )} = \frac{cov ( R ( X ) , R ( Y ) )}{{σ}_{R ( X )} {σ}_{R ( Y )}}$$

where cov(R(X), R(Y) is the covariance of rank variables and OR(x) and OR(Y) are their standard deviations.

In one embodiment, x and y are the expression values of the mRNA, protein or the morphometric measurements in a sample profile and a control profile, respectively. In another embodiment, X and Y are the PROM scores or the immunomodulatory fitness score in a sample profile and a control profile, respectively.

In an embodiment, a correlation coefficient calculated between a sample CQA profile and a control profile indicates a high level of similarity to the control profile when the correlation coefficient has an absolute value between 0.5 to 1, optionally between 0.75 to 1, and a low level of similarity to the control profile when the correlation coefficient has an absolute value between 0 to 0.5, optionally between 0 to 0.25.

It will be appreciated that any “correlation value” which provides a quantitative scaling measure of similarity between profiles may be used to measure similarity.

A sample CQA profile may be identified as having basal immunomodulatory fitness, where the sample profile has high similarity to the immunomodulatory specific control profile, low similarity to the non-immunomodulatory specific control profile, or higher similarity to the immunomodulatory specific control profile than to the non-immunomodulatory specific control profile. Conversely, a sample profile may be identified as non-immunomodulatory, where the sample CQA profile has high similarity to the non-immunomodulatory specific control profile, low similarity to the immunomodulatory specific control profile, or higher similarity to the non-immunomodulatory specific control profile than to the immunomodulatory specific control profile.

A sample CQA profile may be identified as having basal angiogenic fitness, where the sample profile has high similarity to the angiogenic specific control profile, low similarity to the non-angiogenic specific control profile, or higher similarity to the angiogenic specific control profile than to the non-angiogenic specific control profile. Conversely, a sample profile may be identified non-angiogenic, where the sample CQA profile has high similarity to the non-angiogenic specific control profile, low similarity to the angiogenic specific control profile, or higher similarity to the non-angiogenic specific control profile than to the angiogenic specific control profile.

For example, in an embodiment, a sample profile may be identified as having immunomodulatory fitness based on calculation of a score, which generally is defined by the following formula:

score(B)=r(B,immunomodulatory or angiogenic profile)−r(B,control profile)

where r is the Pearson correlation coefficient, and B is a vector of mRNA/protein levels and morphometric measurements across the selected CQA.

A sample profile with a positive immunomodulatory fitness score is more similar to the immunomodulatory specific control profile across the selected CQA, and is therefore classified as “having basal immunomodulatory fitness;” whereas a sample with a negative immunomodulatory fitness score is more similar to the non-immunomodulatory specific control profile across the selected CQA, and is classified as “not having basal immunomodulatory fitness”. Similarly, a sample profile with a positive angiogenic fitness score is more similar to the angiogenic specific control profile across the selected CQA, and is therefore classified as “having basal angiogenic fitness”; whereas a sample with a negative angiogenic fitness score is more similar to the non-immunomodulatory specific control profile across the selected CQA, and is classified as “not having basal angiogenic fitness”.

In an embodiment, the CQA profiles for either immunomodulatory fitness or angiogenic fitness are analyzed using combinatorial or multivariate dimension reduction statistical or mathematical methods. Combinatorial or multivariate dimension reduction statistical or mathematical methods may be clustering analysis or desirability analyses to enable comparisons between multivariate CQA profiles with dimension-reduced empirical values.

In an embodiment, the clustering analysis is principal component analysis. Principal component analysis is applied as unbiased tool to visualize similarity between the multivariate CQA profiles of different groups (e.g. a non-immunomodulatory specific control profile, a non-angiogenic specific control profile, and a sample profile). In another embodiment, the clustering analysis is canonical correlation analysis. Canonical correlation analysis is applied as a supervised technique to test whether multivariate CQA profiles of different groups are statistically distinct, and to provide information on their level of similarity.

Desirability analysis or desirability profiling is based on methods developed by Derringer and Suich, 1980 [47], incorporated herein by reference. In an embodiment, desirability analysis is applied to rank the immunomodulatory or angiogenic fitness of a sample based on the combination of CQAs measured to facilitate relative comparisons between sample or control profiles.

The present inventors have shown that quantitative values can be determined for the CQAs for determining immunomodulatory fitness or angiogenic fitness. A set of CQAs can be used to assess samples from different donors or samples grown using different critical processing parameters (CPPs) in order to identify donors or CPPs that give rise to MSCs with basal immunomodulatory or angiogenic fitness. This evaluation can be considered a training dataset for particular donors or CPPs. Once values are obtained for a particular donor or CPPs, such values can then be used to assess fitness of samples from the same donor or grown under the same CPPs, for example, as a pass or fail for manufacturing processes.

Accordingly, also provided herein is a method of determining quantitative values for a set of CQAs for assessing immunomodulatory fitness of samples of mesenchymal stromal cells (MSC), wherein the MSC samples are from donors and/or from samples grown using different critical processing parameters (CPPs), the method comprising:
a) determining the values of the set of CQAs for each sample; wherein the set of CQAs comprise:
   a. from the sample cultured and harvested:
      i. in the presence of proinflammatory cytokines stimulation post-harvest: the mRNA levels of at least one, at least two, at least three, at least four, at least five, at least six, at least seven or all of the following genes: THBS1, CCN2, EDN1, ACTA2, PDCD1LG2, TNFAIP6, ANGPT1, CXCL8; and/or
      ii. in the absence of the proinflammatory cytokines stimulation post-harvest: the mRNA levels of at least one, at least two, at least three, at least four, at least five, at least six, at least seven, at least eight or all of the following genes: CCN2, TSG101, THBS1, PDGFA, VEGFA, EDIL3, ACTA2, ANGPT1, ANG and/or the level of TGF-beta protein; and/or
   b. from a single cell suspension of the sample: cell diameter and/or cell circularity post-harvest without pro-inflammatory cytokine stimulation;
b) testing each sample of MSC for immunomodulatory fitness using in vitro assays or obtaining immunomodulatory fitness status of the sample;
c) conducting a regression analysis between each CQA of a) with the immunomodulatory fitness of b); determining minimum and maximum data values of each CQA of the samples and using multivariate dimension reduction statistical or mathematical methods to assign minimization or maximization functions to indicate whether higher or lower values are desirable based on the directionality of correlation respectively.

Desirability analysis may be applied to analyze the profile of putative CQAs and to assign empirical rankings for donors and CPP conditions that result in desirable MSC immunomodulatory fitness.

Accordingly, in an embodiment, the method further comprises
d) assigning a weighting to each CQA for desirability analysis based on the R² values from the regression analysis; and
e) use of desirability analysis methods to assign a score from zero to one to each sample based on the range of data values; wherein zero is undesirable and one is highly desirable to obtain a set of values for MSCs for each donor grown using particular CPPs.

The testing for immunomodulatory fitness in step b) may be accomplished by measuring MSC-mediated polarization of monocyte/macrophages toward inflammation-resolving phenotypes within in vitro co-cultures. Other in vitro immunomodulatory assays are known in the art and may also be used, including, without limitation, assays including MSCs or their secretome or components of their secretome in cultures with regulatory T cell (Treg) induction, suppression of peripheral blood mononuclear cells (PBMC) or T cell proliferation.

Alternatively, the testing for immunomodulatory fitness in step b) may comprise evaluating immunomodulatory effects of MSC using preclinical animal models. Such animal models, include, without limitation, acute inflammatory models such as the zymosan-induced peritonitis mouse model, fibrotic models, and autoimmune disease models and chronic inflammatory models. In these models, MSCs are administered and the immunomodulatory effects of the cells can be measured by histological techniques, biomarker analysis, or other efficacy readouts specific to the model.

Testing the MSCs for immunomodulatory fitness is not required where the immunological fitness is already known. Accordingly, in an embodiment, the immunological fitness status of the sample in step b) is obtained from a clinical sample for which the immunomodulatory fitness or efficacy of the MSCs were previously evaluated clinically. For example, where the clinical sample is from a clinical study or pre-clinical or other correlated read-outs.

Alternatively, obtaining the immunomodulatory fitness status of the sample in step b) can be determined clinically, for example by treating a subject having an MSC-treatable disease with the sample and evaluating its therapeutic effect. Accordingly, in an embodiment, the obtaining of the immunomodulatory fitness status of the sample in step b) comprises:
I) determining at least one patient-reported outcome measure (PROM) score from an assessment of an MSC-treatable disease from a subject with the MSC-treatable disease pre treatment;
II) determining the at least one PROM score from the assessment of the MSC-treatable disease from the subject post treatment;
III) comparing the at least one PROM score in I) and II); and
IV) determining the immunomodulatory fitness of the sample based on the size of the improvement in the at least one PROM score post-treatment compared to pre-treatment, wherein an improvement in the at least one PROM score post-treatment is indicative that the immunomodulatory fitness of the sample is high, and wherein no improvement or a worsening in the at least one PROM score post-treatment is indicative that the immunomodulatory fitness of the sample is low.

Alternatively, the method of determining quantitative values for a set of CQAs for assessing immunomodulatory fitness of samples of MSCs does not comprise morphometric features such as cell diameter and/or cell circularity. Accordingly, in an embodiment, the method of a) comprises a.i. and/or a.ii.

Step II) can be performed at any suitable interval following the treatment with the sample, including 1 week, 2 weeks, 3 weeks, 4 weeks, 1 month, 2 months, 3 months, 4 months, 5 months, 6 months, 8 months, 10 months, 12 months, 18 months, 24 months, 3 years, 4 years, 5 years, 6 years, 8 years, 10 years, or longer following the cell-based anti-inflammatory treatment. Accordingly, in an embodiment, step II) is performed 12 or 24 months post treatment with the sample.

The term “MSC-treatable disease” as used herein means any disease known in the art wherein treatment with immunomodulatorily fit MSCs is clinically beneficial. The MSC-treatable disease can comprise an inflammatory disease, for example osteoarthritis, lupus, scleroderma, or rheumatoid arthritis. The MSC-treatable disease can also comprise graft versus host disease, stroke, inflammatory bowel disease or cardiac disease.

Accordingly, in an embodiment, the MSC-treatable disease comprises osteoarthritis, lupus, scleroderma, rheumatoid arthritis, graft versus host disease, stroke, inflammatory bowel disease, or cardiac disease.

In another embodiment, the MSC-treatable disease comprises osteoarthritis.

In yet another embodiment, the osteoarthritis comprises knee osteoarthritis.

The term “knee osteoarthritis” as used herein refers to osteoarthritis of the knee joint and can be characterized by radiographic changes to the cartilage and other tissues in this joint.

In an embodiment, the assessment of the MSC-treatable disease comprises an assessment of knee osteoarthritis. The assessment of knee osteoarthritis can comprise any method known in the art to assess knee osteoarthritis. In some embodiments, the assessment of knee osteoarthritis comprises Knee injury and Osteoarthritis Outcome Score (KOOS), Western Ontario and McMaster Universities Osteoarthritis Index (WOMAC), Visual Analogue Scale (VAS), Short Form 36 (SF-36) or Short Form 12 (SF-12), Tegner Lysholm Knee Score, Knee Society Clinical Rating System, Lequesne Index for Knee Osteoarthritis, Oxford Knee Score (OKS) and/or International Knee Documentation Committee (IKDC) Questionnaire.

In an embodiment, the assessment of knee osteoarthritis comprises KOOS and the PROM score comprises KOOS Pain, ADL, function in Sport and Recreation (Sports/Rec), knee-related quality of life (QOL), or Overall KOOS, or combinations thereof.

The term “administering” or “administration” as used herein refers to the placement of the MSC sample as disclosed herein into a subject by a method or route which results in at least partial delivery to a desired site. The MSC sample disclosed herein can be administered by any appropriate route which results in an effective treatment in the subject, and when the subject has knee osteoarthritis, can be by injection into the knee, for example by intra-articular injection. The MSC sample can also be administered, for example, by intravenous administration or intraosseous administration.

The term “treating”, “treatment”, and the like, as used herein, and as is well understood in the art, refers to an approach for obtaining beneficial or desired results, including clinical results. Beneficial or desired clinical results include, but are not limited to alleviation or amelioration of one or more symptoms or conditions, arresting development of disease, diminishment of extent of disease, stabilized (i.e. not worsening) state of disease, preventing spread of disease, delay or slowing of disease progression, amelioration or palliation of the disease state, including regression of the disease, diminishment of the reoccurrence of disease, and remission (whether partial or total), whether detectable or undetectable. “Treating” and “treatment” may also refer to prolonging survival as compared to expected survival if not receiving treatment.

Once values are obtained for a particular donor or CPPs, samples from the same donor or grown under the same CPPs can be evaluated. Accordingly, also provided herein is a method of determining immunomodulatory fitness of mesenchymal stromal cells (MSC) comprising
a) determining a sample critical quality attribute (CQA) profile, the sample CQA profile comprising:
   a. from the sample harvested in the presence of proinflammatory cytokines stimulation post-harvest: the mRNA levels of at least one, at least two, at least three, at least four, at least five, at least six, at least seven or all of the following genes: THBS1, CCN2, EDN1, ACTA2, PDCD1LG2, TNFAIP6, ANGPT1, CXCL8; and/or
   b. from the sample harvested in the absence of the proinflammatory cytokines stimulation post-harvest: the mRNA levels of at least one, at least two, at least three, at least four, at least five, at least six, at least seven, at least eight or all of the following genes: CCN2, TSG101, THBS1, PDGFA, VEGFA, EDIL3, ACTA2, ANGPT1, ANG and/or the level of TGF-beta protein; and/or
   c. from a single cell suspension of the sample post-harvest without pro-inflammatory cytokines stimulation: cell diameter and/or cell circularity; and
b) ranking the CQA profile of a) based on a comparison of minimum and maximum values determined by the method disclosed herein for the same particular donor using the same particular CPPs to determine the immunomodulatory fitness.

Similarly, also provided herein is a method of determining quantitative values for a set of CQAs for assessing angiogenic fitness of samples of mesenchymal stromal cells (MSC), wherein the MSC samples are from different donors and/or from samples grown using different critical processing parameters (CPPs), the method comprising:
a) determining the values of the set of CQAs for each sample; wherein the set of CQAs comprise:
   a. from the sample cultured and harvested:
      i. in the presence of proinflammatory cytokines post-harvest stimulation: the mRNA levels of at least one, at least two, at least three, at least four, at least five or all of the following genes: SMAD7, HGF, ANG, IDO1, TSG101, CXCL8 and/or the level of VEGF protein; and/or
      ii. in the absence of the proinflammatory cytokines post-harvest stimulation: the mRNA levels of the following genes: SOX9 and/or TNFAIP6 and/or the level of VEGF protein; and
b) testing each sample of MSCs for angiogenic fitness using in vitro assays or obtaining angiogenic fitness status of sample;
c) conducting a regression analysis between each sample CQA of a) with the angiogenic fitness of b); determining minimum and maximum data values of each CQA of the samples and using multivariate dimension reduction statistical or mathematical methods to assign minimization or maximization functions to indicate whether higher or lower values are desirable based on the directionality of correlation respectively.

Desirability analysis may be applied to analyze the profile of putative CQAs and to assign empirical rankings for donors and CPP conditions that result in desirable MSC angiogenic functionality.

Accordingly, in an embodiment, the method further comprises
d) assigning a weighting to each CQA for desirability analysis based on the R² values from the regression analysis; and
e) use of desirability analysis methods to assign a score from zero to one to each sample based on the range of data values; wherein zero is undesirable and one is highly desirable to obtain a set of values for MSCs for each donor grown using particular CPPs.

The testing for angiogenic fitness in step b) may be accomplished by an in vitro human umbilical vein tube formation assay. Other in vitro angiogenic assays are known in the art and may also be used, including, without limitation, measurement of MSC-mediated effects on endothelial cells (including matrix degradation, migration, proliferation, apoptosis and morphogenesis), assays for vessel outgrowth, and the chick chorioallantoic membrane assay.

Alternatively, the testing for angiogenic fitness in step b) may comprise evaluating angiogenic effects of MSC using preclinical animal models. Such animal models, include, without limitation, the Matrigel implant model, the dorsal air sac model, and critical limb ischemia models. In these models, MSCs are administered, and the angiogenic effects of the cells can be measured by histological techniques, biomarker analysis, or other efficacy readouts specific to the model.

Testing the MSCs for angiogenic fitness is not required where the angiogenic fitness is already known. Accordingly, in an embodiment, the angiogenic fitness status of the sample in step b) is obtained from a clinical sample for which the angiogenic fitness or efficacy of the MSCs were previously evaluated clinically. For example, where the clinical sample is from a clinical study or pre-clinical or other correlated read-outs.

Once values are obtained for a particular donor or CPPs, samples from the same donor or grown under the same CPPs can be evaluated. Accordingly, also provided herein is a method of determining angiogenic fitness of mesenchymal stromal cells (MSC) from a particular donor using particular CPPs comprising
a) determining a sample critical quality attribute (CQA) profile, the sample CQA profile comprising:
   a. from the sample harvested in the presence of proinflammatory cytokines stimulation post-harvest: the mRNA levels of at least one, at least two, at least three, at least four, at least five or all of the following genes: SMAD7, HGF, ANG, IDO1, TSG101, CXCL8 and/or the level of VEGF protein; and/or
   b. from the sample harvested in the absence of the proinflammatory cytokines stimulation post-harvest: the mRNA levels of the following genes: SOX9 and/or TNFAIP6 and/or the level of VEGF protein;
b) ranking the CQA profile of a) based on a comparison of minimum and maximum values determined by the method described herein for the same particular donor using the same particular CPPs to determine the angiogenic fitness.

The above disclosure generally describes the present application. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

### EXAMPLES

The following non-limiting examples are illustrative of the present disclosure:

### Example 1

### Methods

### MSC(AT) Isolation, Culture, and CPPs

Subcutaneous human adipose tissue was obtained external to the knee joint in patients undergoing knee arthroscopy or from abdominal lipoaspirate (REB #18-5480 and #18-6345, see Table 1 for summary of donor characteristics). MSC(AT) were isolated and expanded using MesenCult™-ACF Plus xeno-free and antibiotic-free growth medium (StemCell Technologies, Vancouver, Canada) on standard tissue culture polystyrene flasks coated with animal component-free cell attachment substrate (StemCell Technologies) according to the manufacturer's instructions. Cells were expanded in a standard incubator at 37° C. in 5% CO₂ under ambient air. For passaging, flasks at approximately 80% confluency were washed with PBS (Wisent, St-Bruno, Canada) and harvested using TrpLE (Gibco, Waltham, USA) prior to re-plating at 5,000 cells/cm². All experiments were performed using MSC(AT) between passage 3 and passage 5.

At approximately 70-80% confluence, MSC(AT) were transiently (16-20 h) cultured under varied CPP conditions, including 3D normoxic (3D-N), 2D hypoxic (2D-H), or 2D normoxic (2D-N) conditions. These culture steps were performed on separate flasks cultured in parallel using MesenCult™-ACF Plus medium supplemented with 1% (v/v) human serum album (HSA; Canadian Blood Services, Ottawa, Canada). 3D-N culture was performed by harvesting MSC(AT) from adherent flasks and plating cells on ultra-low attachment surfaces (Corning, Corning, USA) at 26,700 cells/cm2 and 200,000 cells/mL in medium supplemented with 2 ng/ml IL-6 (Peprotech, Cranbury, USA; used to support cell viability in the 3D culture), based on previously reported methods that allow spontaneous aggregation of MSCs into cell clusters [39]. Flasks from the same batch of cells were cultured in parallel under 2D-N (maintained in standard tissue culture incubator) or 2D-H conditions (38 mmHg 02, i.e., 5% O2 under standard atmospheric pressure) using a HypoxyLab workstation (Oxford Optronix, Milton, UK) for the same duration as 3D culture.

Prior to experiments, 3D cell aggregates were collected from ultra-low attachment flasks. Following multiple PBS washes of the flask and mixing, samples were removed for cell counting. Cell enumeration was performed after dissociating the aggregates using Accumax™ solution (Sigma, St. Louis, USA), according to previously published methods [40]. For MSC(AT) cultured under 2D-N and 2D-H conditions, flasks were washed with PBS and incubated in TrpLE solution, followed by neutralization with complete medium and cell counting. All cell counts were performed using the Vi-Cell XR Cell Counter (Beckman Coulter, Brea, USA). Prior to plating cells for experiments, excess PBS was added to cell suspensions to dilute residual growth factors/cytokines before centrifugation (350×g, 5 min) to pellet the cells.

### Morphometric and Surface Marker Characterization of MSC(AT)

Cell diameter and circularity was measured for single cell suspensions using the Vi-Cell XR Cell Counter (Beckman Coulter). 3D-N MSC(AT) were dissociated into single cell suspensions as described above. To analyze maximum feret diameters and circularity of whole intact 3D-N MSC(AT) aggregates, 10× phase-contrast images were captured using an EVOS XL Core Cell Imaging System (ThermoFisher, Waltham, USA). A semi-automated algorithm was developed in ImageJ [41] based on rolling ball subtraction to create binary images for particle analysis, and a minimum of 230 aggregate measurements were performed per donor and condition.

Surface marker expression of MSC(AT) was measured following previously established protocols [8] and in accordance with IFATS/ISCT guidelines (positive marker threshold: >80%, negative marker threshold: <2%) [42]. The following PE-conjugated anti-human antibodies from BioLegend (San Diego, USA) were used: anti-CD90 (cat. 328109), anti-CD73 (cat. 344004), anti-CD44 (cat. 338807), anti-CD29 (cat. 303003), anti-CD13 (cat. 301703), anti-CD34 (cat. 343506), anti-CD31 (cat. 303105), anti-CD45 (cat. 304008), and anti-CD105 (cat. 323205). For staining, single cell suspensions of 2D-H and 2D-N MSC(AT) were obtained by TrpLE dissociation, while 3D-N aggregates were digested using Accumax™ solution as described above. To evaluate the effects of Accumax™ digestion on the surface marker profile of 2D MSC(AT), a subset of 2D-N MSC(AT) were digested using the same Accumax™ digestion protocol as used for 3D cell aggregates. Samples were characterized using the FC500 flow cytometer (Beckman Coulter) and analyzed using FlowJo version 10 software (Ashland, USA).

### Western Blotting

Western blot analysis was performed to confirm CD105 expression in MSC(AT) cultured under varying CPP conditions. Cells were lysed using a buffer containing 50 mM Tris-HCl (pH 7.5), 1 mM EGTA, 1 mM EDTA, 1% (w/v) Nonidet P40, 1 mM sodium orthovanadate, 50 mM sodium fluoride, 5 mM sodium pyrophosphate, 0.27 M sucrose, and a protease inhibitor cocktail (Roche). Protein concentration of all samples was measured using the Pierce BCA protein assay (ThermoFisher). Protein samples were loaded in a 10% polyacrylamide gel (20 μg/well) for electrophoresis followed by transfer to nitrocellulose membranes. Membranes were then blocked with TBS-T containing 5% (w/v) BSA and they were immunoblotted in the same buffer overnight at 4° C. with an anti-CD105 primary antibody (cat. 323205, BioLegend, 1:1,000 dilution), or for 2 h with an anti-ß-actin antibody (Sigma, 1:10,000 dilution) used for the loading control. Washes were then performed with TBS-T and the blots were then incubated with secondary HRP-conjugated antibodies in 5% skimmed milk. The blots were washed in TBS-T and the signal was detected with the enhanced chemiluminescence reagent (ECL; GE Healthcare, Chicago, USA) and using a chemiluminescent imaging system (Bio-Rad, Hercules, USA).

### Gene Expression and Soluble Factor Measurements

After harvesting cells from 2D-N, 2D-H, and 3D-N conditions, cells were added to 24-well plates at 60,000 cells/well in MesenCult™-ACF Plus medium supplemented with 1% HSA (v/v) with or without addition of pro-inflammatory licensing cytokines. The licensing cytokines (all purchased from Peprotech) consisted of IFNγ (30 ng/ml), TNFα (10 ng/ml), and IL-1B (5 ng/ml). Cells harvested from 2D-N or 2D-H conditions were maintained under normoxic or hypoxic (38 mmHg O2) conditions for 24 h, respectively, while cell aggregates from the 3D-N condition were plated on ultra-low attachment 24-well plates (Corning) without dissociation. After the culture period, conditioned medium was collected, centrifuged (1,000×g, 5 min) and frozen at −80° C. The remaining cells were washed in PBS and RNA was extracted using a RNeasy Mini kit (Qiagen, Hilden, Germany) according to the manufacturer's instructions. RNA concentration and purity was measured using a DS-11 Spectrophotometer (DeNovix, Wilmington, USA).

The nCounter platform (NanoString, Seattle, USA) was used as a highly sensitive tool that detects target mRNAs with high specificity and without amplification. Samples (100 ng RNA/sample) were run on the nCounter MAX Analysis system (St. Michael's Genomics Molecular Biology Core facility) according to the manufacturer's instructions using a custom CodeSet 58-gene panel. The data was processed using the nSolver version 4.0 software (NanoString) according to the manufacturer's instructions to obtain mRNA counts normalized to the synthetic positive control probes and to reference genes. The following were measured as potential reference genes: ABCF1, GAPDH, GUSB, HPRT1, LDHA, RPL19, RPLP0, TUBB, POLR1B, and TBP. Reference gene stability was evaluated using geNorm analysis in the nSolver software; POLR1B and TBP were subsequently discarded as reference genes and not used for normalization. Normalized mRNA counts below 20 were assigned values of 1 if >33% of samples were within range. The following genes were undetectable (<33% of samples were within range) under both licensed and unlicensed conditions: ANGPT2, BDNF, BMP7, CCR7, CD200, CTLA4, CXCR4, IGF1, IL10, IL12A, NGF, PDGFB, PROK1, and CXCL12. Under licensed conditions, VASH1 and SOX9 were undetectable. Under unlicensed conditions, CD274, IDO1, NFKBIA, NOS2, and PDGFA were undetectable. A broad spectrum of MSC(AT) transcripts were selected initially but many of the undetectable genes are not commonly or are inconsistently expressed by MSCs. Other genes may have tissue of origin-dependent or context-dependent expression. All NanoString data has been deposited in NCBI's Gene Expression Omnibus [43] accessible through GEO Series accession number GSE212368.

Conditioned medium samples were analyzed for soluble factors using a custom 10-analyte LEGENDplex immunoassay (BioLegend) according to the manufacturer's instructions, and samples were run on a FACSCanto™ II flow cytometer (BD Biosciences, Franklin Lakes, USA). Values below the assay limit of detection were imputed as half of the lower limit of detection if >33% of samples were within range. IL-10, PIGF, and PD-L1 were undetectable in the samples tested. IL-1RA was undetectable in unlicensed samples only.

Pilot gene expression experiments were performed to i) examine gene expression in MSC(AT) cultured using the combination of 3D and hypoxic culture (3D-H), and ii) to evaluate effects of IL-6 treatment on MSC(AT) cultured under 2D-N conditions using the same IL-6 concentration and treatment duration as used for 3D-N conditions. Both sets of pilot experiments were performed under licensed conditions using the methods outlined above. After licensing, RNA was isolated from MSC(AT) by Trizol-chloroform extraction and cDNA was generated using SuperScript™ IV VILO™ Master Mix (Invitrogen, Waltham, USA). qPCR was run using custom primers (Table 5, Invitrogen) and FastStart Universal SYBR Green Master Mix (Roche, Basel, Switzerland) on a QuantStudio™ 5 system (ThermoFisher). Results were normalized (ΔΔCT) against reference genes (B2M and RPL13A) and presented as fold-change values relative to 2D-N culture conditions.

### In Vitro Mϕ Polarization

Peripheral blood-derived CD14+ monocytes were isolated from a leukopak (StemCell Technologies) by Ficoll density gradient separation and selection with CD14+ magnetic beads as previously described [44]. Cryopreserved monocytes were thawed and plated on 24-well plates at 100,000 cells/well and allowed to acclimate for 48 h in co-culture medium consisting of: 1 mM sodium pyruvate (Gibco), 1% penicillin-streptomycin (Gibco), 10% FBS (Wisent), and 10% low-glucose DMEM (Sigma) in RPMI medium (Gibco). Prior to co-culture, MSC(AT) were harvested from 2D-N, 2D-H, and 3D-N conditions, plated on 0.4 μm transwell inserts at 10,000 cells/insert in a separate 24-well plate, and allowed to attach for 2 h. The inserts were then transferred to MO wells and co-cultured for 20-24 h. Transwell inserts containing MSC(AT) were then removed, and lipopolysaccharide was spiked into wells at a final concentration of 2.5 ng/ml. After a 4 h incubation, Mos (both adherent and in suspension) were collected and stored in Trizol (Roche) at −80° C. Conditioned medium was also collected and stored at −80° C.

Levels of TNFα in conditioned medium were measured by ELISA (R&D Systems, Minneapolis, USA) according to the manufacturer's instructions. To analyze Mϕ gene expression, RNA was isolated by Trizol-chloroform extraction and cDNA was generated using SuperScript™ IV VILO™ Master Mix (Invitrogen). qPCR was run using custom primers (Table 5, Invitrogen) and FastStart Universal SYBR Green Master Mix (Roche) on a QuantStudio™ 5 system (ThermoFisher). Results were normalized (ΔΔCT) against reference genes (ACTB, B2M, and TBP) and presented as fold-change values relative to Mϕ cultured without MSC(AT) (SOLO condition).

### In Vitro HUVEC Tube Formation

To prepare conditioned medium for the HUVEC tube formation assay, MSC(AT) were harvested from 2D-N, 2D-H, and 3D-N conditions, plated at 15,000 cells/cm2 and 100,000 cells/mL of growth medium (no added HSA or exogenous cytokines), and incubated for 24 h in cell culture incubators. MSC(AT) harvested from all three CPP conditions were maintained under these conditions to prepare conditioned medium. To prepare unconditioned medium controls, growth medium was incubated on a cell culture plate for the same duration. Following the 24 h incubation period, the conditioned medium was collected, centrifuged (1,000×g, 5 min), and the supernatant was frozen at −80° C. for future use in the HUVEC tube formation assay.

Five to seven days prior to the tube formation assay, P4 HUVECs (Cat. CC2519, Lonza, Basel, Switzerland) were thawed and expanded in EGMTM-2 (Lonza) according to the manufacturer's instructions. The HUVEC tube formation assay was performed based on previously published methods [38]. 96-well plates were pre-coated with Cultrex Reduced Growth Factor Basement Membrane Extract, PathClear (R&D Systems) according to the manufacturer's instructions. HUVECs were harvested and plated on the coated plates at 42,500 cells/cm2 in conditioned medium. The following medium formulations were used as controls: unconditioned medium, EGMTM-2 medium (positive control), and basal medium without addition of growth factors/supplements (negative control). All wells were imaged 6 h after plating the cells using the EVOS XL Core Cell Imaging System (ThermoFisher) at 4× objective. The images were analyzed using the Angiogenesis Analyzer plugin in ImageJ [45].

### Statistical Analysis

Plots were created using GraphPad Prism 6.0 (La Jolla, USA) and JMP Pro 14 (Cary, USA) software. All statistical tests are specified in the figure captions. One- and two-way ANOVA, as well as simple linear regression was performed using GraphPad Prism software. Unbiased hierarchical clustering (Ward method), principal component (PC) analysis (default estimation method), and desirability profiling was performed using JMP software. Analysis of differential gene expression was performed in nSolver (NanoString) using single linear regressions for each covariate (i.e., gene) with false discovery rate (FDR)-corrected p values calculated using the Benjamini-Yekutieli method [46]. Data were considered statistically significant based on a threshold of p<0.05. For functional in vitro assays (Mϕ polarization and HUVEC tube formation) where PC analysis was performed on the assay readouts, the PC1 score was taken as a ‘composite functional score’, based on previously published methods [14]. For linear regression, analysis between functional PC1 scores and MSC(AT) characteristics (genes, soluble factors, and morphometric features), statistically significant (p<0.05) correlations are reported along with correlations with 0.05<p<0.1 which were considered as near-significant. Normal residuals were checked to fulfill assumptions of linear regression, and parametric correlations were performed independently for each individual MSC(AT) characteristic that was measured.

Desirability profiling [47] was used as a tool for multiple response optimization that assigns individual responses a score from zero to one based on the range of data values, with zero representing an undesirable response, and one representing a highly desirable response. Minimization or maximization functions were assigned to each MSC(AT) characteristic (including genes, soluble factors, and morphological features) to indicate whether higher or lower values were desirable, based on outcomes from linear regression analyses with functional PC1 scores. Based on the regression analyses, all statistically significant (p<0.05) and near-significant (p<0.1) MSC(AT) characteristics were included in the desirability analysis. The R² values from the regression analyses were applied as weightings for each MSC(AT) characteristic.

### Results

### An In Vitro Assay Matrix of Readouts to Identify Putative CQAs

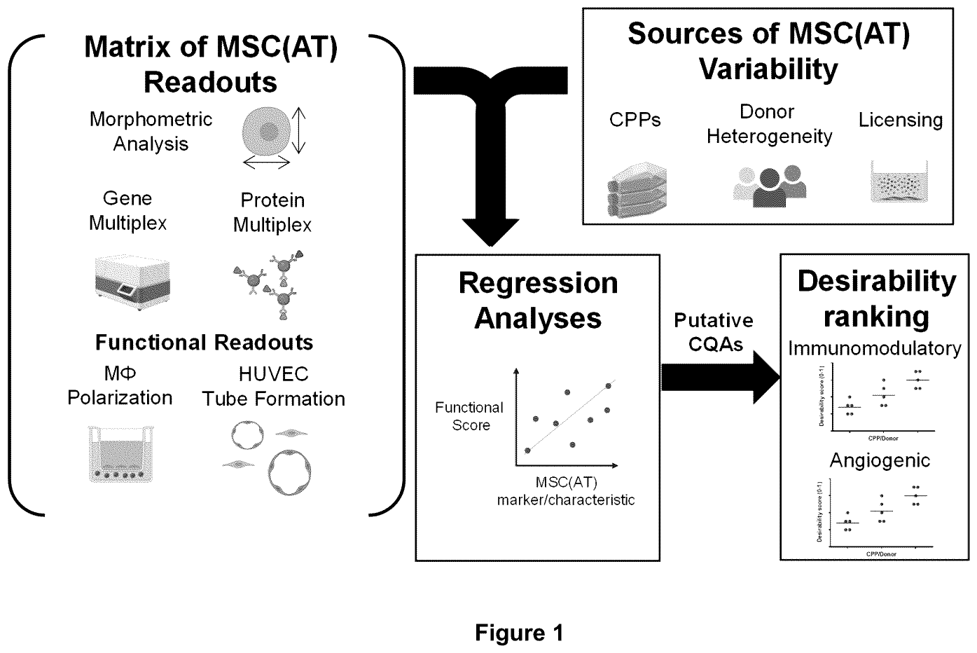

A matrix of in vitro readouts was used to investigate the responses of putative CQAs to varying donors and CPPs that were specifically selected for their known ability to enhance MSC immunomodulatory and angiogenic properties. The matrix consisted of multivariate morphometric measurements, gene expression, soluble factor analysis, and functional immunomodulatory and angiogenic readouts (FIG. 1). The matrix of readouts demonstrated sensitivity to multiple sources of MSC(AT) variability, including variations in select CPPs, donor heterogeneity, and licensing with pro-inflammatory cytokines. Linear regression analyses were used to refine putative CQAs by evaluating correlations with anchor functional in vitro immunomodulatory and angiogenic outcomes. A range of values was further provided for assessing basal MSC(AT) fitness range according to the significant and near-significant putative CQAs identified from the regression analyses. Desirability analysis was then applied to analyze the profile of putative CQAs and to assign empirical rankings for donors and CPP conditions that result in desirable MSC(AT) immunomodulatory and/or angiogenic functionality.

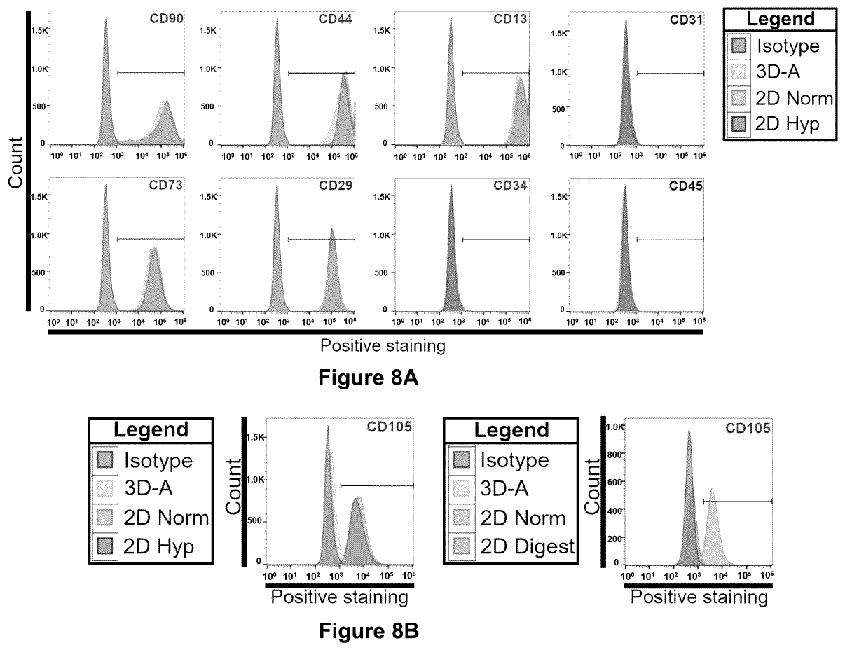

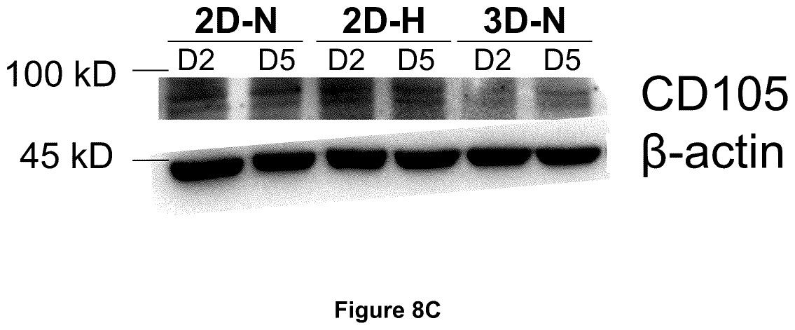

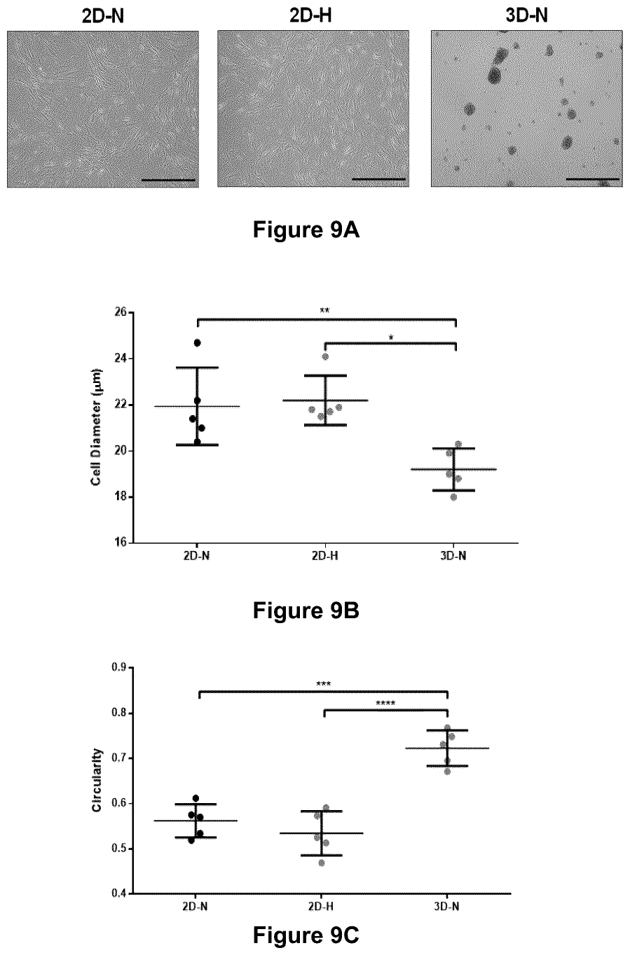

The CPPs investigated included 2D-N, 2D-H, and 3D-N culture conditions. These CPPs were chosen based on previous literature demonstrating that 3D and 2D hypoxic culture can enhance the immunomodulatory and angiogenic potency of MSCs [28,29,31,32,35]. MSC(AT) cultured under 2D-N, 2D-H and 3D-N conditions satisfied surface marker expression criteria [42] and were CD90+CD73+CD44+CD13+CD34-CD31-CD45-(Table 2). Notably, CD105 appeared to be cleaved under enzymatic conditions required for dissociating 3D cell aggregates for flow cytometry analysis (Table 2). Western blot analysis verified CD105 expression by MSC(AT) cultured using 3D-N conditions, albeit at lower levels relative to 2D-N and 2D-H conditions (FIG. 8). MSC(AT) cultured under 2D-N and 2D-H conditions displayed a characteristic spindle-like morphology, while MSC(AT) cultured under 3D-N conditions formed cell aggregates of varying sizes (FIG. 9A). Cell aggregates in the 3D-N condition had a median ferret diameter of 37.82 μm (range: 12.38-269.40 μm) and median circularity of 0.56 (range: 0.044-0.97). Morphometric measurements of MSC(AT) revealed differences in cell morphology with changes in CPP conditions. Analysis of single cell suspensions obtained from each culture condition demonstrated significantly reduced diameter and greater circularity of 3D-N MSC(AT) relative to 2D-N and 2D-H MSC(AT) (FIG. 9B,9C).

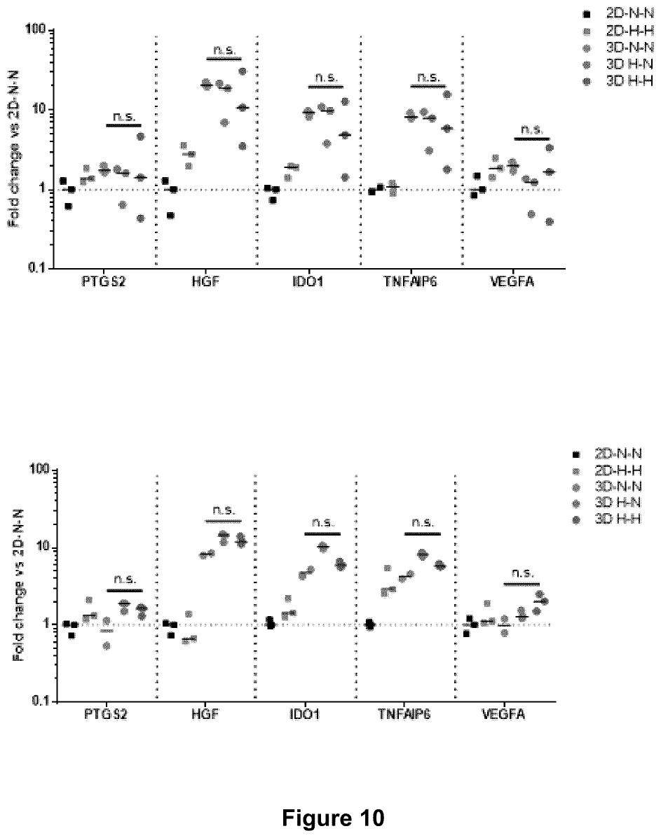

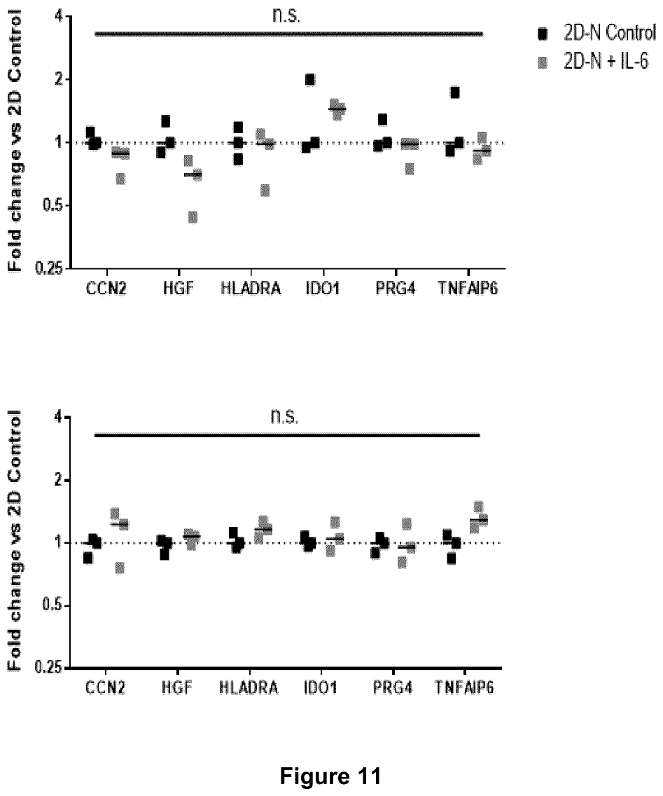

For gene expression and soluble factor analysis, 2D-N, 2D-H, and 3D-N MSC(AT) were subject to pro-inflammatory licensing conditions (with a cocktail of three pro-inflammatory cytokines, TNFα, IFNγ, and IL-1β to simulate a wide range of disease conditions) or cultured under unlicensed conditions in the absence of pro-inflammatory stimuli. The combination of 3D and hypoxic culture (3D-H) was investigated by gene expression analysis using an abbreviated panel of anti-inflammatory/angiogenic markers measured in two MSC(AT) donors (FIG. 10) and demonstrated no significant benefit of combining these culture conditions. Thus, 3D-N was used as the select CPP in all subsequent experiments. The effects of IL-6 (used to support cell viability under the 3D-N culture condition) were also evaluated on MSC(AT) cultured under 2D-N culture conditions using the same cytokine concentration as used for the 3D-N culture method. An abbreviated panel of genes was selected using markers that were significantly differentially expressed in the 3D-N culture condition relative to 2D-N culture. Gene expression analysis revealed no significant effect of IL-6 on 2D-N culture (FIG. 11), suggesting that the 3D geometry was the major factor that primed the cells rather than IL-6.

Curated Gene Expression and Soluble Factor Profiles are Differentially Sensitive to Donor Heterogeneity and Select CPPs that Enhance MSC Potency

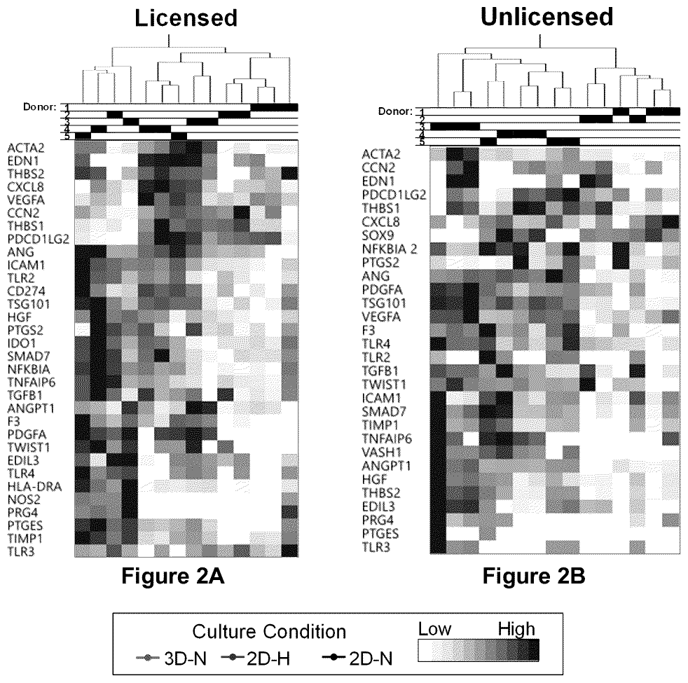

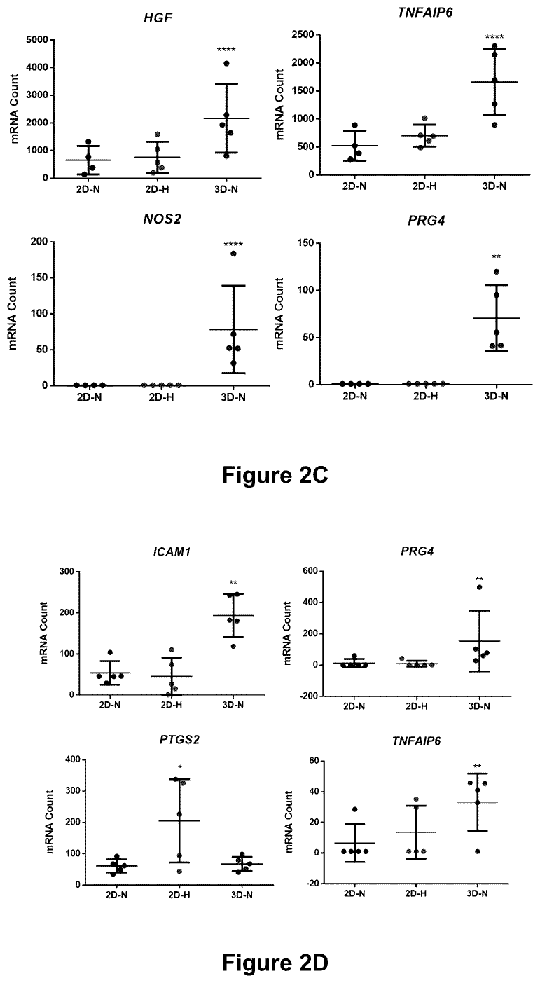

Gene expression analysis was performed using a curated panel of markers including predominantly immunomodulatory and angiogenic genes (data provided in Tables 6, 7, 8, and 9). The panel was selected based on previous literature [11,17,48,49] and experience using MSC(M) in an osteoarthritis clinical trial [8]. It was used to evaluate MSC(AT) fitness across multiple donors and while modulating select CPP conditions associated with enhanced immunomodulatory and angiogenic functionality. Unbiased hierarchical clustering analysis revealed that MSC(AT) gene expression profiles clustered according to both variations in CPP conditions and donor heterogeneity (FIG. 2A,2B). Under licensed conditions, CPP variations, specifically 3D-N configurations of culturing MSC(AT) clustered separately from 2D-N and 2D-H cultures for all but one donor (Donor 1) which clustered together regardless of CPP variations. For MSC(AT) cultured under 2D-N and 2D-H conditions, the gene expression profiles clustered together by donor rather than oxygen tension under licensed conditions. Interestingly, under unlicensed conditions, gene expression profiles clustered primarily by donor, regardless of variations in CPPs. Overall, variations in CPP conditions by changing culture geometry or oxygen tension and donor heterogeneity resulted in shifts in gene expression profiles dependent on pro-inflammatory licensing conditions or unlicensed conditions. For example, genes such as HGF (multifunctional growth factor) and TNFAIP6 (anti-inflammatory gene encoding TSG6), were upregulated by 3D-N culture conditions relative to 2D-N culture conditions, under licensed conditions (FIG. 2C). NOS2 (encoding for inducible nitric oxide synthase, iNOS which may indicate cell stress or enhanced immunosuppressive functions for mouse, but not human MSCs [50]) and PRG4 (lubricating proteoglycan with potential immunomodulatory functions [51,52]) were also upregulated under these conditions. ICAM1 (immunomodulatory marker), PRG4, and TNFAIP6 were upregulated by 3D-N culture conditions relative to 2D-N under unlicensed conditions (FIG. 2D). Furthermore, 2D-H culture conditions induced augmented expression of the immunomodulatory marker PTGS2 relative to 2D-N culture conditions under unlicensed settings.

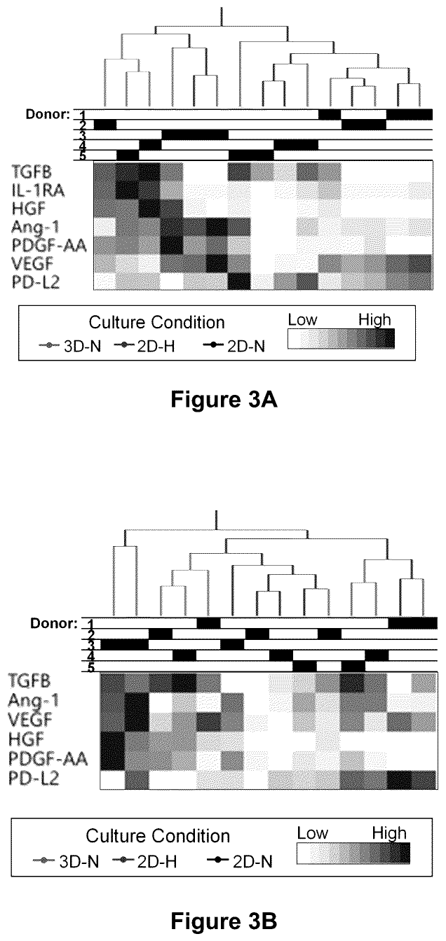

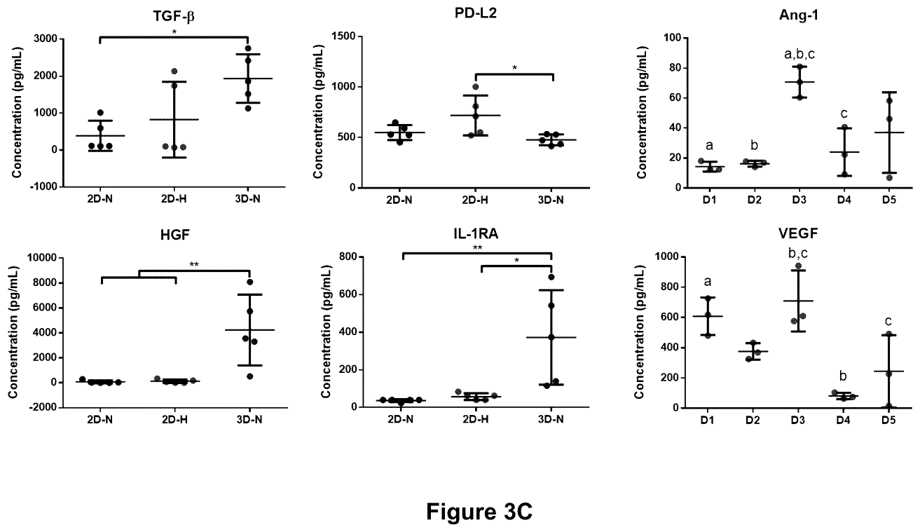

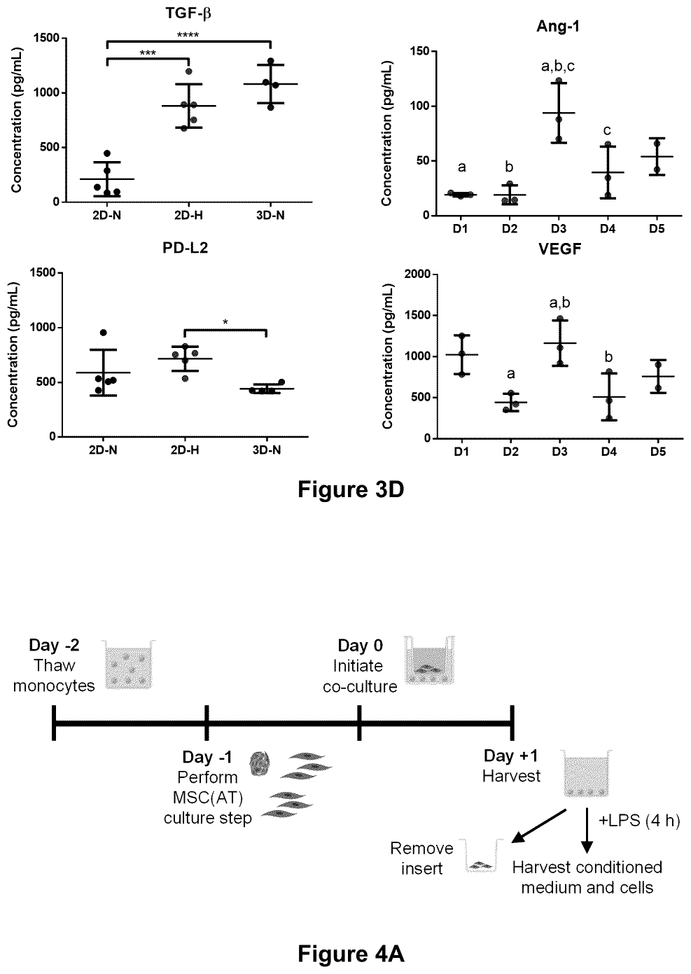

Levels of soluble factors in conditioned medium were queried using a curated sub-panel of immunomodulatory and angiogenic factors, based on significant results from the gene expression analysis (soluble factor data provided in Tables 10, 11). Unbiased hierarchical clustering demonstrated that soluble factor profiles clustered based on both donor heterogeneity and variations in CPP conditions under licensed and unlicensed conditions (FIG. 3A,3B). Analysis of individual soluble factors was performed to evaluate the effects of CPP and donor heterogeneity on each soluble factor. Under licensed conditions, MSC(AT) cultured in 3D-N configurations expressed significantly higher levels of the multifunctional cytokine TGF-β relative to 2D-N, and significantly lower levels of the immunomodulatory factor soluble PD-L2 relative to 2D-H (FIG. 3C). Furthermore, MSC(AT) cultured in 3D-N configurations expressed significantly higher levels of the growth factor HGF and the immunosuppressive factor IL-1RA relative to both 2D-N and 2D-H MSC(AT) under licensed conditions. Under unlicensed conditions, 2D-H and 3D-N MSC(AT) expressed significantly higher levels of TGF-β relative to 2D-N, while soluble PD-L2 was significantly upregulated by 2D-H relative to 3D-N MSC(AT) (FIG. 3D). Expression of the angiogenic markers angiopoietin-1 (Ang-1) and VEGF showed statistically significant differences between donors, with Donor 3 expressing the highest levels of both factors under both licensed and unlicensed conditions (FIG. 3C,3D). Taken together, the curated panel of genes and soluble factors were differentially sensitive to donor heterogeneity and CPPs under licensed and unlicensed conditions. Interestingly, donor heterogeneity was masked under licensed conditions, and dominated under unlicensed conditions. Further, culturing MSC(AT) under 3D-N conditions rendered them with an elevated profile of anti-inflammatory/immunosuppressive genes (HGF, TNFAIP6, PTGES, PTGS2, TLR2, NFKBIA, TGFB1, PRG4, IDO, ICAM1, TLR4) and soluble factors (TGF-β, HGF, IL-1RA), corroborating previous reports that have investigated MSC culture in 3D formats [31,32,53].

### MSC(AT)-Mediated In Vitro Functional Polarization of Mϕ Readouts are Dependent on Donor Heterogeneity and CPPs

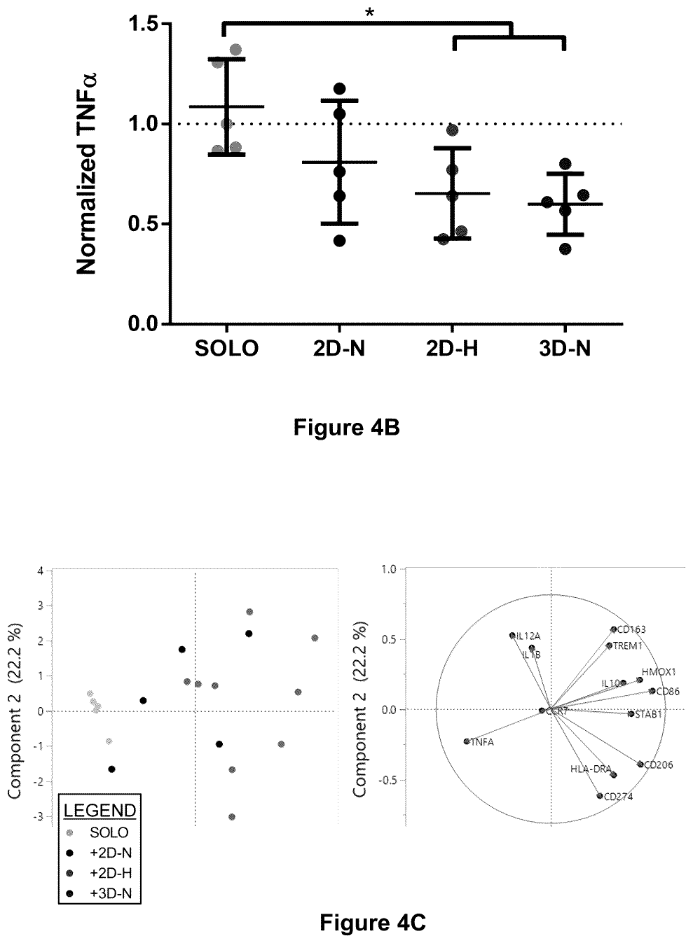

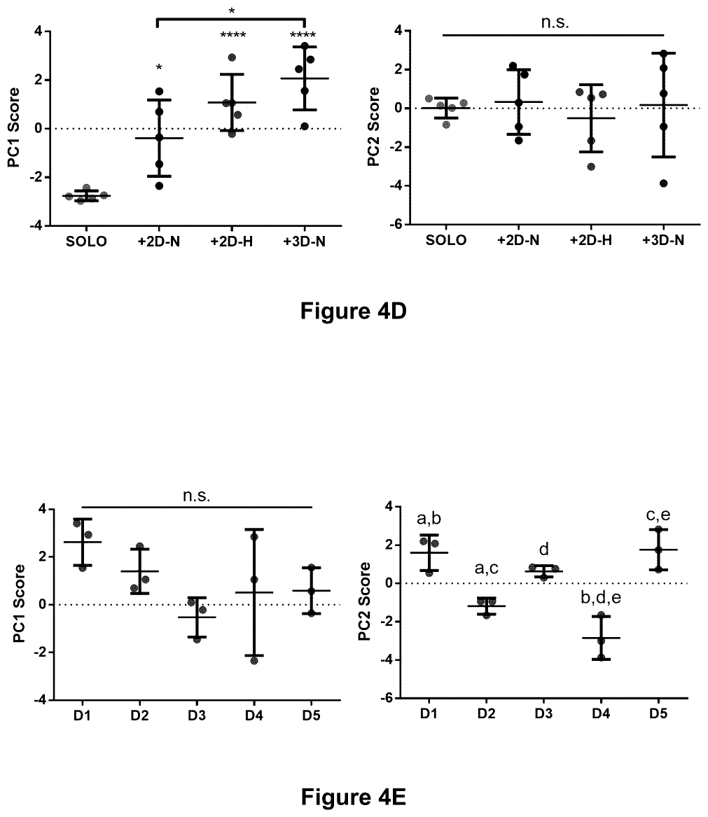

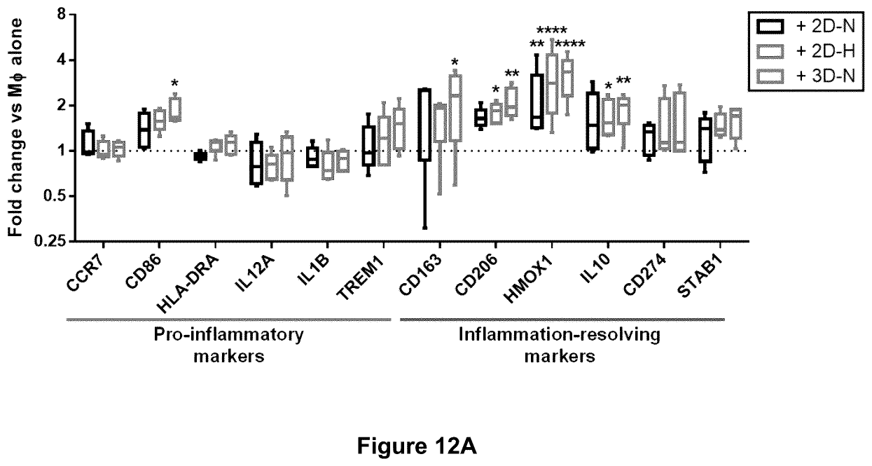

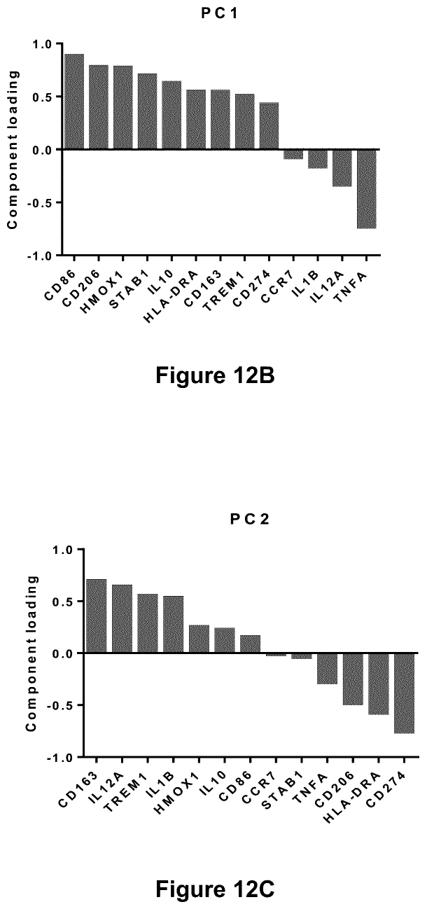

To further probe immunomodulatory properties of MSC(AT), an indirect co-culture assay was performed to evaluate functional Mϕ polarization (FIG. 4A). Alterations to CPP conditions by changing MSC(AT) culture configuration (3D-N) or oxygen tension (2D-H) significantly reduced levels of pro-inflammatory TNFα in conditioned medium relative to Mϕs alone (FIG. 4B), suggesting that these culture conditions enhanced anti-inflammatory functions of MSC(AT). Gene expression analysis of Mϕs revealed statistically significant increased expression of inflammation-resolving Mϕ markers (CD206, HMOX1, and IL10) in co-cultures with MSC(AT) in 3D-N and 2D-H culture conditions relative to MP alone (FIG. 12A). In addition, MSC(AT) cultured in 3D-N conditions significantly upregulated Mϕ expression of CD86 (pro-inflammatory) and CD163 (inflammation-resolving), while 2D-N culture conditions significantly upregulated expression of HMOX1 only. PC analysis was applied as an unbiased dimension reduction tool to evaluate the full gene expression panel and TNFα protein levels (FIG. 4C, loading plots for PC1 and PC2 displayed in FIG. 12B, 12C). Increased expression of CD86, CD206, HMOX1, and STAB1 genes, and reduced expression of TNFα protein were the main contributors to higher scores along the PC1 axis (accounting for 35.7% of variation). Reduced expression of CD274 and HLADRA, and increased expression of CD163, IL12A, and TREM1 genes drove higher scores along the PC2 axis (accounting for 22.2% of variation).

To further probe the results of the PC analysis, individual PC1 and PC2 scores were plotted for each CPP and donor combination as these scores capture multivariate heterogeneity in a reduced, single dimension with PC1 capturing a larger proportion of the total existing variation in the dataset relative to PC2. Analysis of individual PC1 scores demonstrated that separation along the PC1 axis was driven by co-culture with MSC(AT) as indicated by statistically significant differences in PC1 scores for MSC(AT) cultured under 2D-N, 2D-H, and 3D-N conditions relative to solo Mϕ (positive control) without MSC(AT) (FIG. 4D). Given that the PC1 axis accounted for differences in Mϕ phenotypic marker profiles relative to positive control, PC1 scores were considered as a ‘composite functional score’ [14] for MSC(AT) immunomodulatory function. With the exception of CD86 gene expression, higher scores along the PC1 axis were generally associated with greater inflammation-resolving Mϕ polarization as evidenced by increased expression of CD206, HMOX1, and STAB1, along with reduced expression of TNFα protein. Using PC1 scores as an analytic, variations in CPP conditions resulted in differences in MSC(AT)-mediated Mϕ polarization toward inflammation-resolving subtypes. Notably, MSC(AT) cultured in 3D-N configurations displayed the highest PC1 scores, and these were significantly different from 2D-N MSC(AT), suggesting that 3D-N MSC(AT) displayed a superior capacity to polarize Mϕ phenotype toward inflammation-resolving subtypes. No significant differences in culture conditions were observed along the PC2 axis. Analysis of PC1 scores across individual donors showed no significant differences (FIG. 4E); while not significant, Donor 1 displayed the highest PC1 scores suggesting that Donor 1 may have intrinsic improved immunomodulatory basal functionality, and Donor 3 displayed the lowest scores. In contrast, a significant effect of donor heterogeneity was observed along the PC2 axis. Given that PC2 scores represent changes in both pro-inflammatory and inflammation-resolving Mϕ markers, these data suggest that donor heterogeneity dictates MSC(AT)-mediated polarization of Mϕ toward mixed phenotypes.

### In Vitro MSC(AT)-Mediated Functional Angiogenesis Readouts are Dependent on Donor Heterogeneity and CPPs

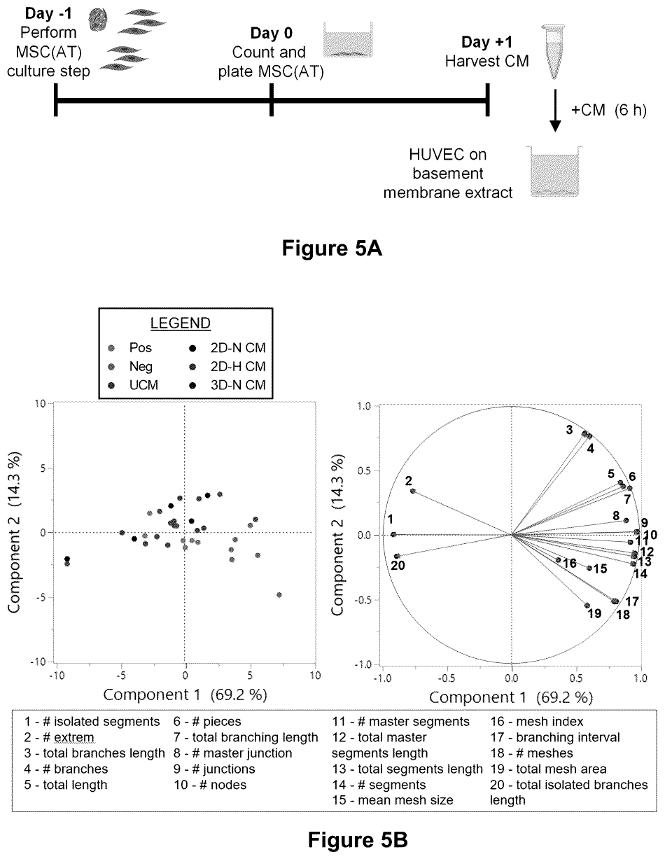

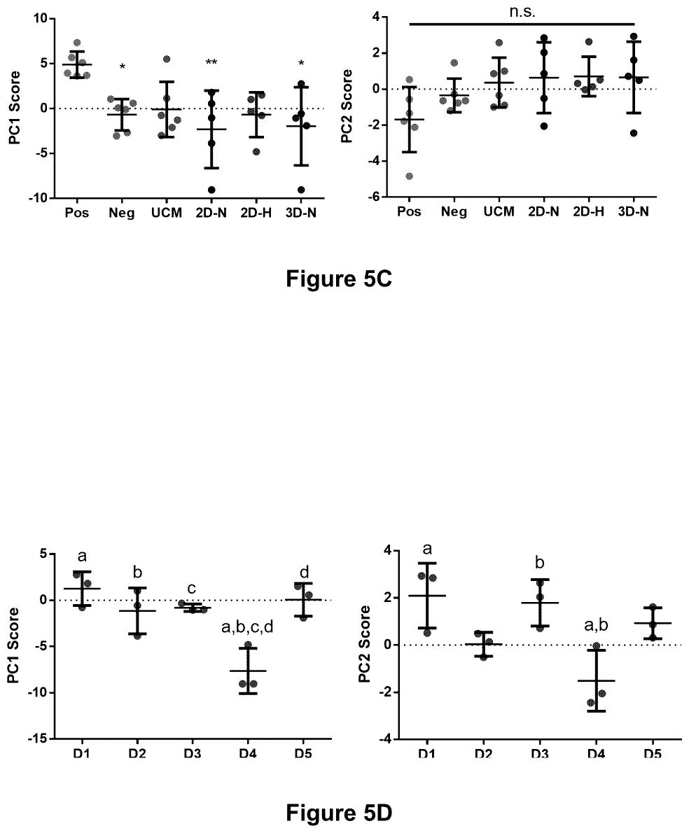

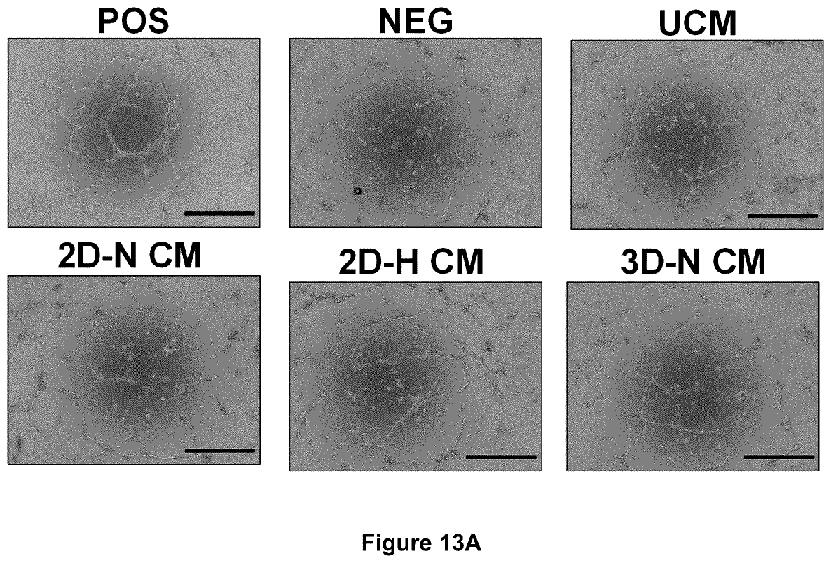

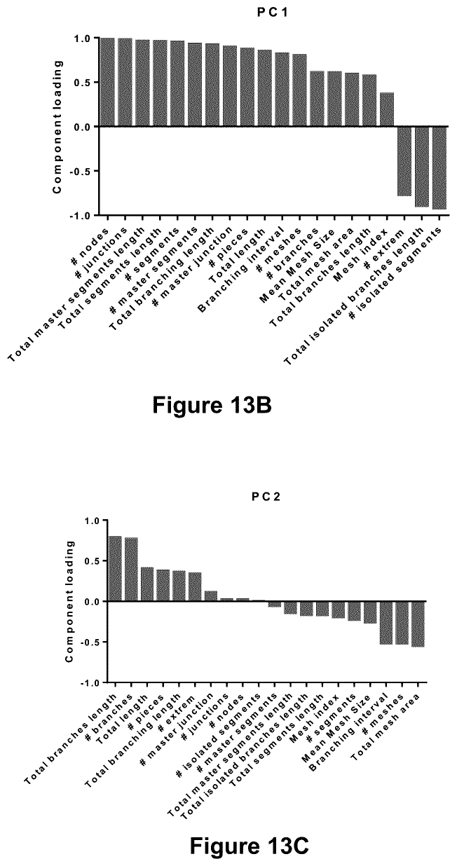

To evaluate the angiogenic functions of MSC(AT), the effects of MSC(AT) conditioned medium on HUVEC tube formation was explored (FIG. 5A, FIG. 13A). Recognizing that there is no standard quantitation method for HUVEC tube formation and that different measurements can yield different insights into in vitro angiogenesis [54], the ImageJ Angiogenesis Analyzer plugin was employed which provides twenty different types of measurements [45]. PC analysis was applied to analyze all twenty parameters that profile tube formation image analyses (FIG. 5B, loading plots for PC1 and PC2 displayed in FIG. 12B, 12C). An increased number of nodes, number of junctions, and the total master segment length were the main contributors to higher scores along the PC1 axis (accounting for 69.2% of variation), indicative of greater angiogenesis. Increased total branches length and number of branches, along with reduced total mesh area drove higher scores along the PC2 axis (accounting for 14.3% of variation). Analysis of individual PC1 scores demonstrated that the positive control group (HUVECs cultured in pro-angiogenic medium) displayed significantly higher PC1 scores relative to the negative control (HUVECs cultured in basal medium), and to HUVECs cultured in conditioned medium derived from 2D-N or 3D-N MSC(AT) culture conditions (FIG. 5C). As above, the PC1 scores were considered as a ‘composite functional score’ for MSC(AT) angiogenic functions given that higher scores along this axis reflected greater HUVEC tube formation. Surprisingly, conditioned medium derived from MSC(AT) cultured under 2D-H or 3D-N conditions did not elicit significant increases in HUVEC PC1 scores, and there were no significant differences between PC1 scores across MSC(AT) by varying CPP conditions. PC1 scores were instead driven by MSC(AT) donor differences (FIG. 5D). Donors 1, 2, 3, and 5 all displayed statistically significant higher scores relative to Donor 4 indicative of greater angiogenic function. Analysis of PC2 scores similarly showed no significant effect of the conditioned medium from the different groups representing variability in culture conditions, or the controls on HUVEC tube formation profiles (FIG. 5C). Some variation along the PC2 axis was driven by donor with Donors 1 and 3 displaying significantly higher PC2 scores relative to Donor 4 (FIG. 5D).

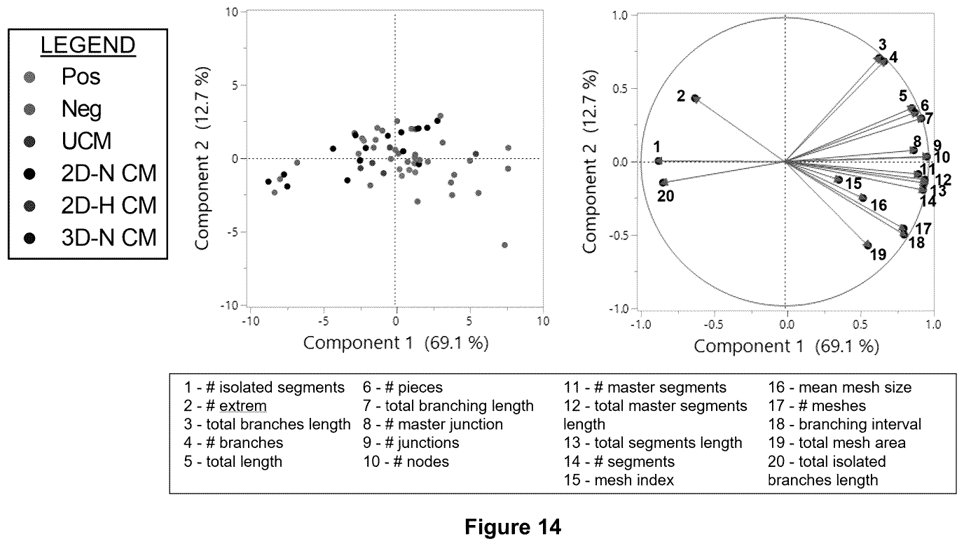

To further analyze effects mediated by variations in donor and experimental batches, PC analysis was performed on all biological and technical replicates (FIG. 14) and PC1 scores were investigated for each donor (Table 12 and Table 13). Under this analysis, conditioned medium derived from MSC(AT) cultured under 2D-H conditions had the highest mean HUVEC PC1 scores for four out of five donors (Donor 2, 3, 4, and 5) compared to scores for the 2D-N and 3D-N conditions. Taken together, the data suggests that donor heterogeneity dominated over effects mediated by variations in CPPs in the analysis of angiogenic readouts. The net effect of CPPs on HUVEC tube formation was variable, donor-dependent, and in part driven by CPP conditions that favour angiogenesis.

Generating a Matrix of Putative MSC(AT) CQAs Based on Correlation of Select Genes, Soluble Factors, and Morphological Features with Functional Immunomodulation

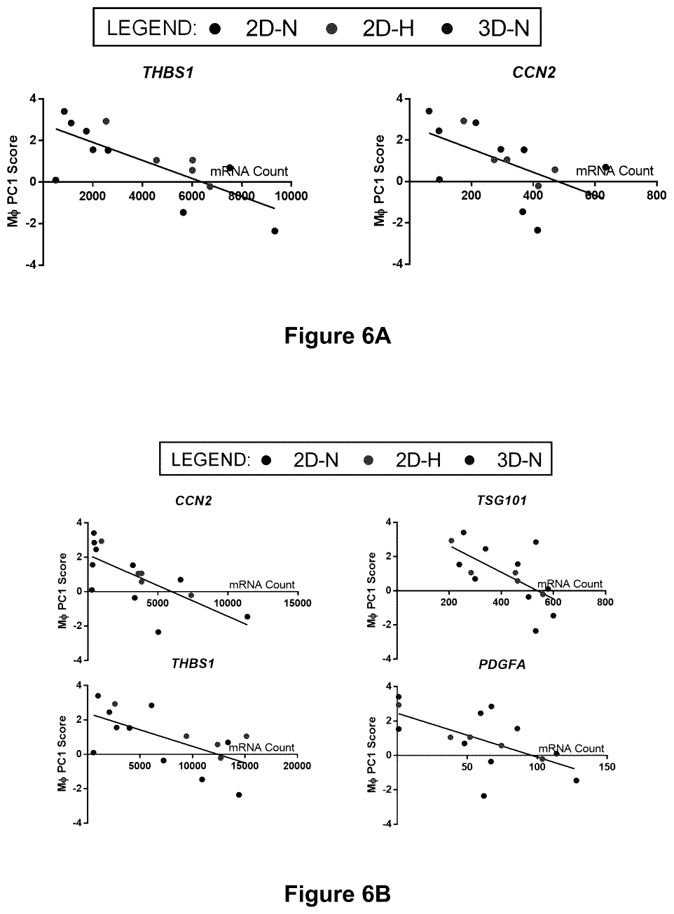

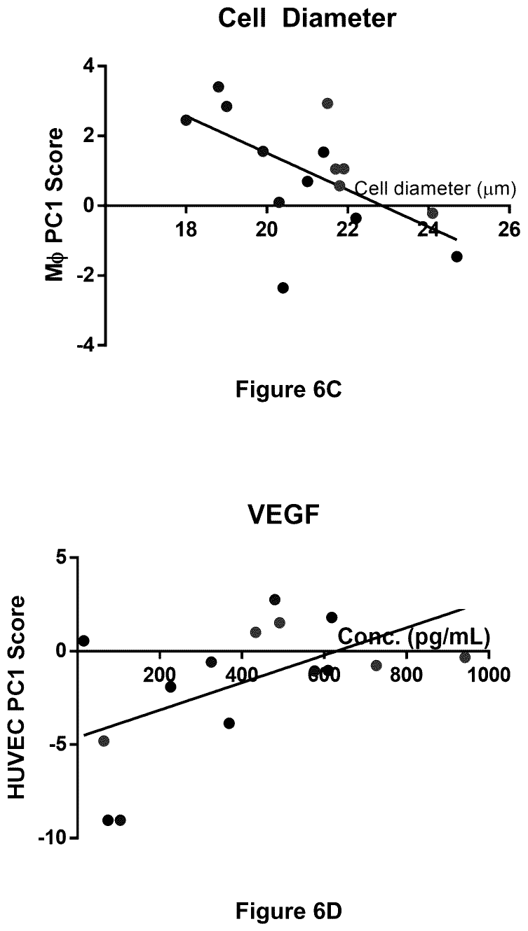

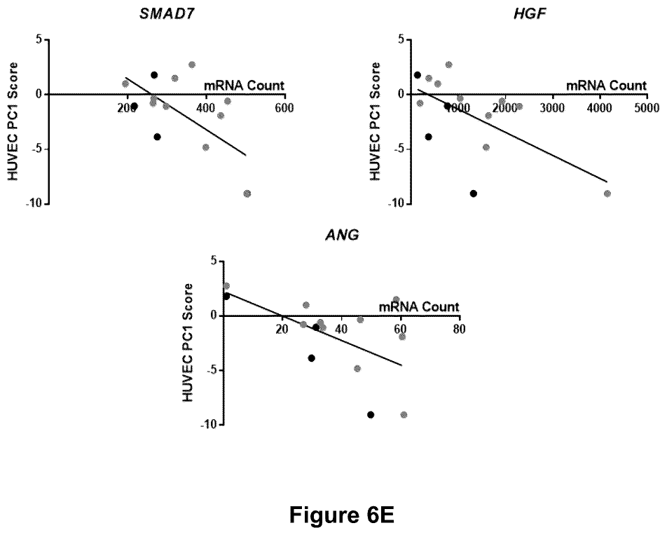

The full set of gene and soluble factor expression profiles as well as morphological features measured for MSC(AT) were examined for correlations to composite functional (PC1) scores (indicative of inflammation-resolving Mϕ polarization) generated for MSC(AT) immunomodulatory fitness. Under licensed conditions, MSC(AT) expression of THBS1 (R2=0.5481, p=0.0025, angiogenic gene), CCN2 (R2=0.3020, p=0.0418, encoding for the multifunctional growth factor), and EDN1 (R2=0.2854, p=0.0491, angiogenic gene) genes demonstrated significant inverse correlations with Mϕ PC1 composite scores (FIG. 6A, Table 3), suggesting that lower expression of these genes correlated with improved immunomodulatory MSC(AT) functionality. Correlations of Mϕ PC1 composite scores with ACTA2, PDCD1LG2, TNFAIP6, ANGPT1, and CXCL8 were near-significant. Under unlicensed conditions, MSC(AT) expression of six genes (CCN2, TSG101, THBS1, PDGFA, VEGFA, and EDIL3) were significantly inversely correlated with MϕPC1 composite scores, with CCN2 (R2=0.4934, p=0.0035), TSG101 (R2=0.4229, p=0.0087, negative growth regulator and regulator of vesicular trafficking), THBS1 (R2=0.3853, p=0.0135), and PDGFA (R2=0.3754, p=0.0152) displaying the strongest correlations (FIG. 6B, Table 3). Correlations of Mϕ PC1 composite scores with ACTA2, TGF-β protein, ANGPT1, and ANG were near-significant. Interestingly, many of the statistically significant and near-significant correlations were inverse correlations and they predominantly consisted of angiogenic-associated genes (THBS1, CCN2, EDN1, PDGFA, VEGFA, EDIL3, ANGPT1, and ANG).

Analysis of morphological features of MSC(AT) single cell suspensions derived under varying CPP conditions and/or donors revealed that cell diameter was significantly inversely correlated with inflammation-resolving Mϕ PC1 composite scores (FIG. 6C, Table 3), suggesting that smaller cells exhibit greater immunomodulatory properties, concordant with observations by Klinker et al. [15]. Correlation of Mϕ PC1 composite scores with cell circularity was near-significant (Table 3). Taken together, the significant and near-significant correlations between MSC(AT) gene expression and morphometric features with Mϕ pro-resolving polarization constituted a matrix of multivariate readouts that were considered as putative CQAs for informing MSC(AT) immunomodulatory fitness.

Generating a Matrix of Putative MSC(AT) CQAs Based on Correlation of Select Genes, Soluble Factors, and Morphological Features with Functional Angiogenesis

Linear regression analyses were also performed to investigate correlations of MSC(AT) markers with functional angiogenic HUVEC PC1 composite scores. A statistically significant positive correlation was found between HUVEC PC1 composite scores and levels of the pro-angiogenic factor VEGF in MSC(AT) conditioned medium measured under licensed conditions (R2=0.3048, p=0.0328) (FIG. 6D), while VEGF levels measured under unlicensed conditions showed a positive near-significant correlation (R2=0.2416, p=0.0743) (Table 4). No significant or near-significant correlations to HUVEC PC1 scores were observed for the other measured soluble factors. Regression analyses of HUVEC PC1 scores with MSC(AT) genes revealed significant inverse correlations for SMAD7 (R2=0.4237, p=0.0117, inhibitor of TGF-ß signaling), HGF (R2=0.3807, p=0.0187), and ANG (R2=0.3404, p=0.0285, pro-angiogenic marker) measured under licensed conditions (FIG. 6E). Correlations of HUVEC PC1 composite scores with expression of IDO1, TSG101, and CXCL8 were near-significant (Table 4). Under unlicensed conditions, expression of the chondrogenic marker SOX9 (R2=0.2906, p=0.0381) was significantly inversely correlated with HUVEC PC1 scores (Table 4). Inverse correlations of HUVEC PC1 composite scores with expression of TNFAIP6 were near-significant. No significant or near-significant correlations were observed between MSC(AT) cell diameter and circularity measurements with HUVEC PC1 composite scores. As above, the significant and near-significant correlations between expression levels of VEGF protein and the identified genes with HUVEC tube formation were considered as a matrix of multivariate readouts that served as putative CQAs for informing MSC(AT) angiogenic fitness.

### Statistical Rankings of the Matrix of Putative CQAs Ranks Different CPPs and Donors for Optimal MSC(AT) Immunomodulation and Angiogenic Fitness

Desirability profiling was applied as an analytical tool using the matrix of putative CQAs to empirically rank CPP conditions and MSC(AT) donors that favour immunomodulation or angiogenic fitness. Putative CQAs were selected using a broader p value threshold of p<0.1 (near-significance) based on the regression analyses presented above. This p value threshold was selected to filter the large initial panels of genes and soluble factors (curated based on literature) through a pipeline with a still flexible threshold that allowed selection of a relatively broad array of putative CQAs for MSC(AT) immunomodulatory or angiogenic basal fitness. The genes and soluble factors (measured under either licensed or unlicensed conditions), as well as morphological features were assigned minimization or maximization functions in the desirability analysis based on whether the correlation to functional outcomes was positive or negative (Tables 3 and 4). For example, markers with negative correlations to inflammation-resolving Mϕ or pro-angiogenic HUVEC PC1 composite scores (i.e., lower expression of the marker correlated to greater immunomodulatory/angiogenic function) were assigned minimization functions so that lower expression of the marker corresponded to a higher immunomodulatory or angiogenic desirability score. Furthermore, the R2 values were used as indicators of the amount of variation in the data explained by the model for each gene/soluble factor/morphological feature in correlation with either immunomodulatory or angiogenic functional outcomes. Thus, these values were applied as individual weightings for each gene, soluble factor, or morphological feature. For example, expression of THBS1 by licensed MSC(AT) correlated most strongly with inflammation-resolving Mϕ PC1 composite scores (R2=0.5481); the R2 value was used as a relative weighting such that THBS1 expression levels contributed more strongly to the overall desirability score relative to the other genes, soluble factors, and morphological features included in the analysis. Given that putative CQAs would require limits or ranges in order to be practically used, a range of data outputs were provided for each MSC(AT) readout (gene, soluble factor, and morphological feature) that correlated with immunomodulatory pro-resolving Mϕ polarization or HUVEC tube formation (Tables 3 and 4). These values inform the upper and lower limits of the putative CQA matrix readouts corresponding to MSC(AT) immunomodulation (Table 3) or angiogenic fitness (Table 4).

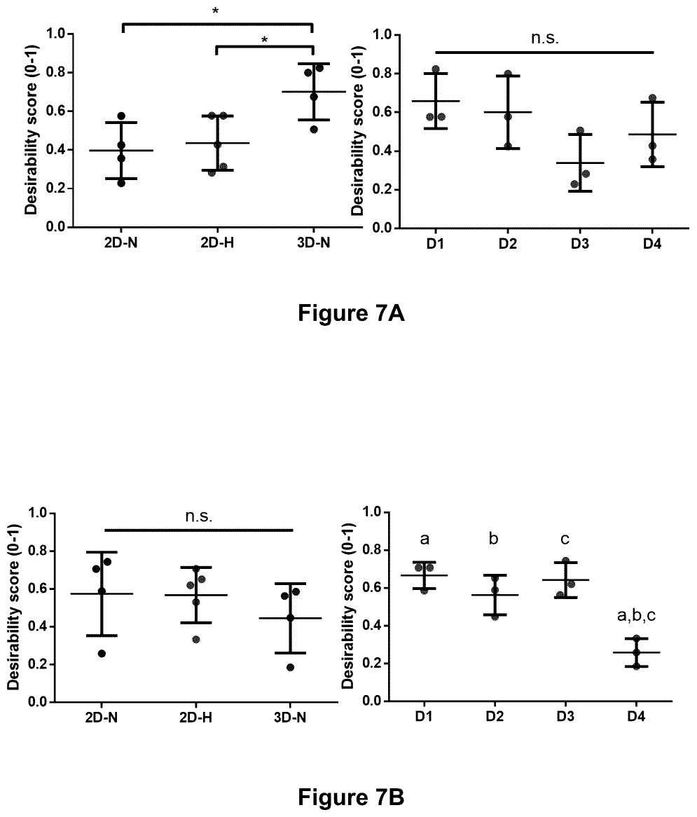

Using the panels of MSC(AT) genes, soluble factors, and morphological features that correlated with inflammation-resolving Mϕ PC1 scores (Licensed panel: ACTA2, ANGPT1, CCN2, CXCL8, EDN1, PDCD1LG2, THBS1, TNFAIP6; Unlicensed panel: ACTA2, ANG, ANGPT1, CCN2, EDIL3, PDGFA, TGF-β, THBS1, TSG101, VEGFA; Morphological features: cell diameter, cell circularity), desirability analysis revealed that MSC(AT) cultured under 3D-N configurations had significantly higher overall immunomodulatory desirability scores relative to 2D-H and 2D-N configurations (FIG. 7A), further corroborating that 3D-N MSC(AT) displayed augmented immunomodulatory properties. No significant differences in overall immunomodulatory desirability scores could be detected between donors.

Similar desirability analyses were performed using the angiogenic markers that correlated with angiogenic HUVEC PC1 scores (Licensed panel: SMAD7, HGF, ANG, IDO1, TSG101, CXCL8, VEGF protein; Unlicensed panel: SOX9, TNFAIP6, VEGF protein). The analysis revealed that donor differences dominated over the desirability rankings with Donors 1, 2, and 3 displaying significantly higher scores relative to Donor 4 (FIG. 7B), further suggesting that the effect of donor predominated over variations in CPP conditions when considering MSC(AT) angiogenic functionality. Donor 5 was excluded from the desirability analysis due to an incomplete dataset. No significant differences were observed for overall angiogenic desirability scores across variations in CPP conditions.

Altogether, using the matrix of putative CQAs that included MSC(AT) genes and soluble factors, morphological features, and immunomodulatory/angiogenic functional readouts, desirability analysis allowed empirically ranking of MSC(AT) immunomodulatory and angiogenic fitness across varying CPP conditions that enhance immunomodulation and angiogenic potency, and across MSC(AT) donors. The results showed that MSC(AT) cultured under 3D-N conditions displayed the highest overall immunomodulatory ranking, while specific adipose tissue donors had highest angiogenic desirability rankings.

### Discussion

In the present example, a matrix of putative quantitative CQAs were selected with a range of minimum and maximum values that define MSC(AT) immunomodulatory and angiogenic basal fitness in vitro and are sensitive enough to detect variations in CPPs and donor heterogeneity. Putative CQAs were generated based on statistically significant or near-significant correlations between MSC(AT) genes, soluble factors, and morphometric features and functional anchor in vitro readouts for immunomodulation (polarization of Mϕs to pro-resolving subtypes) and angiogenic potency (network of tube formation with HUVECs). Importantly, the putative CQAs can empirically rank the relative immunomodulatory or angiogenic fitness of MSC(AT) across varying CPP conditions and donors to identify: i) optimal cell culture conditions, or ii) optimal MSC(AT) donors with desired functionality. This approach is substantially more rigorous than use of surface identity markers which are frequently used as potential final product release criteria for MSCs therapeutics, despite clarifications made by the ISCT and the FDA [55,56] and given that these surface markers are not sensitive to variations in donor or culture conditions [57]. This approach is also aligned with recommendations from the ISCT to characterize MSC fitness using a matrix of assay readouts [58,59].

The CPP conditions investigated had a pronounced effect on immunomodulatory fitness as measured by in vitro MSC(AT)-mediated Mϕ polarization, with transient 3D-N conditions best augmenting MSC(AT) immunomodulatory basal functionality. Strong upregulated expression of immunomodulatory genes (TNFAIP6, ICAM1, and PRG4) and soluble factors (HGF, TGF-β, IL-1RA) by MSC(AT) cultured under 3D-N conditions was shown. The data also corroborates previous work that showed 3D MSC(M) spheroids promoted an inflammation-resolving macrophage phenotype in vitro and suppressed inflammation in a mouse model of peritonitis [31,60]. In contrast to the work using xeno-free 3D culture conditions, others have shown that 3D MSC(M) spheroids lose their ability to suppress pro-inflammatory macrophage activities in vitro when cultured using xeno-free medium [36]. These differences may be attributed to different methods used for generating 3D cell aggregates. The combination of 3D and hypoxic culture was also investigated and no additive effects were found using a targeted panel of both immunomodulatory and angiogenic genes. Based on this data, 3D-N and 2D-H conditions were evaluated separately recognizing that these are currently being explored as CPPs that enhance MSC(AT) fitness. Nevertheless, the present methodological approach provides a platform for other investigators looking to evaluate and optimize different CPP conditions individually or in combinations using the provided range of quantitative CQAs.

MSC(AT) cultured under 2D-H conditions showed similar gene/soluble factor expression profiles relative to 2D-N conditions, with upregulation of only a few select immunomodulatory markers (PTGS2 and PD-L2), and concordantly exhibited intermediate immunomodulatory functions. There is limited data on the immunomodulatory functions of MSCs cultured under hypoxic conditions, but previous work has shown that hypoxic culture can augment T cell inhibition mediated by rat MSC(M) [30] and human MSC(AT) [29]. While varying CPP conditions exerted a greater effect on in vitro immunomodulation, increased expression of some pro-inflammatory Mϕ markers was observed in co-cultures with MSC(AT) and this was partly donor-driven. Previous work has shown that MSCs can induce a mixed Mϕ phenotype which may be important for augmenting Mϕ microbicidal functions [61], and the present work suggests an effect of donor heterogeneity on inducing these mixed Mϕ phenotypes.

In contrast to results measuring immunomodulatory fitness, effects of donor heterogeneity predominated over effects of variations in CPPs in modulating angiogenic basal functionality of MSC(AT). This result is surprising given previous work that has shown augmented angiogenic functions of MSC(AT) cultured in 3D aggregates [32,34], and extensive literature showing augmented angiogenic functions of MSC(AT) cultured under hypoxic conditions [27,28,35]. Culture under 2D-H conditions augmented pro-angiogenic functions of MSC(AT) only for select donors. This discrepancy with prior literature could be related to the transient (16-20 h) incubations used for 3D/hypoxic priming. Furthermore, differences in the 3D culture method used in this study (e.g., culture using xeno-free medium), and a relatively high level of hypoxic oxygen tension (38 mmHg or approximately 5% 02) could account for differences relative to previous work. Nonetheless, the data demonstrated a significant effect of donor heterogeneity and suggests that this predominates over variations in CPPs in dictating MSC(AT) basal angiogenic functionality.

The selection of in vitro functional MSC(AT) readouts were carefully chosen to anchor the matrix of multivariate read-outs and refine putative CQAs according to two therapeutically relevant properties of MSCs: immunomodulation and angiogenesis. In terms of immunomodulatory functions, MSC(AT)-mediated Mϕ polarization toward pro-resolving subtypes was evaluated, recognizing that Mϕs represent primary effector cells for mediating MSC therapeutic functions in multiple diseases, including GVHD [9], colitis [62], and osteoarthritis [8]. While effects of MSCs on T cells are more frequently employed as an in vitro readout to evaluate MSC immunomodulatory potency, this is not a gold standard as previous work has also demonstrated lack of correlation to clinical efficacy of MSC(M) in GVHD patients [10]. Angiogenic functionality of MSC(AT), was measured using the widely-accepted HUVEC tube formation read-out, which recapitulates several aspects of in vivo angiogenesis including endothelial cell adhesion, migration, alignment, and formation of tubules [54]. This assay has also been used to evaluate angiogenic functionality for clinical-grade MSCs [19,38].

The functional in vitro read-outs were used as anchors in the putative CQA matrix allowing refinement of a putative set of 60 CQAs (48 genes of interest, 10 soluble factors, and 2 morphometric features) down to 20 CQAs that correlated to in vitro Mϕ polarization, and 10 putative MSC(AT) CQAs that correlated to in vitro HUVEC tube formation. Interestingly, angiogenic genes measured under both licensed and unlicensed conditions negatively correlated with Mϕ polarization toward inflammation-resolving subtypes. Conversely, increased expression of select immunomodulatory genes were also negatively correlated to greater in vitro HUVEC tube formation. This data suggests an inverse interplay between MSC(AT) immunomodulatory and angiogenic fitness and corroborates previous work by Boregowda et al. [17,63]. The present analysis supported the utility of previously reported MSC characteristics that correlate to immunomodulatory fitness—including TNFAIP6 expression (encoding for TSG6) [8,64,65], TGFB1 expression [8], and cell diameter [15]); and to angiogenic fitness, including increased VEGF expression [38]. Furthermore, the results suggest that different CPP conditions or MSC(AT) donors should be selected/optimized for clinical applications depending on the target disease indication and desired therapeutic mechanism.

Desirability analysis was applied to empirically rank the immunomodulatory or angiogenic basal fitness of each MSC(AT) donor or CPP condition. The results mirrored the functional assay outcomes, demonstrating a greater effect of CPPs on MSC(AT) immunomodulatory functions, and a greater effect of donor heterogeneity on angiogenic functions. Notably, MSC(AT) characteristics (genes, soluble factors, and morphological features) were selected for inclusion in the desirability analysis based on the strength of their correlations to the functional anchor assays, and the R2 goodness-of-fit values were used as a relative “importance ranking” for each of the MSC(AT) characteristics. Thus, MSC(AT) characteristics with significant correlations to functional assay outcomes (lower p value and higher R2 value) contributed to the overall desirability score to a larger extent compared to characteristics with near-significant correlations (higher p value and lower R2 value). Based on these analytical methods, it is expected that the overall desirability rankings would closely match the results from the original functional assay outcomes as they were used to determine the relative strength of contributions to the overall desirability score calculations. Nonetheless, this empirical approach has utility in future studies where similar desirability analysis can be used as an unbiased tool to rank the immunomodulatory or angiogenic fitness of a given population of MSCs based on combinatorial analysis of a matrix of curated genes, soluble factors, and morphological features only. The same relative importance rankings for the set of putative CQAs could be applied, circumventing the need to conduct lengthy, non-high-throughput functional analyses. To the present inventors' knowledge, this is the first reported application of desirability analysis for understanding MSC potency attributes.

In the present example, in vitro functional assays were used as surrogate read-outs for MSC(AT) immunomodulatory and angiogenic fitness. These analyses generated relatively broad panels of putative CQAs selected based on correlation analyses to in vitro functional readouts with a p value threshold set to p<0.1. The CQAs that were evaluated were, necessarily, termed here as “putative”; ultimately these putative CQAs can be narrowed down further and validated in clinical studies where specific MSC(AT) CQAs may be more or less relevant depending on the target disease and/or disease stage. To accelerate clinical translation of MSC products, there is a need for simple, robust, and reproducible potency readouts, which the panel of quantitative and correlative CQAs described herein supply. Importantly, two in vitro functional readouts were used as surrogate anchors for clinical efficacy. Future studies may substitute disease-specific biomarkers as recommended by Krampera and Le Blanc [3] in lieu of the in vitro functional readouts; correlation of a refined list of MSC CQAs with changes in disease attributes would be the ultimate validation. In this vein, creation of registry databases that would allow logging of large datasets, including the proposed CQA matrix used to characterize MSC(AT), may be practical to allow different sponsors to query candidate CQAs in their respective clinical MSC products and examine correlations with disease-specific biomarker changes and thus therapeutic efficacy.

Variable clinical responses to MSC treatments can arise from several factors, including through heterogeneity in donor or CPP factors explored here, but also through in vivo interactions with host tissues [3,5]. While host responses were unable to be captured using the in vitro assays applied in this work, defining basal thresholds of MSC immunomodulatory and/or angiogenic fitness with defined ranges of quantitative CQAs enables a greater likelihood of eliciting stronger therapeutic effects, as hypothesized by the ISCT MSC Committee [66]. MSC basal fitness levels are especially relevant to therapeutic potency for extravascular delivery where in vivo persistence allows for release of paracrine factors [66], compared to vascular delivery where efficacy may rely more on proclivity of MSCs to be rapidly cleared by host immune cells as evidenced in GVHD by Galleu et al. [9]. Another limitation of the present study design was the sourcing of MSC(AT) from patients with knee or hand osteoarthritis which was based on tissue availability. It should be noted that these MSCs satisfied minimal surface marker expression criteria for MSC(AT) [42], and the adipose tissue depots were located external to the affected joints from patients with mild to moderate osteoarthritis (KL grade 0-2). However, both local joint factors and systemic factors can contribute to the pathology of osteoarthritis [67], and thus potential effects on the isolated MSC(AT) cannot be discounted.

Taken together, the present example provides a systematic, empirical approach to evaluate the effects of variations in CPPs, specifically those that can non-genetically enhance MSC immunomodulatory and/or angiogenic properties, and donor heterogeneity on MSC(AT) critical attributes. A matrix of putative, quantitative CQAs was established with a range of minimum and maximum values based on correlations of multivariate readouts of MSC(AT) cell morphology, gene expression, and soluble factor expression with functional readouts that served as an anchor in the analysis. These functional assays are relevant in determining CQAs for MSC(AT). Importantly, the empirical approach can be adapted and applied to future clinical studies where changes to disease-specific biomarkers may be substituted in lieu of in vitro functional readouts to serve as the anchor. The putative CQA matrix empirically ranked the effects of CPPs or donor heterogeneity on desired immunomodulatory or angiogenic MSC(AT) basal fitness, and showed differential sensitivity to these variables, suggesting that a one-size-fits-all approach is not suitable for manufacturing MSC(AT). Ultimately, the present analysis identified putative CQAs that may be used to prospectively screen potent MSC(AT) donors and select specific CPP conditions to enhance for desired MSC basal fitness ranges.

### Example 2

This Example provides additional data and analyses that validate the panel of critical quality attributes (CQAs) identified in Example 1 for measuring the basal immunomodulatory fitness of mesenchymal stromal cells (MSCs).

A subset of the previously identified mRNA transcripts (part of the putative CQAs) were measured in bone marrow-derived MSCs (MSC(M)) under pro-inflammatory licensing conditions. Bone marrow represents the most common tissue source for deriving MSCs used in clinical trials [71]. Retrospective correlative analyses that used clinical patient-reported outcome measures (PROM) data were applied to further demonstrate the validity of these CQAs for measuring basal immunomodulatory fitness of MSCs. The MSC(M) were obtained from biobanked samples derived from nine different knee osteoarthritis (OA) patients treated with a single intra-articular injection of autologous MSC(M) in the inventors' previous nonrandomized open-label dose-escalation phase I/IIa clinical trial [8]. MSC-mediated immunomodulation may represent a primary mechanism of action for MSC therapy in knee OA [72]. Furthermore, basal immunomodulatory fitness of MSCs correlates to patient-reported outcome measures (PROMs) in knee OA patients treated with autologous MSCs [8,70].

The algorithm was further refined based on desirability analysis that enabled empirical ranking of individual MSC(M) donors in terms of their immunomodulatory fitness and clinical efficacy data in knee OA.

### Methods

### Study Design and Patients

Patients were recruited to this nonrandomized, open-label, dose-escalation phase I/II clinical trial with informed consent as reported by the inventors previously [8] (ClinicalTrials.gov Identifier: NCT02351011). Ethics approval was granted by the UHN Research and Ethics Board (REB 14-7909) and Health Canada. Eligibility criteria included: i) radiographic and symptomatic knee OA, Kellgren-Lawrence (KL) grade III-IV; ii) Age: 40-65 years; Body Mass Index (BMI)<30; iii) failed conservative management for a minimum of 6 months; iv) no history of intra-articular hyaluronic acid, cortisone, or platelet-rich plasma within the previous six months; v) clinically stable knee; and, vi) relatively neutral knee alignment (<5 degrees, measured by 4-foot standing antero-posterior radiographs), as well as other inclusion/exclusion criteria detailed by the inventors previously [8]. Patients received a single intra-articular autologous MSC(M) injection at doses of 1×106, 10×106, and 50×106 cells (N=4 patients/dose group). Clinical follow-up was performed over 24 months after MSC(M) injection. The Knee Injury and Osteoarthritis Outcome Score (KOOS) was applied as a patient-reported outcome measurement tool and the scale was inverted such that higher scores indicate worse outcomes. Both delta and percentage change KOOS values were calculated using scores collected at follow-up time points relative to baseline in order to capture absolute changes, as well as changes that account for baseline values, respectively. While delta values correspond to the magnitude of difference relative to baseline, they do not account for differences at baseline; percentage change values do control for baseline scores but can be inflated when baseline scores are low. Both are considered relevant measures of change according to international criteria for evaluating OA therapeutics and are therefore included throughout this study. Delta and percentage change values were calculated such that positive values indicated improvement.

### MSC(M) Isolation and Culture

Biobanked MSC(M), retained by consent of OA patients under UHN REB 16-5493, were thawed and expanded for additional CQA analysis, following the same expansion protocol that was used in the clinical trial [8]. For all experiments, MSC(M) were used between passage 4 and 5.

### MSC(M) Gene Expression Analysis

MSC(M) were seeded on 24-well plates at 30,000 cells/well in proliferation medium and licensed with pro-inflammatory cytokines (IFNγ, 30 ng/ml; TNFα, 10 ng/ml; and IL-1β, 5 ng/ml), as described in Example 1. After 24 hours of culture, Trizol-chloroform extraction was performed to isolate RNA, and cDNA was generated using SuperScript™ IV VILO™ Master Mix (Invitrogen, Waltham, USA). FastStart Universal SYBR Green Master Mix (Roche, Basel, Switzerland) and custom primers (Table 14) were used for qPCR on a QuantStudio™ 5 system (ThermoFisher). Results were normalized (delta Ct, ΔCt) using reference genes (GAPDH, GUSB) and presented as negative ΔCt such that higher (more positive values) indicate a higher level of expression.

### Data Analysis and Statistics

GraphPad Prism 6.0 (La Jolla, USA) and JMP Pro 17 (Cary, USA) software were used for statistical analyses and to create plots. Statistical tests are specified in the figure and table captions. Spearman's correlation was selected as a non-parametric statistical test to investigate associations between KOOS changes (delta or percentage change) and other variables, given that variables within the dataset violated assumptions of normality and homoscedasticity. Spearman's correlation offers further advantages including that it does not assume linear associations (instead, it assesses monotonic relationships), and that dependent/independent variables do not need to be specified. P values derived from Spearman's correlation were not corrected for multiple comparisons given that common methods for multiple comparison corrections rely on assumptions of normality. P values less than 0.05 were considered statistically significant.

Desirability analysis, as a tool for multiple response optimization developed by Derringer and Suich [47] was performed in JMP Pro 17. The analysis assigns individual desirability scores between 0 and 1 based on the range of data values for each gene, with 1 representing a highly desirable response and 0 representing an undesirable response. Minimization or maximization functions were assigned to each gene based on whether upregulation or downregulation of the gene was considered desirable, using the weighting assignments described in Example 1 or results from new correlation analyses to justify the selection of CQAs used (more details described in Results). For analyses incorporating results from new correlation analyses with PROMs, the highest absolute r_{S} value obtained for each gene was used as weighting, such that genes with higher r_{S} values contributed more strongly to the overall desirability score relative to genes with lower r_{S} values. Overall desirability scores were calculated using the geometric mean of individual desirability scores corresponding to each gene, and thus also range from 0 (undesirable) to 1 (highly desirable).

For a subset of analyses, OA patients were classified as Function-Pain Responders, Function Responders, Pain Responders and Non-Responders in order to compare desirability scores for MSC(M) derived from patients under this classification. The OMERACT-OARSI criteria offers a rigorous classification system developed using data from large, powered placebo-controlled drug trials for hip and knee OA [69]. These criteria categorize patients as responders according to the following: i) improvement in pain or in function≥50% and absolute change≥ 20; OR, ii) improvement (≥20% and absolute change≥ 10) in at least two of the following: a) pain, b) function, c) patient global assessment (not measured in our trial). Given the limited sample size and lack of patient global assessment metric in the inventors' previous clinical trial, patients were categorized based on changes in KOOS function in daily living (ADL; corresponding to a function score) and KOOS Pain only, and thresholds selected based on the OMERACT-OARSI criteria for function and pain scores of delta value≥ 10 AND percentage change≥ 20% to determine responder status were applied [69]. Thus, the criteria applied for determining responders was more relaxed to accommodate for the smaller sample size and lack of patient global assessment scores, and it consisted of the following: i) Function-Pain Responders (patients exceeding thresholds in both ADL and Pain scores), ii) Function Responders (patients exceeding thresholds in ADL scores), and iii) Pain Responders (patients exceeding thresholds in Pain scores).

### Results

Previously Identified Immunomodulatory Critical Quality Attributes as Identified in MSC(AT) Correlate to Patient Responder Status when Evaluated in MSC(M)

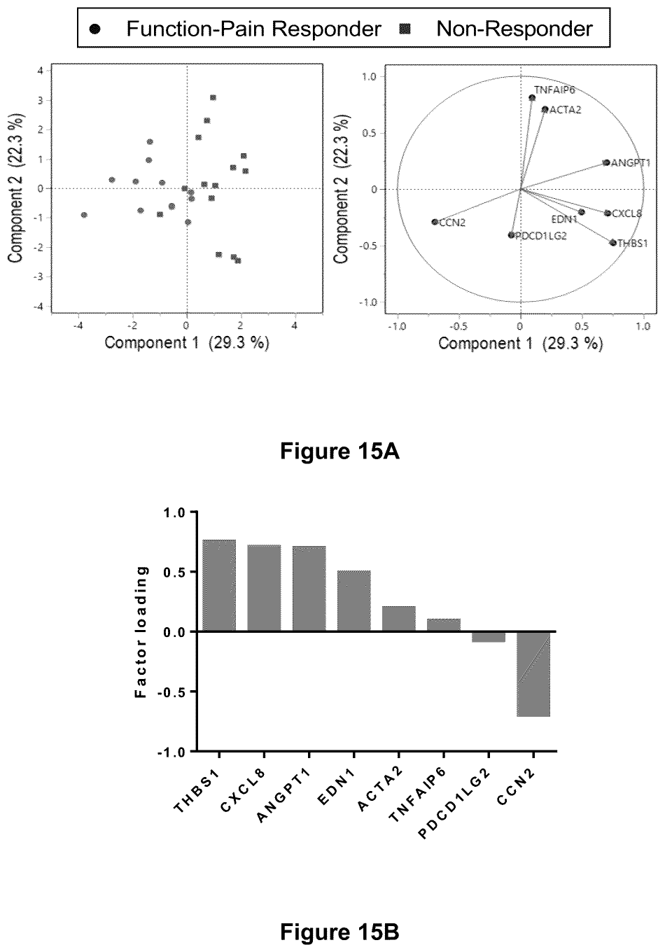

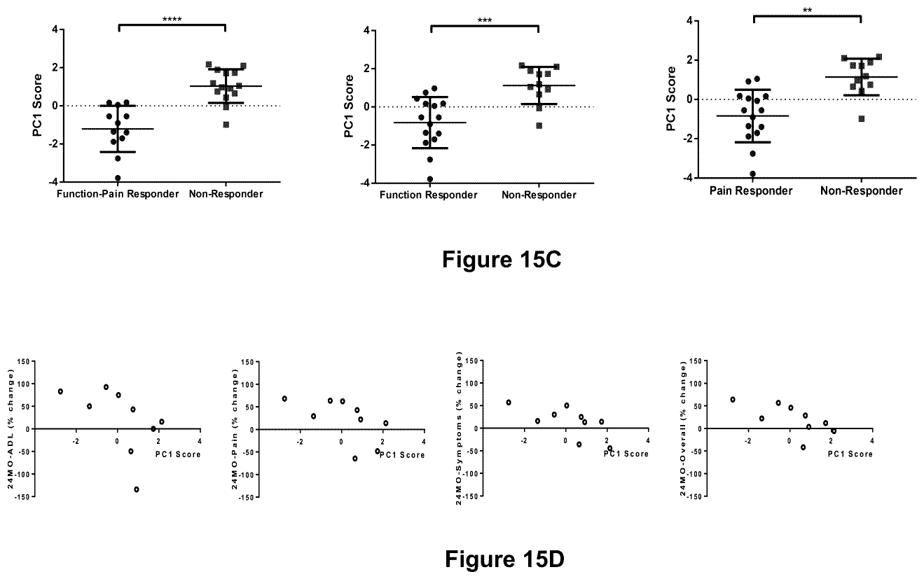

Clinical evidence supportive of immunomodulatory mechanism of action of MSC(M) injection in knee OA has been studied [8]. Given that autologous MSC(M) were evaluated in a clinical trial and that donor heterogeneity is a major variable influencing the basal fitness of MSC(M) [22], the inventors probed the basal immunomodulatory fitness of MSC(M) derived from these patients. A curated panel of mRNA transcripts expressed by MSC(M) under pro-inflammatory licensing conditions were measured that were previously identified as putative CQAs for MSC(AT) immunomodulatory fitness in Example 1. Principal Component (PC) analysis was applied as an unsupervised dimension reduction tool that revealed separation between MSC(M) samples independently classified as Function-Pain Responders compared to Non-Responders along the PC1 axis (accounting for 29.3% of total variance in the dataset), with higher expression of CCN2 and lower expression of angiogenic markers THBS1, CXCL8, and ANGPT1 contributing most strongly to higher PC1 scores (FIG. 15A, 15B). Individual PC1 scores plotted for each donor demonstrated significantly lower PC1 scores for Function-Pain Responders, Function Responders, and Pain Responders relative to Non-Responders (FIG. 15C), indicating that the expression profile of these mRNA transcripts distinguished MSC(M) donors independently classified as responders and non-responders, based on clinical outcomes. Notably, Example 1 identified this set of licensed genes measured in the current study, including CCN2, as being negatively correlated to MSC(AT) immunomodulatory basal fitness as measured through in vitro MO polarization read-outs. However, higher expression of CCN2 (encoding for the pleiotropic signaling protein, connective tissue growth factor, CTGF) was generally observed in responder MSC(M) samples, which may represent a tissue-of-origin difference between MSC(M) and MSC(AT), or a difference in MSC(M) basal fitness within the context of OA therapy.

Correlation analyses using individual transcripts demonstrated that higher basal gene expression of ANGPT1 in MSC(M) significantly negatively correlated to improvements (delta and/or percent change values) in KOOS Pain, ADL, function in Sport and Recreation (Sports/Rec), knee-related quality of life (QOL), and Overall KOOS at 12 months relative to baseline, as well as improvements in KOOS ADL at 24 months relative to baseline (Table 15). In contrast, higher levels of basal CCN2 gene expression in MSC(M) samples significantly positively correlated to improvements (delta and/or percent change values) in KOOS Symptom, Pain, QOL, and Overall KOOS at 12 months relative to baseline. Furthermore, PC1 scores were considered as a composite score for the basal immunomodulatory gene expression profile, and robust negative correlations were observed between PC1 scores and improvements (delta and/or percent change values) in KOOS Symptoms, ADL, and Overall KOOS at both the 12- and 24-month time points relative to baseline, as well as KOOS QOL (12-month time point only) and KOOS Pain (24-month time point only) (FIG. 15D, Table 15). Taken together, these findings strongly support the utility of these curated transcripts as immunomodulatory CQAs for MSC(M) in knee OA.

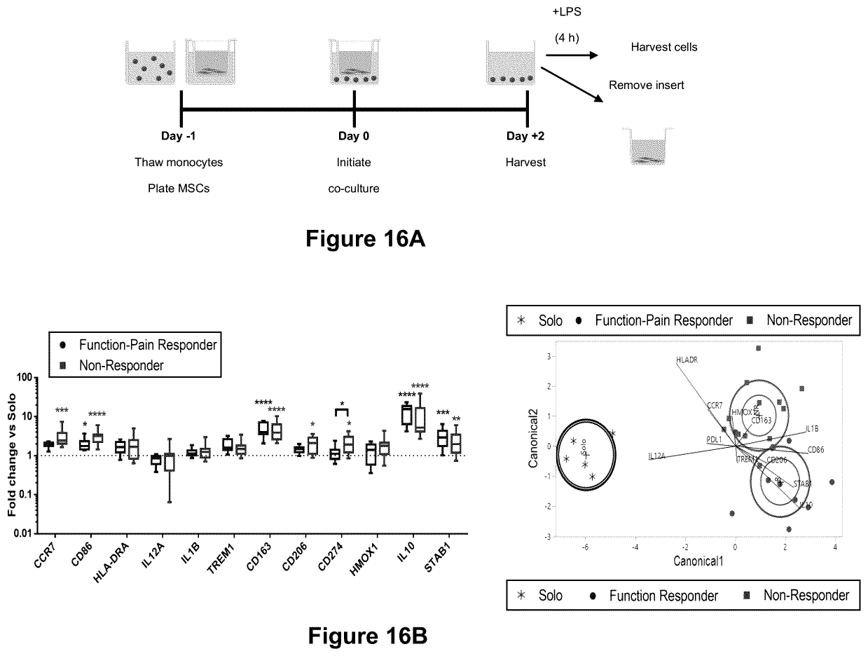

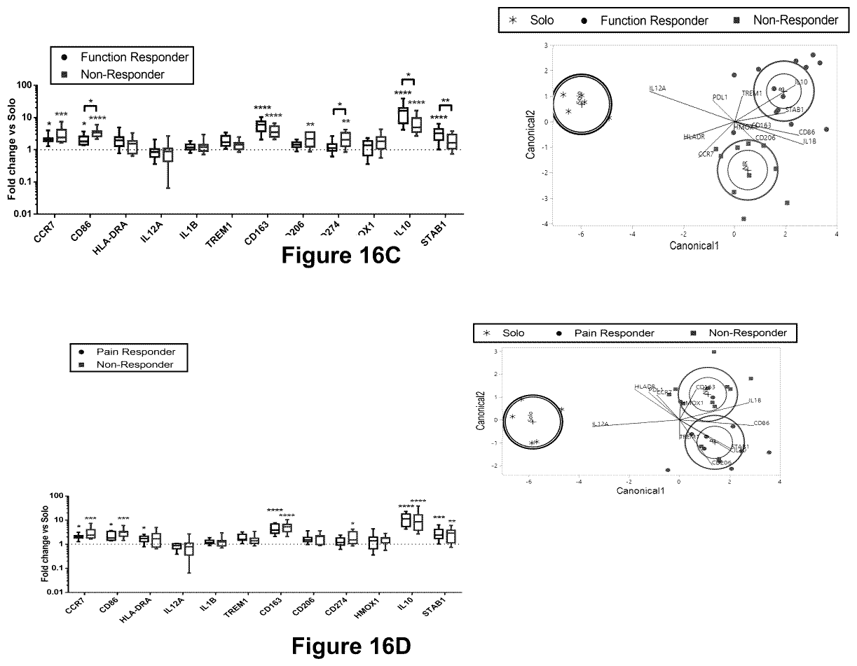

In addition to the assessment of putative immunomodulatory CQA transcripts, functional in vitro Mϕ polarization read-outs were employed using indirect co-culture of MSC(M) with human peripheral blood-derived Mϕ (FIG. 16A). After 48 hours of co-culture, expression of pro-inflammatory (CCR7, CD86, HLA-DRA, IL12A, IL1B, TREM1) and inflammation-resolving (CD163, CD206, CD274, HMOX1, IL10, STAB1) genes were measured as Mϕ phenotypic markers [43,44]. Discriminant analysis revealed that MSC(M) derived from Function-Pain Responders induced a distinct Mϕ gene expression profile relative to Non-Responders and Mϕ cultured without MSC(M) (FIG. 16B, Table 16). While Non-Responders induced significantly higher levels of inflammation-resolving markers CD274 and CD206, they also upregulated expression of CCR7 and CD86, suggestive of a mixed Mϕ phenotype.

Interestingly, discriminant analysis further demonstrated distinct clustering and greater separation of Mϕ phenotypic profiles when samples were analyzed based on co-culture with Function Responders versus Non-responder MSC(M) groups (FIG. 16C), and the separation was diminished when samples were classified according to Pain Responder categorization (FIG. 16D). Indeed, the discriminant model fit was improved when samples were classified by Function Responders criteria vs. either the Function-Pain Responder or the Pain Responder classification criterion indicated by fewer misclassified data points and an increased Entropy R2 value for the model (Table 16). Notably, a more robust inflammation-resolving Mϕ phenotype was observed in co-cultures with MSC(M) derived from Function Responders, with significantly upregulated expression of IL10 and STAB1 as well as reduced expression of CD86 in Mϕ co-cultured with Function Responders compared to Non-Responders (16C). An exception was observed for CD274 which was upregulated in Non-Responders compared to Function Responders. Overall, results from functional in vitro Mϕ polarization read-outs indicated that MSC(M) samples derived from Function Responder patients displayed more potent immunomodulatory basal fitness as indicated by in vitro Mϕ polarization toward inflammation-resolving phenotypes. This indicates that MSC(M) with more potent basal immunomodulatory fitness (based on Mϕ polarization) also induced greater functional improvements based on KOOS measurement.

### Desirability Analysis of Immunomodulatory CQA Genes in MSC(M) Using the Previous Analysis of MSC(AT) as a Training Dataset

In Example 1, desirability analysis was performed to empirically rank the basal immunomodulatory fitness of MSC(AT) prepared using various critical process parameters (CPPs) and derived from multiple MSC(AT) donors. Input parameters (including relative importance values as weightings and/or minimization/maximization functions) for the desirability analysis were assigned based on Pearson correlations of individual MSC(AT) characteristics with in vitro Mϕ polarization readouts as a surrogate measure for basal immunomodulatory fitness (see Example 1). In this new analysis using MSC(M) samples derived from nine different OA patient donors, this previous work on MSC(AT) was considered as a “training dataset” by applying the same input parameters in a new desirability analysis of the MSC(M) CQA gene expression profile. Analysis was performed using equal weighting of the CQA genes (i.e., using only the minimization/maximization functions specified in Example 1), as well as with the R2 correlation coefficient values used as weightings derived from the MSC(AT) training dataset.

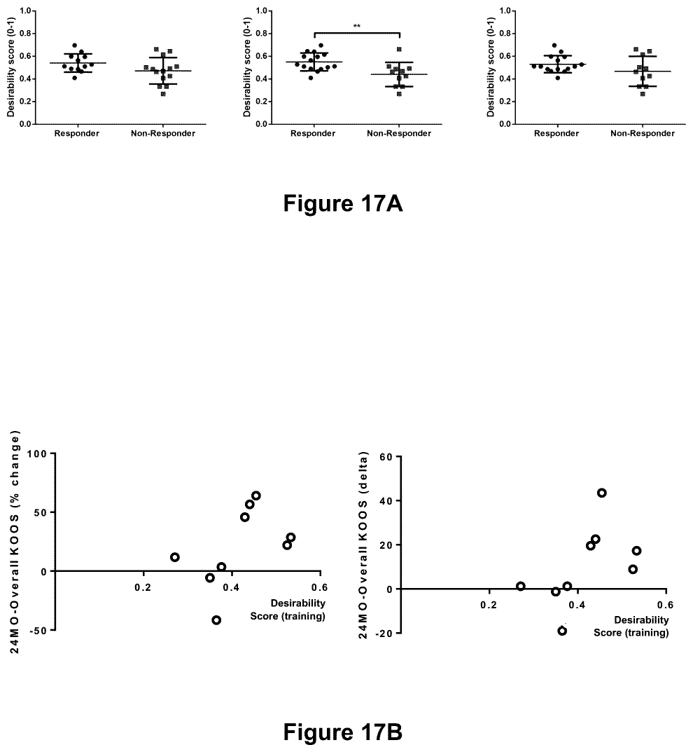

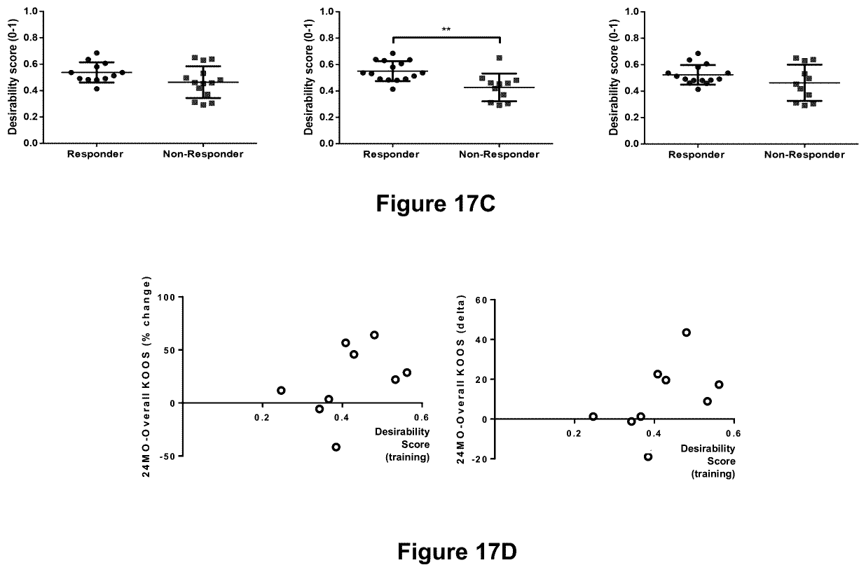

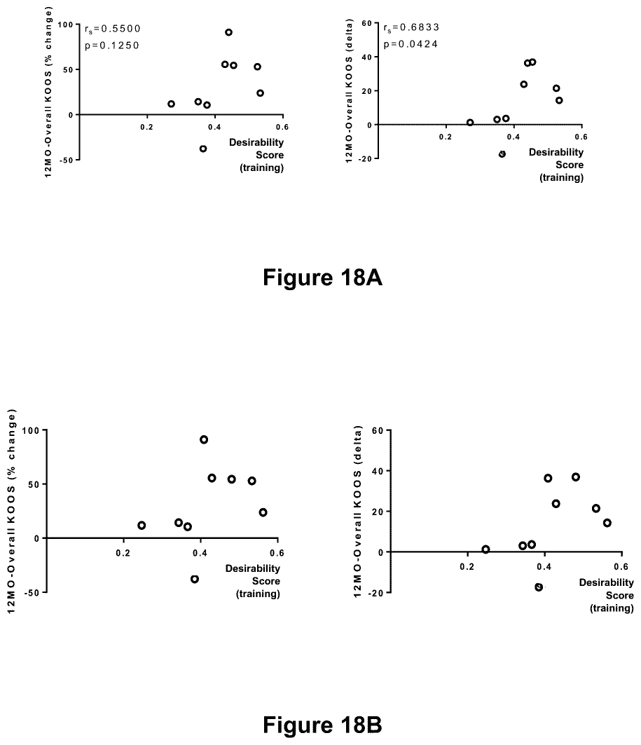

In the desirability analysis using equal weighting of the CQA genes, overall desirability scores trended higher for MSC(M) derived from donors classified as Function-Pain Responders and Pain Responders compared to Non-Responders, but the scores were statistically significantly higher only for MSC(M) classified as Function Responders compared to Non-Responders (FIG. 17A). The overall desirability scores were positively correlated to improvements (percentage change and delta values) in Overall KOOS at 12 and 24 months relative to baseline, but a statistically significant positive correlation was observed only for improvements (delta values) in Overall KOOS at 12 months relative to baseline (FIG. 17A, FIG. 18A).

When desirability analysis was performed using weighted input parameters (using previously obtained R2 values as relative importance values for each CQA gene based on data from the MSC(AT) training dataset), similar results were obtained, including significantly higher overall desirability scores for MSC(M) derived from donors classified as Function Responders vs. Non-Responders and non-significant positive correlations between overall desirability scores with improvements in Overall KOOS at 12 and 24 months relative to baseline (FIG. 17C, 17D, FIG. 18B).

These data support the use of desirability analysis to empirically rank the basal immunomodulatory fitness of individual MSC(M) donors. It also corroborates analyses the inventors [8] and others have shown to demonstrate augmented basal immunomodulatory fitness of MSC(M) donors that induce functional improvements in knee OA patients. While most of the correlations analyzed for MSC(M) desirability scores and changes in Overall KOOS, were not statistically significant, the correlations showed positive trends, indicating that higher immunomodulatory desirability scores of MSC(M) donors (based on previous panel of weighted CQAs) generally correlated to greater PROM improvements in knee OA patients treated with autologous MSC(M), and are therefore supportive of the use of the panel of putative CQAs and the algorithm for weighting them. Nonetheless, the data suggested that refinements could further improve the desirability analysis algorithm to improve sensitivity for individual MSC(M) donors, as explored below.

### Refined Desirability Analysis of Immunomodulatory CQA Genes in MSC(M) Allows Ranking of Clinically Efficacious MSC(M) Donors for Knee Osteoarthritis

In order to enable ranking of individual MSC(M) donors based on their clinical efficacy in knee OA, the desirability analysis algorithm was further refined. Results were considered for all Spearman's correlations between the individual CQA genes expressed by MSC(M) with improvements in KOOS after MSC(M) injection at 12 and 24 months relative to baseline (Table 15). Using this data, the highest absolute value for the Spearman's rho correlation coefficient corresponding to each individual MSC(M) gene was selected. This value is a measure of the fit of a monotonic function between the expression levels of the gene with improvements in KOOS subscales based on relative rank-orders. Table 17 provides a summary of the selected correlations with highest r_{S} values between MSC(M) CQA genes with improvements in KOOS subscales at 24 months relative to baseline. The directionality of the correlations was used to assign minimization or maximization functions to each CQA gene. Moreover, the Spearman's rho correlation coefficient was used to assign weightings (i.e., importance values) to each CQA gene. Therefore, given that p values and correlation coefficients are inter-related, markers with significant correlations (positive or negative) contributed to a greater extent to the overall desirability score relative to markers with lower p values and correlation coefficients.

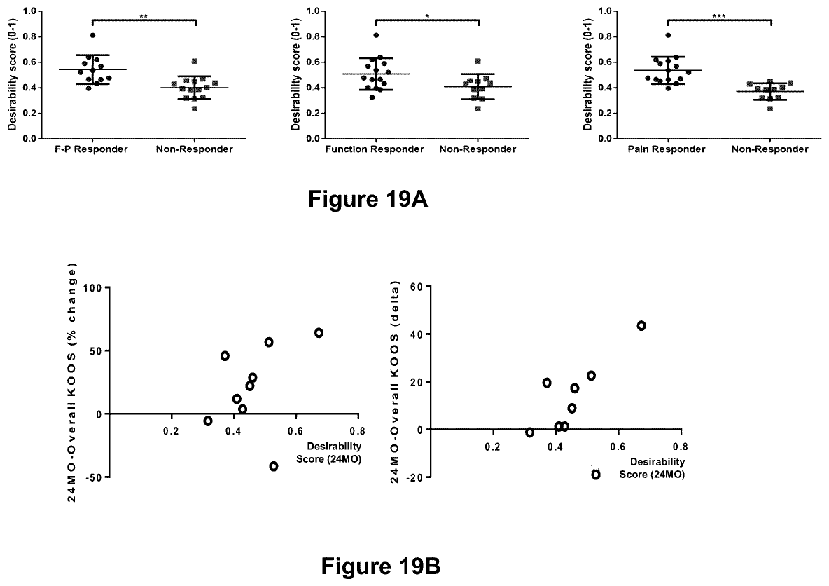

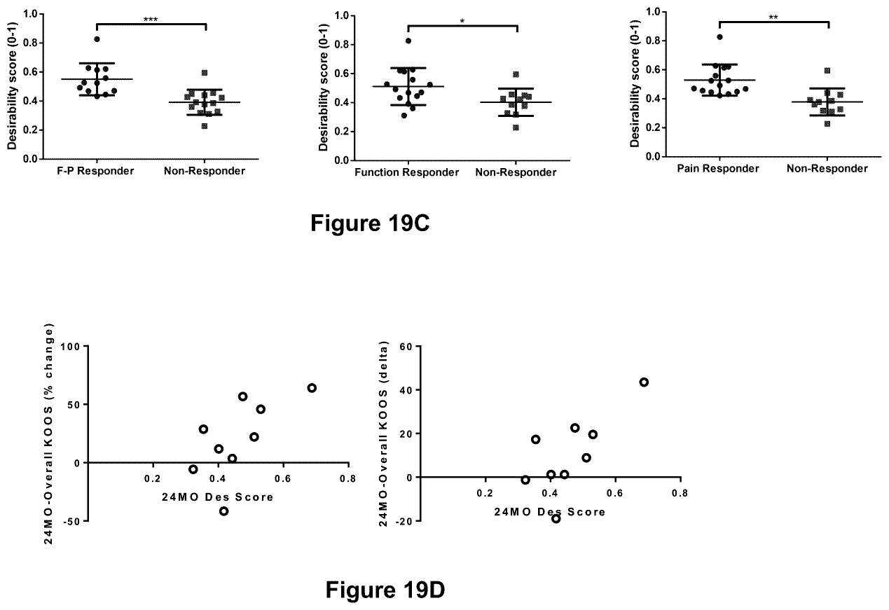

Using this approach, the desirability analysis was repeated and the overall desirability scores of MSC(M) derived from donors classified as Function-Pain Responders, Function Responders, and Pain Responders were all significantly higher relative to Non-Responders, and this was consistent whether genes were equally weighted (FIG. 19A) and weighted based on r_{S} values (FIG. 19C). The desirability scores also showed robust and significantly positive correlations with improvements in Overall KOOS at 24 months relative to baseline, using both the equal gene weighting (FIG. 19B) and the r_{S} value-weighted approaches (FIG. 19D).

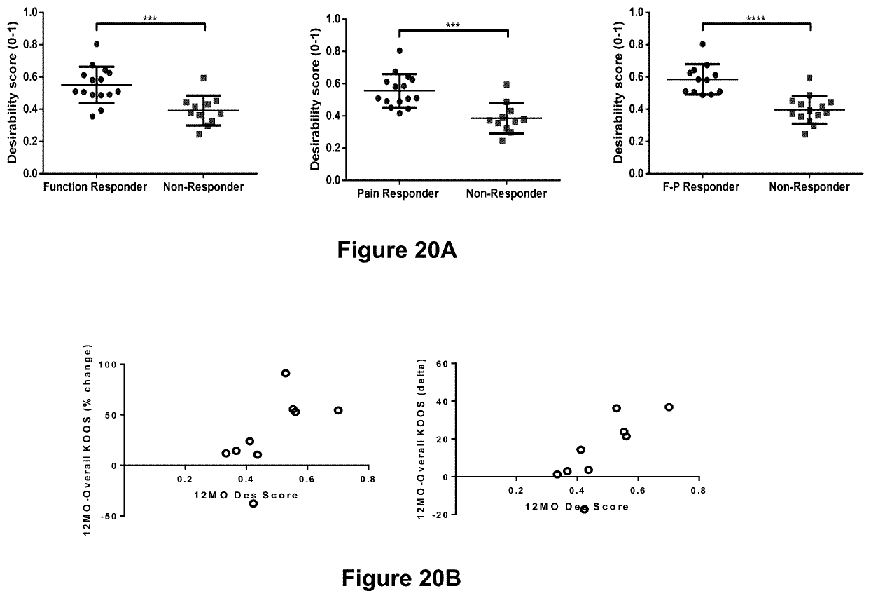

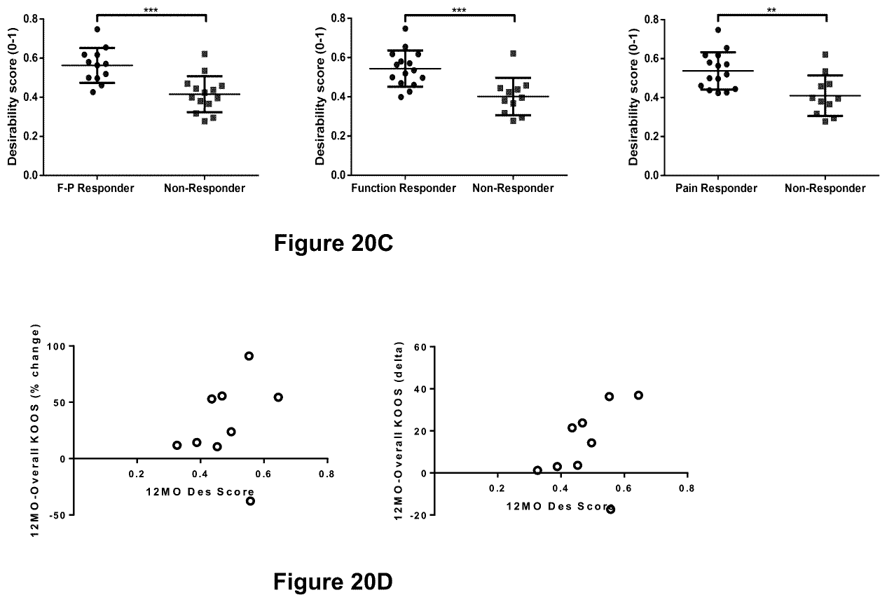

In addition to the above, desirability analysis was performed using results of correlations performed with 12-month follow-up PROM data, as this was considered the study endpoint in the inventors' previous work [8]. A summary of the highest r_{S} values obtained for each CQA gene is provided in Table 18. The results of the desirability analysis were similar, with significantly higher desirability scores observed for all categories of Responder patients relative to Non-Responders (FIG. 20A,20C). In desirability analysis using equal weighting of CQA genes, the desirability scores MSC(M) donors positively correlated to improvements in Overall KOOS at 12 months relative to baseline, but the correlation was statistically significant for improvements calculated based on delta values and not for percentage change values (FIG. 20B). In desirability analysis using r_{S} value weightings of CQA genes, the desirability scores for MSC(M) donors significantly positively correlated to improvements in Overall KOOS (both delta and percentage change values), indicating an improved fit (FIG. 20D). These data indicate the validity and reproducibility of the algorithm using clinically-relevant PROM data collected at either 12- or 24-months follow-up and support the improved utility of the refined desirability analysis algorithm to select MSC(M) donors for knee OA therapy. This serves as a useful multivariate tool to rank individual MSC(M) donors in terms of their basal immunomodulatory fitness, informed by clinical efficacy data out to 24 months follow-up in knee OA patients.

### Discussion

The data strongly supports the approach reported in Example 1 to empirically rank the basal immunomodulatory fitness of MSCs based on multivariate CQA gene expression readouts. The utility of these immunomodulatory CQA genes in MSC(M) was validated after initially identifying them for MSC(AT), and therefore, this supports the use for MSCs derived from both adipose and bone marrow tissue sources and other similar tissue sources such as mesenchymal stromal cells from dental pulp, synovium, placenta, and umbilical cord (for example umbilical cord blood and umbilical cord tissue (i.e., Wharton's jelly)). Notably, only a subset of the previously identified gene panel was measured, and all genes were measured under pro-inflammatory licensing conditions (no unlicensed condition).

Using the desirability ranking algorithm for MSC(AT) in Example 1, and the associated MSC(AT) data as a training dataset, the same algorithm ranked clinically efficacious MSC(M) donors higher in terms of their basal fitness.

A refined algorithm was developed to rank individual MSC(M) donors for knee OA therapy that utilized clinically-relevant PROM data into the algorithm. This provided proof-of-concept data showing that desirability scores were higher for MSC(M) derived from donors classified as responders versus non-responders in the knee OA clinical trial

Importantly, the significant differences between responders and non-responders and the significant positive correlations to PROM improvements is an expected result given that PROM data was used to inform the input parameters for the desirability ranking algorithm. Therefore, this new desirability ranking algorithm can be considered as a new “training dataset” for empirically ranking individual MSC(M) donors based on their clinical efficacy in knee OA. The algorithm can continue to be refined using data from larger controlled clinical trials.

Taken together, this work strongly supports the use of CQA genes to measure the basal immunomodulatory fitness of MSCs in general, and MSC(AT) and MSC(M) specifically. Example 1 showed that these CQAs were sensitive to varying CPP conditions for MSC(AT), and the inventors have further demonstrated their sensitivity using nine different MSC(M) donors as an alternative tissue source. Furthermore, the inventors presented a refined desirability ranking algorithm informed by clinical efficacy data that can be used to screen prospective MSC donors and/or CPP conditions to select MSCs with high basal immunomodulatory fitness that are fit-for-purpose in MSC-treatable disease therapy, such as knee OA therapy.

While the present disclosure has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present description is found to be defined differently in a document incorporated herein by reference, the definition provided herein is to serve as the definition for the term.

TABLE 1
Summary of MSC(AT) donor characteristics. Subcutaneous adipose
tissue was collected from human donors. Depot column indicates
anatomical location of adipose tissue collection. Procedure
column indicates the procedure the donor underwent for the
adipose tissue collection. All patients (except D1) had been
diagnosed with knee or hand osteoarthritis. M: Male, F: Female,
BMI: Body Mass Index, KL: Kellgren-Lawrence grade.

| | | | | | | Osteo- | |
|---|---|---|---|---|---|---|---|
| Donor | | | | | | arthritis | KL |
| ID | Sex | Age | BMI | Depot | Procedure | location | grade |
| D1 | M | 46 | 27.3 | Knee | Arthroscopy | None | N/A |
| D2 | F | 38 | 22.0 | Knee | Arthroscopy | Knee | 2 |
| D3 | M | 28 | 26.9 | Knee | Arthroscopy | Knee | 1 |
| D4 | F | 52 | 22.4 | Knee | Arthroscopy | Knee | 1 |
| D5 | M | 54 | 30.1 | Abdomen | Lipoaspirate | Hand | N/A |

TABLE 2
MSC(AT) cultured using 3D-N and 2D-H conditions satisfied
surface marker expression criteria by IFATS and ISCT. Single
cell suspensions of MSC(AT) display expression of positive
MSC(AT) markers and lack expression of the variable marker
CD34 as well as hematopoietic markers. Enzymatic digestion
conditions used for dissociating 3D-N MSC(AT) also cleave
CD105 from 2D-N MSC(AT) (2D-N Digest condition).

| | | | | | 2D-N |
|---|---|---|---|---|---|
| | | | | | (Di- |
| | | 3D-N | 2D-H | 2D-N | gest)) |
| Positive | CD90 | 97.40 ± 2.18 | 98.43 ± 0.71 | 99.10 ± 0.66 | — |
| markers | CD73 | 99.83 ± 0.12 | 99.87 ± 0.15 | 99.83 ± 0.21 | |
| | CD44 | 99.80 ± 0.17 | 99.80 ± 0.26 | 99.80 ± 0.26 | |
| | CD29 | 99.87 ± 0.06 | 99.83 ± 0.21 | 99.90 ± 0.17 | |
| | CD13 | 99.90 ± 0.10 | 99.87 ± 0.15 | 99.83 ± 0.21 | |
| | CD105 | 5.58 ± 6.13 | 93.97 ± 5.17 | 93.60 ± 6.06 | 0.17 |
| Variable | CD34 | 0.84 ± 0.32 | 1.46 ± 0.57 | 1.52 ± 0.52 | — |
| Negative | CD31 | 0.33 ± 0.28 | 0.71 ± 0.37 | 0.65 ± 0.34 | |
| markers | CD45 | 0.13 ± 0.10 | 0.11 ± 0.05 | 0.06 ± 0.07 | |

N = 3 MSC(AT) donors (two donors from subcutaneous knee fat, one abdominal lipoaspirate donor).
3D-N: 3D Normoxic culture, 2D-N: 2D Normoxic culture, 2D-H: 2D Hypoxic culture.
Data represented as mean ± standard deviation.

TABLE 3
Putative CQAs for MSC(AT) immunomodulatory fitness. Summary of regression analyses
between MΦ PC1 composite scores and MSC(AT) genes, soluble factors, and morphological
features. Marker/Characteristic column indicates the gene or protein symbol, or
morphological feature. Licensed/Unlicensed column indicates whether the factor was
measured under licensed or unlicensed conditions. Min/Max Function column indicates
whether minimization or maximation of the MSC(AT) characteristic was desirable based
on positive or negative correlations in the linear regression analysis. Min value
and Max value columns indicate the range of values measured across each gene (units:
mRNA count), soluble factor (units: pg/mL), and morphological feature (units for
cell diameter: microns, units for cell circularity: arbitrary). All significant
(*p < 0.05) and near-significant (p < 0.1) correlations are displayed.

| Marker/ | Licensed/ | Gene/ | Min/Max | Min | Max | p | R² |
|---|---|---|---|---|---|---|---|
| Characteristic | Unlicensed | protein | Function | value | value | value | value |
| THBS1 | Licensed | Gene | Min | 296.30 | 1680.04 | *0.0025 | 0.5481 |
| CCN2 | Unlicensed | Gene | Min | 289.15 | 11393.39 | *0.0035 | 0.4934 |
| TSG101 | Unlicensed | Gene | Min | 209.22 | 599.97 | *0.0087 | 0.4229 |
| THBS1 | Unlicensed | Gene | Min | 575.38 | 15145.48 | *0.0135 | 0.3853 |
| PDGFA | Unlicensed | Gene | Min | 1.00 | 127.95 | *0.0152 | 0.3754 |
| Cell Diameter | N/A | N/A | Min | 18.00 | 24.70 | *0.0199 | 0.3515 |
| CCN2 | Licensed | Gene | Min | 63.40 | 634.06 | *0.0418 | 0.302 |
| VEGFA | Unlicensed | Gene | Min | 214.27 | 894.31 | *0.0378 | 0.2914 |
| EDN1 | Licensed | Gene | Min | 1.00 | 65.27 | *0.0491 | 0.2854 |
| EDIL3 | Unlicensed | Gene | Min | 31.41 | 887.90 | *0.0477 | 0.2689 |
| ACTA2 | Licensed | Gene | Min | 54.88 | 201.10 | 0.0614 | 0.2619 |
| PDCD1LG2 | Licensed | Gene | Min | 47.65 | 329.96 | 0.0628 | 0.2595 |
| TNFAIP6 | Licensed | Gene | Max | 284.00 | 2300.07 | 0.0742 | 0.2417 |
| ACTA2 | Unlicensed | Gene | Min | 78.54 | 3197.28 | 0.0736 | 0.2256 |
| TGFβ | Unlicensed | Protein | Max | 85.54 | 1292.81 | 0.0865 | 0.2251 |
| ANGPT1 | Licensed | Gene | Min | 42.27 | 237.48 | 0.0868 | 0.2247 |
| ANGPT1 | Unlicensed | Gene | Min | 27.79 | 327.12 | 0.0749 | 0.2238 |
| CXCL8 | Licensed | Gene | Min | 25008.42 | 51553.32 | 0.0946 | 0.2154 |
| ANG | Unlicensed | Gene | Min | 25.10 | 88.99 | 0.0861 | 0.2096 |
| Cell Circularity | N/A | N/A | Max | 0.47 | 0.77 | 0.0981 | 0.1964 |

TABLE 4
Putative CQAs for MSC(AT) angiogenic fitness. Summary of regression analyses between
HUVEC PC1 composite scores and MSC(AT) genes, soluble factors, and morphological
features. Marker/Characteristic column indicates the gene or protein symbol, or
morphological feature. Licensed/Unlicensed column indicates whether the factor
was measured under licensed or unlicensed conditions. Min/Max Function column
indicates whether minimization or maximation of the MSC(AT) characteristic was
desirable based on positive or negative correlations in the linear regression
analysis. Min value and Max value columns indicate the range of values measured
across each gene (units: mRNA count) and soluble factor (units: pg/mL). All significant
(*p < 0.05) and near-significant correlations (p < 0.01) are displayed.

| Marker/ | Licensed/ | Gene/ | Min/Max | Min | Max | p | R² |
|---|---|---|---|---|---|---|---|
| Characteristic | Unlicensed | protein | Function | value | value | value | value |
| SMAD7 | Licensed | Gene | Min | 194.72 | 504.28 | *0.0117 | 0.4237 |
| HGF | Licensed | Gene | Min | 139.8 | 4151.35 | *0.0187 | 0.3807 |
| ANG | Licensed | Gene | Min | 1 | 61.01 | *0.0285 | 0.3404 |
| VEGF | Licensed | Protein | Max | 15.2 | 942.15 | *0.0328 | 0.3048 |
| SOX9 | Unlicensed | Gene | Min | 1 | 61.46 | *0.0381 | 0.2906 |
| IDO1 | Licensed | Gene | Min | 4326.67 | 17824.17 | 0.0501 | 0.2832 |
| VEGF | Unlicensed | Protein | Max | 250.575 | 1463.65 | 0.0743 | 0.2416 |
| TSG101 | Licensed | Gene | Min | 272.06 | 488.06 | 0.0744 | 0.2413 |
| CXCL8 | Licensed | Gene | Min | 25008.42 | 51553.32 | 0.0879 | 0.2234 |
| TNFAIP6 | Unlicensed | Gene | Min | 1 | 45.68 | 0.0809 | 0.216 |

TABLE 5
Forward and reverse primer sequences for qPCR.

| | | SEQ | | SEQ |
|---|---|---|---|---|
| | | ID | | ID |
| Gene | Forward | NO: | Reverse | NO: |
| ACTB | AGAGGGAAAT | 1 | AGGAGCCAGG | 2 |
| | CGTGCGTGAC | | GCAGTAATC | |
| B2M | CTCCGTGGCC | 3 | TTTGGAGTAC | 4 |
| | TTAGCTGTG | | GCTGGATAGC | |
| | | | CT | |
| CCN2 | TGTGGCTTTA | 5 | GCTACAGGCA | 6 |
| | GGAGCAGTGG | | GGTCAGTGAG | |
| CCR7 | TTTTACCGCC | 7 | AATGACAAGG | 8 |
| | CAGAGAGCG | | AGAGCCACC | |
| CD163 | TGGACCTAAT | 9 | ACACAGAAAT | 10 |
| | GAATTCCTCA | | TAGTTCAGCA | |
| | GAAAA | | GCA | |
| CD206 | CTACAAGGGA | 11 | TTGGCATTGC | 12 |
| | TCGGGTTTAT | | CTAGTAGCGT | |
| | GGA | | A | |
| CD274 | TCAATGCCCC | 13 | TGCTTGTCCA | 14 |
| | ATACAACAA | | GATGACTTCG | |
| CD86 | CTGCTCATCT | 15 | GGAAACGTCG | 16 |
| | ATACACGGTT | | TACAGTTCTG | |
| | ACC | | TG | |
| CD86 | CCATCAGCTT | 17 | GCTGTAATCC | 18 |
| | GTCTGTTTCA | | AAGGAATGTG | |
| | TTCC | | GTC | |
| HGF | TGGTTTTAAT | 19 | GAGATGTGCC | 20 |
| | GAAGCTTGCC | | ACTCGTAATA | |
| | AG | | GG | |
| HLADRA | AAGCACTGGG | 21 | ATTGCTTTTG | 22 |
| | AGTTTGATGC | | CGCAATCCCT | |
| HMOX1 | AAGACTGCGT | 23 | AAAGCCCTAC | 24 |
| | TCCTGCTCAA | | AGCAACTGTC | |
| | C | | G | |
| IDO1 | GCCCTTCAAG | 25 | CCAGCCAGAC | 26 |
| | TGTTTCACCA | | AAATATATGC | |
| | A | | GA | |
| IL10 | CGAGATGCCT | 27 | CGCCTTGATG | 28 |
| | TCAGCAGAGT | | TCTGGGTCTT | |
| IL12A | CCTCCACTGT | 29 | TCAGCAACAT | 30 |
| | GCTGGTTTTA | | GCTCCAGAAG | |
| | T | | | |
| IL1B | GTACCTGTCC | 31 | GGGAACTGGG | 32 |
| | TGCGTGTTGA | | CAGACTCAAA | |
| IL1RA | AGCATGAGGC | 33 | AAATCCAGCA | 34 |
| | TCAATGGGTA | | AGATGCAAGC | |
| PRG4 | AGGCCCCATG | 35 | GCGCAAAGTA | 36 |
| | TGTTCATGC | | GTCAGTCCAT | |
| | | | CT | |
| PTGS2 | ATAAGCGAGG | 37 | CGCAGTTTAC | 38 |
| | GCCAGCTTTC | | GCTGTCTAGC | |
| RPL13A | TGCGACAAAA | 39 | TGTTGATGCC | 40 |
| | CCTCCTCCTT | | TTCACAGCGT | |
| | | | A | |
| STAB1 | GAACCATGTG | 41 | AGCGGAATCT | 42 |
| | CCACTGGAAG | | CCTGGTGCAG | |
| | GC | | TT | |
| TBP | AGCGCAAGGG | 43 | AATAGGCTGT | 44 |
| | TTTCTGGTTT | | GGGGTCAGTC | |
| TNFAIP6 | AGCACGGTCT | 45 | ATCCATCCAG | 46 |
| | GGCAAATACA | | CAGCACAGAC | |
| TREM1 | AGTTGCAGCT | 47 | GAACCATGTG | 48 |
| | CGGAGTTCTG | | CCACTGGAAG | |
| | AGACA | | GC | |
| VEGFA | CTACCTCCAC | 49 | AGCTGCGCTG | 50 |
| | CATGCCAAGT | | ATAGACATCC | |

TABLE 6
Gene expression measured for MSC(AT) under licensed conditions. All data values represent
mRNA counts. 2D-N: 2D normoxic, 2D-H: 2D hypoxic, 3D-N: 3D normoxic, D: donor.

| | 2D-N | | | | 2D-H | | | |
|---|---|---|---|---|---|---|---|---|
| | D1 | D2 | D3 | D4 | D1 | D2 | D3 | D4 |
| ACTA2 | 66.71 | 73.01 | 159.42 | 126.59 | 54.88 | 59.98 | 201.10 | 104.78 |
| ANG | 1.00 | 29.78 | 31.29 | 49.80 | 27.05 | 27.93 | 46.31 | 45.26 |
| ANGPT1 | 69.55 | 68.21 | 215.51 | 60.18 | 77.29 | 50.94 | 237.48 | 105.62 |
| CCN2 | 370.44 | 634.06 | 366.07 | 414.00 | 175.45 | 315.50 | 416.75 | 274.10 |
| CD274 | 55.35 | 60.52 | 71.44 | 179.51 | 81.93 | 117.49 | 160.09 | 202.01 |
| CXCL8 | 33604.11 | 31658.65 | 31881.42 | 51553.32 | 31885.05 | 25841.33 | 44656.26 | 49225.02 |
| EDIL3 | 33.35 | 268.03 | 240.90 | 44.62 | 38.64 | 188.15 | 302.97 | 64.54 |
| EDN1 | 1.00 | 28.82 | 34.24 | 63.29 | 35.55 | 1.00 | 63.51 | 59.51 |
| F3 | 132.70 | 269.95 | 1253.48 | 482.49 | 192.45 | 188.97 | 1602.85 | 781.22 |
| HGF | 139.80 | 373.71 | 774.05 | 1322.94 | 191.68 | 569.38 | 1041.89 | 1590.93 |
| HLA-DRA | 58.90 | 91.27 | 303.48 | 227.24 | 40.19 | 52.58 | 279.82 | 93.04 |
| ICAM1 | 1724.45 | 2976.22 | 3400.87 | 5953.77 | 2317.14 | 3178.82 | 4966.00 | 6604.30 |
| IDO1 | 6089.52 | 6800.74 | 4326.67 | 12426.34 | 7418.26 | 6853.89 | 5440.30 | 13258.88 |
| NFKBIA | 1167.38 | 1195.10 | 1092.29 | 2003.61 | 1294.60 | 1262.00 | 1598.22 | 2450.10 |
| NOS2 | 1.00 | 1.00 | 1.00 | 1.00 | 1.00 | 1.00 | 1.00 | 1.00 |
| PDCD1LG2 | 267.54 | 244.98 | 190.12 | 329.96 | 258.15 | 227.59 | 288.42 | 231.35 |
| PDGFA | 1.00 | 1.00 | 31.88 | 32.17 | 1.00 | 1.00 | 37.71 | 1.00 |
| PRG4 | 1.00 | 1.00 | 1.00 | 1.00 | 1.00 | 1.00 | 1.00 | 1.00 |
| PTGES | 965.13 | 890.56 | 1451.28 | 1562.63 | 908.93 | 955.53 | 2055.99 | 1638.71 |
| PTGS2 | 709.65 | 865.58 | 1409.35 | 1610.36 | 1385.80 | 1561.06 | 3568.88 | 3367.11 |
| SMAD7 | 268.25 | 275.72 | 217.87 | 504.28 | 265.10 | 194.72 | 267.25 | 398.99 |
| TGFB1 | 97.93 | 96.07 | 78.53 | 169.13 | 119.03 | 270.31 | 219.62 | 267.39 |
| THBS1 | 2619.32 | 7517.42 | 5645.69 | 9328.05 | 2541.28 | 6022.41 | 6726.29 | 4569.95 |
| THBS2 | 943.13 | 333.36 | 1221.60 | 1577.16 | 1084.37 | 296.60 | 1349.49 | 1344.50 |
| TIMP1 | 3559.61 | 2387.32 | 4687.42 | 3605.67 | 3938.68 | 2026.09 | 6256.61 | 4231.31 |
| TLR2 | 72.38 | 410.22 | 161.78 | 1039.68 | 129.85 | 736.99 | 419.40 | 1317.67 |
| TLR3 | 51.80 | 83.58 | 73.21 | 63.29 | 57.97 | 59.98 | 61.52 | 35.21 |
| TLR4 | 1.00 | 30.74 | 30.11 | 1.00 | 1.00 | 1.00 | 38.37 | 1.00 |
| TNFAIP6 | 390.31 | 525.50 | 284.00 | 892.34 | 487.70 | 707.41 | 608.59 | 1015.92 |
| TSG101 | 297.34 | 338.16 | 311.75 | 437.87 | 272.06 | 285.10 | 421.39 | 424.98 |
| TWIST1 | 1.00 | 1.00 | 1.00 | 28.02 | 1.00 | 40.26 | 59.54 | 1.00 |
| VEGFA | 300.89 | 215.20 | 210.78 | 373.54 | 319.98 | 234.16 | 389.63 | 517.18 |

| | 2D-H | 3D-N | | | | |
|---|---|---|---|---|---|---|
| | D5 | D1 | D2 | D3 | D4 | D5 |
| ACTA2 | 182.93 | 64.57 | 61.05 | 73.53 | 129.03 | 130.04 |
| ANG | 58.40 | 1.00 | 32.76 | 33.61 | 61.01 | 60.46 |
| ANGPT1 | 79.87 | 42.27 | 61.05 | 204.84 | 94.67 | 104.95 |
| CCN2 | 470.63 | 63.40 | 95.29 | 96.64 | 214.58 | 295.45 |
| CD274 | 196.67 | 136.19 | 92.32 | 102.95 | 155.68 | 242.98 |
| CXCL8 | 46010.56 | 26878.33 | 31301.07 | 27422.28 | 37158.81 | 25008.42 |
| EDIL3 | 287.71 | 149.10 | 564.32 | 571.45 | 122.02 | 400.40 |
| EDN1 | 65.27 | 1.00 | 1.00 | 1.00 | 1.00 | 45.63 |
| F3 | 1752.86 | 194.89 | 737.04 | 1655.53 | 1138.82 | 2779.98 |
| HGF | 380.46 | 801.87 | 1926.72 | 2294.21 | 4151.35 | 1639.24 |
| HLA-DRA | 190.66 | 241.85 | 608.99 | 2322.57 | 1131.80 | 2098.96 |
| ICAM1 | 7610.89 | 3950.62 | 7120.24 | 6305.92 | 8007.48 | 10232.43 |
| IDO1 | 9205.72 | 12371.97 | 13193.74 | 9266.12 | 17824.17 | 14762.30 |
| NFKBIA | 2114.42 | 2472.52 | 3071.74 | 2177.61 | 3567.22 | 3125.62 |
| NOS2 | 1.00 | 31.70 | 52.11 | 183.83 | 52.59 | 71.87 |
| PDCD1LG2 | 322.06 | 79.83 | 47.65 | 59.88 | 63.81 | 95.82 |
| PDGFA | 33.49 | 1.00 | 28.29 | 37.82 | 32.26 | 38.79 |
| PRG4 | 1.00 | 95.10 | 41.69 | 119.75 | 55.40 | 41.07 |
| PTGES | 1709.06 | 2157.87 | 2499.98 | 3088.35 | 3586.85 | 3151.86 |
| PTGS2 | 693.93 | 2112.09 | 3566.08 | 2674.47 | 4453.59 | 862.40 |
| SMAD7 | 320.34 | 363.95 | 454.14 | 298.33 | 502.79 | 436.90 |
| TGFB1 | 188.94 | 206.63 | 227.81 | 118.70 | 297.33 | 246.40 |
| THBS1 | 6013.48 | 860.57 | 1745.07 | 512.62 | 1135.31 | 2015.69 |
| THBS2 | 1368.96 | 1680.04 | 296.30 | 917.05 | 1403.19 | 1511.48 |
| TIMP1 | 5595.23 | 7416.37 | 7633.94 | 10374.35 | 10542.47 | 12197.92 |
| TLR2 | 1727.95 | 516.57 | 1682.53 | 774.19 | 1865.30 | 2882.65 |
| TLR3 | 37.79 | 104.49 | 86.36 | 60.93 | 59.61 | 62.74 |
| TLR4 | 30.06 | 30.52 | 52.11 | 58.83 | 35.06 | 76.43 |
| TNFAIP6 | 692.21 | 1266.78 | 2147.09 | 894.99 | 2300.07 | 1692.86 |
| TSG101 | 420.82 | 407.39 | 400.53 | 352.95 | 488.06 | 473.41 |
| TWIST1 | 27.48 | 1.00 | 28.29 | 54.62 | 63.81 | 54.76 |
| VEGFA | 453.46 | 231.28 | 226.32 | 130.26 | 258.76 | 198.49 |

TABLE 7
Gene expression measured for MSC(AT) under unlicensed conditions. All data values represent
mRNA counts. 2D-N: 2D normoxic, 2D-H: 2D hypoxic, 3D-N: 3D normoxic, D: donor.

| | 2D-N | | | | | 2D-H | | |
|---|---|---|---|---|---|---|---|---|
| | D1 | D2 | D3 | D4 | D5 | D1 | D2 | D3 |
| ACTA2 | 103.34 | 280.09 | 2470.80 | 292.80 | 528.41 | 78.54 | 269.85 | 3197.28 |
| ANG | 27.23 | 33.98 | 68.28 | 60.31 | 88.99 | 32.04 | 25.10 | 58.03 |
| ANGPT1 | 37.01 | 33.98 | 204.19 | 92.05 | 114.98 | 60.95 | 27.79 | 170.28 |
| CCN2 | 3211.24 | 6617.70 | 11393.39 | 5027.57 | 3347.68 | 970.09 | 3834.39 | 7376.29 |
| CXCL8 | 488.08 | 314.89 | 230.04 | 250.74 | 458.33 | 98.01 | 158.68 | 76.10 |
| EDIL3 | 32.12 | 256.06 | 763.71 | 66.65 | 378.00 | 31.41 | 242.96 | 725.83 |
| EDN1 | 1.00 | 42.26 | 53.70 | 1.00 | 18.11 | 1.00 | 53.79 | 54.22 |
| F3 | 300.95 | 237.83 | 1079.94 | 556.24 | 1270.24 | 807.36 | 346.95 | 1278.53 |
| HGF | 43.29 | 91.15 | 653.66 | 168.22 | 273.26 | 37.07 | 83.38 | 554.60 |
| ICAM1 | 29.33 | 45.58 | 45.08 | 46.02 | 103.95 | 15.71 | 1.00 | 26.64 |
| NFKBIA | 191.32 | 223.74 | 325.51 | 280.10 | 384.30 | 559.19 | 259.99 | 315.83 |
| PDCD1LG2 | 83.09 | 72.09 | 85.52 | 115.06 | 140.18 | 96.13 | 86.07 | 109.40 |
| PDGFA | 1.00 | 48.06 | 127.95 | 61.89 | 66.94 | 1.00 | 52.00 | 103.69 |
| PRG4 | 1.00 | 1.00 | 60.33 | 1.00 | 1.00 | 1.00 | 1.00 | 42.81 |
| PTGS2 | 62.14 | 35.63 | 49.06 | 67.45 | 92.14 | 324.83 | 94.13 | 43.76 |
| SMAD7 | 111.02 | 140.04 | 322.85 | 207.10 | 244.91 | 94.87 | 90.55 | 253.04 |
| SOX9 | 50.97 | 29.83 | 1.00 | 53.16 | 1.00 | 31.41 | 33.17 | 1.00 |
| TGFB1 | 132.67 | 200.54 | 423.62 | 322.16 | 232.31 | 140.11 | 487.70 | 420.47 |
| THBS1 | 4010.04 | 13388.71 | 10911.43 | 14430.46 | 7249.76 | 2613.10 | 9433.12 | 12694.95 |
| THBS2 | 553.01 | 674.53 | 4893.20 | 868.08 | 2294.00 | 404.62 | 478.74 | 4279.85 |
| TIMP1 | 4588.19 | 3941.12 | 13179.36 | 7460.42 | 10995.12 | 4283.74 | 3655.99 | 11755.08 |
| TLR2 | 1.00 | 1.00 | 1.00 | 1.00 | 73.24 | 1.00 | 1.00 | 1.00 |
| TLR3 | 1.00 | 1.00 | 45.08 | 1.00 | 25.20 | 1.00 | 1.00 | 34.25 |
| TLR4 | 1.00 | 36.46 | 71.60 | 30.15 | 55.91 | 28.90 | 42.14 | 73.25 |
| TNFAIP6 | 1.00 | 1.00 | 1.00 | 28.57 | 1.00 | 1.00 | 1.00 | 29.49 |
| TSG101 | 238.80 | 299.98 | 599.97 | 532.43 | 504.79 | 209.22 | 284.20 | 560.31 |
| TWIST1 | 1.00 | 1.00 | 55.69 | 38.88 | 40.95 | 33.30 | 104.89 | 91.32 |
| VASH1 | 1.00 | 1.00 | 29.83 | 26.19 | 1.00 | 1.00 | 1.00 | 30.44 |
| VEGFA | 500.64 | 367.93 | 894.31 | 710.97 | 678.83 | 461.80 | 214.27 | 761.98 |

| | 2D-H | | 3D-N | | | | |
|---|---|---|---|---|---|---|---|
| | D4 | D5 | D1 | D2 | D3 | D4 | D5 |
| ACTA2 | 345.23 | 905.41 | 169.22 | 146.93 | 512.59 | 456.80 | 395.51 |
| ANG | 65.62 | 70.28 | 28.49 | 44.36 | 55.49 | 71.43 | 65.28 |
| ANGPT1 | 94.15 | 118.52 | 45.76 | 41.58 | 327.12 | 96.34 | 93.44 |
| CCN2 | 3600.68 | 3836.61 | 426.51 | 586.33 | 289.15 | 452.65 | 334.07 |
| CXCL8 | 233.96 | 250.81 | 609.55 | 298.01 | 299.38 | 434.37 | 350.71 |
| EDIL3 | 85.59 | 475.44 | 37.13 | 212.08 | 887.90 | 58.14 | 355.83 |
| EDN1 | 1.00 | 1.00 | 1.00 | 1.00 | 1.00 | 1.00 | 1.00 |
| F3 | 1015.72 | 1802.54 | 290.10 | 238.41 | 940.48 | 826.39 | 2017.25 |
| HGF | 92.25 | 165.37 | 133.82 | 224.55 | 1380.05 | 320.59 | 400.63 |
| ICAM1 | 74.18 | 110.25 | 118.28 | 180.19 | 242.42 | 245.01 | 181.76 |
| NFKBIA | 548.76 | 569.15 | 305.64 | 313.26 | 479.00 | 498.33 | 559.35 |
| PDCD1LG2 | 127.44 | 162.61 | 58.71 | 37.43 | 33.59 | 47.34 | 37.12 |
| PDGFA | 38.04 | 74.42 | 1.00 | 59.60 | 113.91 | 67.27 | 85.76 |
| PRG4 | 1.00 | 1.00 | 102.74 | 29.11 | 497.98 | 60.63 | 78.08 |
| PTGS2 | 337.62 | 226.01 | 98.43 | 67.92 | 78.86 | 52.32 | 40.96 |
| SMAD7 | 204.48 | 224.63 | 249.52 | 246.73 | 448.33 | 427.73 | 450.55 |
| SOX9 | 38.04 | 1.00 | 30.22 | 36.04 | 1.00 | 61.46 | 29.44 |
| TGFB1 | 352.84 | 227.39 | 212.39 | 559.99 | 480.46 | 581.38 | 343.03 |
| THBS1 | 15145.48 | 12391.80 | 1009.29 | 2080.55 | 575.38 | 6109.48 | 2771.16 |
| THBS2 | 780.81 | 2253.18 | 945.40 | 1388.88 | 6546.82 | 1918.56 | 3143.63 |
| TIMP1 | 7918.45 | 11516.71 | 10140.39 | 11963.53 | 39473.71 | 23333.31 | 30155.04 |
| TLR2 | 1.00 | 67.53 | 1.00 | 44.36 | 26.29 | 46.51 | 206.08 |
| TLR3 | 1.00 | 26.18 | 1.00 | 26.34 | 167.94 | 1.00 | 1.00 |
| TLR4 | 53.26 | 103.36 | 25.90 | 27.72 | 112.45 | 32.39 | 88.32 |
| TNFAIP6 | 35.19 | 1.00 | 32.81 | 1.00 | 45.27 | 45.68 | 40.96 |
| TSG101 | 454.60 | 463.04 | 255.56 | 339.60 | 579.77 | 533.21 | 463.35 |
| TWIST1 | 55.16 | 38.59 | 1.00 | 31.88 | 58.41 | 44.85 | 48.64 |
| VASH1 | 31.38 | 37.21 | 1.00 | 1.00 | 75.94 | 61.46 | 47.36 |
| VEGFA | 565.88 | 712.47 | 683.80 | 368.71 | 756.47 | 498.33 | 481.27 |

TABLE 8
Differential gene expression analysis relative to 2D-N measured under
licensed conditions. Multivariate linear regression, Benjamini-Yekutieli
(BY) False Discovery Rate-corrected p values. Bolded font indicates
p < 0.05 vs 2D-N. 3D-N: 3D Normoxic culture, 2D-N: 2D Normoxic
culture, 2D-H: 2D Hypoxic culture. N = 5 MSC(AT) donors, n =
1 technical replicate due to high sensitivity of Nanostring measurements.

| | 3D-N vs 2D-N | | | 2D-H vs 2D-N | | |
|---|---|---|---|---|---|---|
| | Log₂ Fold | Standard | BY p | Log₂ Fold | Standard | BY p |
| | Change | Error (Log₂) | value | Change | Error (Log₂) | value |
| ACTA2 | 0.0416 | 0.187 | 1 | −0.16 | 0.189 | 1 |
| ANG | 0.595 | 0.178 | 0.0273 | 0.0481 | 0.178 | 1 |
| ANGPT1 | 0.422 | 0.201 | 0.28 | 0.0153 | 0.201 | 1 |
| CCN2 | −1.61 | 0.202 | 2.09E−05 | −0.851 | 0.199 | 0.0291 |
| CD274 | 0.965 | 0.25 | 0.0104 | 0.447 | 0.252 | 1 |
| CXCL8 | 0.141 | 0.216 | 1 | −0.0014 | 0.216 | 1 |
| EDIL3 | 1.9 | 0.237 | 2.08E−05 | 0.165 | 0.244 | 1 |
| EDN1 | −1.05 | 0.259 | 0.00751 | 0.0257 | 0.23 | 1 |
| F3 | 1.37 | 0.24 | 0.000407 | 0.255 | 0.242 | 1 |
| HGF | 2.47 | 0.214 | 6.09E−07 | 0.208 | 0.216 | 1 |
| HLA-DRA | 2.99 | 0.192 | 4.36E−08 | −0.879 | 0.207 | 0.0291 |
| ICAM1 | 1.37 | 0.228 | 0.000249 | 0.209 | 0.228 | 1 |
| IDO1 | 1.34 | 0.211 | 0.000147 | 0.0651 | 0.211 | 1 |
| NOS2 | 6.07 | 0.454 | 1.68E−07 | 1.52 | 0.454 | 0.14 |
| NFKBIA | 1.51 | 0.199 | 3.08E−05 | 0.203 | 0.199 | 1 |
| PDGFA | 1.34 | 0.625 | 0.27 | −0.161 | 0.625 | 1 |
| PDCD1LG2 | −1.65 | 0.216 | 3.07E−05 | −0.213 | 0.208 | 1 |
| PRG4 | 3.56 | 0.316 | 6.09E−07 | −0.479 | 0.419 | 1 |
| PTGES | 1.68 | 0.173 | 2.82E−06 | 0.0833 | 0.174 | 1 |
| PTGS2 | 1.93 | 0.215 | 6.33E−06 | 0.977 | 0.215 | 0.0291 |
| SMAD7 | 0.819 | 0.232 | 0.0191 | −0.256 | 0.234 | 1 |
| TGFB1 | 1.42 | 0.189 | 3.12E−05 | 0.823 | 0.191 | 0.0291 |
| THBS1 | −2.18 | 0.301 | 4.10E−05 | −0.582 | 0.301 | 1 |
| THBS2 | 0.421 | 0.212 | 0.336 | −0.193 | 0.213 | 1 |
| TIMP1 | 1.82 | 0.178 | 1.71E−06 | 0.0917 | 0.178 | 1 |
| TLR2 | 2.32 | 0.311 | 3.23E−05 | 0.663 | 0.312 | 0.839 |
| TLR3 | 0.728 | 0.162 | 0.00348 | −0.502 | 0.181 | 0.309 |
| TLR4 | 1.59 | 0.249 | 0.000142 | −0.0289 | 0.249 | 1 |
| TSG101 | 0.697 | 0.164 | 0.00533 | −0.0988 | 0.166 | 1 |
| TNFAIP6 | 2.14 | 0.189 | 6.09E−07 | 0.365 | 0.191 | 1 |
| TWIST1 | 1.98 | 0.28 | 4.86E−05 | 0.849 | 0.299 | 0.309 |
| VEGFA | 0.0164 | 0.22 | 1 | 0.348 | 0.219 | 1 |

TABLE 9
Differential gene expression analysis relative to 2D-N measured under
unlicensed conditions. Multivariate linear regression, Benjamini-Yekutieli
(BY) False Discovery Rate-corrected p values. Bolded font indicates
p < 0.05 vs 2D-N. 3D-N: 3D Normoxic culture, 2D-N: 2D Normoxic
culture, 2D-H: 2D Hypoxic culture. N = 5 MSC(AT) donors, n =
1 technical replicate due to high sensitivity of Nanostring measurements.

| | 3D-N vs 2D-N | | | 2D-H vs 2D-N | | |
|---|---|---|---|---|---|---|
| | Log₂ Fold | Standard | BY p | Log₂ Fold | Standard | BY p |
| | Change | Error (Log₂) | value | Change | Error (Log₂) | value |
| ACTA2 | 0.174 | 0.334 | 1 | 0.726 | 0.333 | 0.625 |
| ANG | 0.626 | 0.163 | 0.0115 | 0.23 | 0.17 | 1 |
| ANGPT1 | 0.928 | 0.227 | 0.00794 | 0.463 | 0.231 | 0.704 |
| CCN2 | −3.07 | 0.296 | 1.76E−06 | −0.181 | 0.295 | 1 |
| CXCL8 | 0.918 | 0.453 | 0.266 | −0.859 | 0.455 | 0.736 |
| EDIL3 | 0.701 | 0.186 | 0.0124 | 0.551 | 0.187 | 0.157 |
| F3 | 0.829 | 0.205 | 0.00798 | 1.1 | 0.205 | 0.00596 |
| | 1.76 | 0.173 | 1.76E−06 | −0.0518 | 0.181 | 1 |
| ICAM1 | 2.79 | 0.459 | 0.000344 | 0.0797 | 0.47 | 1 |
| NFKBIA | 1.29 | 0.22 | 0.000448 | 1.07 | 0.22 | 0.00876 |
| PDGFA | 0.544 | 0.259 | 0.242 | −0.00801 | 0.259 | 1 |
| PDCD1LG2 | −0.621 | 0.325 | 0.318 | 0.67 | 0.317 | 0.635 |
| PRG4 | 3.88 | 0.527 | 6.00E−05 | −0.854 | 0.527 | 0.952 |
| PTGES | 3.67 | 0.64 | 0.000512 | 0.0319 | 0.724 | 1 |
| PTGS2 | 1 | 0.474 | 0.242 | 1.99 | 0.471 | 0.017 |
| SMAD7 | 1.53 | 0.147 | 1.76E−06 | 0.122 | 0.153 | 1 |
| SOX9 | 0.536 | 0.23 | 0.169 | −0.143 | 0.244 | 1 |
| TGFB1 | 1.38 | 0.133 | 1.76E−06 | 0.642 | 0.135 | 0.00876 |
| THBS1 | −1.7 | 0.301 | 0.00056 | 0.398 | 0.301 | 1 |
| THBS2 | 1.4 | 0.187 | 5.94E−05 | 0.154 | 0.188 | 1 |
| TIMP1 | 2.11 | 0.167 | 5.63E−07 | 0.308 | 0.167 | 0.736 |
| TLR2 | 2.64 | 0.518 | 0.00133 | 0.561 | 0.546 | 1 |
| TLR3 | 1.84 | 0.513 | 0.0172 | 0.027 | 0.54 | 1 |
| TLR4 | 1.14 | 0.23 | 0.00158 | 1.08 | 0.231 | 0.00876 |
| TSG101 | 0.655 | 0.129 | 0.00133 | 0.222 | 0.13 | 0.869 |
| TNFAIP6 | 1.94 | 0.306 | 0.000245 | 0.612 | 0.331 | 0.736 |
| TWIST1 | 0.865 | 0.249 | 0.0205 | 1.43 | 0.243 | 0.0046 |
| VASH1 | 1.85 | 0.265 | 9.63E−05 | 0.868 | 0.265 | 0.0937 |
| VEGFA | 0.493 | 0.212 | 0.169 | 0.148 | 0.212 | 1 |

TABLE 10
Soluble factor levels measured for MSC(AT) under licensed conditions. All data
values are in units of pg/mL. Values represent the mean of two technical replicate
wells. 2D-N: 2D normoxic, 2D-H: 2D hypoxic, 3D-N: 3D normoxic, D: donor.

| Culture | Donor | | | | | | | |
|---|---|---|---|---|---|---|---|---|
| Condition | ID | TGF-β | Ang1 | VEGF | HGF | PD-L2 | IL-1RA | PDGF-AA |
| 2D-N | D1 | 111.42 | 12.38 | 617.80 | 9.83 | 648.56 | 39.40 | 0.45 |
| | D2 | 107.33 | 16.98 | 368.48 | 37.00 | 529.37 | 39.40 | 3.07 |
| | D3 | 116.26 | 60.40 | 609.28 | 286.55 | 524.20 | 39.40 | 24.42 |
| | D4 | 593.73 | 9.10 | 74.35 | 28.65 | 591.77 | 39.40 | 1.64 |
| | D5 | 1013.52 | 6.75 | 15.20 | 4.40 | 452.24 | 23.10 | 1.01 |
| 2D-H | D1 | 78.23 | 18.03 | 726.55 | 7.30 | 708.86 | 61.78 | 1.56 |
| | D2 | 103.06 | 17.70 | 433.30 | 6.63 | 550.62 | 39.40 | 10.13 |
| | D3 | 69.23 | 80.95 | 942.15 | 77.58 | 520.01 | 39.40 | 58.86 |
| | D4 | 1738.44 | 22.25 | 64.05 | 179.03 | 807.59 | 83.08 | 3.79 |
| | D5 | 2135.49 | 58.23 | 491.63 | 339.63 | 1002.03 | 62.10 | 14.66 |
| 3D-N | D1 | 1128.79 | 12.38 | 479.98 | 511.93 | 472.30 | 114.70 | 3.80 |
| | D2 | 1856.83 | 14.03 | 325.48 | 3551.93 | 434.52 | 373.38 | 28.55 |
| | D3 | 1517.53 | 70.70 | 576.18 | 5736.73 | 414.27 | 138.85 | 116.68 |
| | D4 | 2750.45 | 40.73 | 104.15 | 8082.70 | 528.44 | 541.48 | 21.73 |
| | D5 | 2417.04 | 46.03 | 226.15 | 3303.23 | 532.72 | 693.55 | 41.07 |

TABLE 11
Soluble factor levels measured for MSC(AT) under unlicensed conditions. All data
values are in units of pg/mL. Values represent the mean of two technical replicate
wells. 2D-N: 2D normoxic, 2D-H: 2D hypoxic, 3D-N: 3D normoxic, D: donor.

| Culture | Donor | | | | | | |
|---|---|---|---|---|---|---|---|
| Condition | ID | TGF-β | Ang1 | VEGF | HGF | PD-L2 | PDGF-AA |
| 2D-N | D1 | 138.48 | 19.18 | 1035.88 | 8.05 | 955.69 | 1.17 |
| | D2 | 85.54 | 14.35 | 350.88 | 20.30 | 427.44 | 5.30 |
| | D3 | 96.14 | 70.15 | 919.28 | 185.13 | 507.20 | 48.11 |
| | D4 | 289.42 | 18.78 | 250.58 | 7.00 | 532.60 | 2.48 |
| | D5 | 448.12 | 42.20 | 617.85 | 15.85 | 519.10 | 11.10 |
| 2D-H | D1 | 675.93 | 20.75 | 782.55 | 20.75 | 828.29 | 0.75 |
| | D2 | 752.90 | 29.13 | 420.65 | 135.30 | 534.55 | 6.25 |
| | D3 | 890.42 | 123.60 | 1463.65 | 965.05 | 765.77 | 50.08 |
| | D4 | 893.08 | 65.25 | 461.83 | 7.88 | 698.95 | 4.90 |
| | D5 | 1196.66 | 65.90 | 901.58 | 36.08 | 752.62 | 20.96 |
| 3D-N | D1 | 866.31 | 17.75 | 1252.80 | 207.33 | 501.52 | 4.18 |
| | D2 | 1098.27 | 13.80 | 555.98 | 449.48 | 424.14 | 44.59 |
| | D3 | 1070.39 | 88.05 | 1108.13 | 2682.58 | 420.43 | 177.28 |
| | D4 | 1292.81 | 34.68 | 817.40 | 509.60 | 420.42 | 25.04 |

TABLE 12
PC1 scores corresponding to technical replicates for each
culture condition and donor within experimental batch 1.

| Donor | Media | PC1 Score | Mean PC1 Score ± SD |
|---|---|---|---|
| | Positive Control | 7.504 | 5.086 ± 2.095 |
| | | 3.918 | |
| | | 3.836 | |
| | Negative Control | 0.331 | −0.363 ± 1.802 |
| | | −2.408 | |
| | | 0.990 | |
| | UCM Control | −1.552 | −1.562 ± 0.785 |
| | | −0.782 | |
| | | −2.351 | |
| D2 | 2D-N | −3.247 | −3.284 ± 0.922 |
| | | −4.224 | |
| | | −2.382 | |
| | 2D-H | 1.558 | 1.441 ± 0.102 |
| | | 1.367 | |
| | | 1.401 | |
| | 3D-N | 0.034 | −0.359 ± 1.962 |
| | | −2.487 | |
| | | 1.377 | |
| D4 | 2D-N | −7.571 | −7.850 ± 0.688 |
| | | −8.633 | |
| | | −7.346 | |
| | 2D-H | −6.674 | −4.065 ± 3.691 |
| | | −1.455 | |
| | 3D-N | −7.837 | −8.024 ± 0.265 |
| | | −8.212 | |

N = 5 MSC(AT) donors, n = 2-3 technical replicates/condition.
UCM: unconditioned medium, 3D-N: 3D Normoxic culture, 2D-N: 2D Normoxic culture, 2D-H: 2D Hypoxic culture.

TABLE 13
PC1 scores corresponding to technical replicates for each
culture condition and donor within experimental batch 2.

| Donor | Media | PC1 Score | Mean PC1 Score + SD |
|---|---|---|---|
| | Positive Control | 5.132 | 4.967 ± 0.840 |
| | | 4.056 | |
| | | 5.712 | |
| | Negative Control | −2.122 | −0.0759 ± 1.829 |
| | | 1.401 | |
| | | 0.493 | |
| | UCM Control | 5.535 | 2.209 ± 3.014 |
| | | −0.343 | |
| | | 1.436 | |
| D1 | 2D-N | −1.995 | 1.047 ± 2.657 |
| | | 2.915 | |
| | | 2.221 | |
| | 2D-H | −0.863 | −0.555 ± 1.311 |
| | | 0.883 | |
| | | −1.685 | |
| | 3D-N | 7.747 | 4.002 ± 3.390 |
| | | 1.143 | |
| | | 3.116 | |
| D3 | 2D-N | −2.701 | −0.520 ± 2.162 |
| | | 1.622 | |
| | | −0.480 | |
| | 2D-H | 0.169 | 0.228 ± 3.014 |
| | | 3.270 | |
| | | −2.756 | |
| | 3D-N | 2.258 | 0.223 ± 1.841 |
| | | −1.326 | |
| | | −0.262 | |
| D5 | 2D-N | 1.652 | 0.894 ± 0.659 |
| | | 0.581 | |
| | | 0.449 | |
| | 2D-H | 7.728 | 3.225 ± 3.967 |
| | | 1.703 | |
| | | 0.244 | |
| | 3D-N | −1.207 | −0.695 ± 1.853 |
| | | −2.239 | |
| | | 1.360 | |

N = 5 MSC(AT) donors, n = 2-3 technical replicates/condition.
UCM: unconditioned medium, 3D-N: 3D Normoxic culture, 2D-N: 2D Normoxic culture, 2D-H: 2D Hypoxic culture.

TABLE 14
Patients classified as Pain-Function Responders, Function Responders,
Pain Responders, or Non-Responders based on changes in KOOS pain and
ADL scores at 12 months relative to baseline. Pain-Function Responders
(N = 6 patients; bolded) were designated based on a delta value ≥10
and percentage change ≥20% in both KOOS pain and ADL scores
based on OMERACT-OARSI criteria [69]. Function-responders (N =
1; italics) and pain-responders (N = 1; italics) were classified
according to the same thresholds for patients responding in either
function (ADL) or pain scores, respectively. ADL: function in daily
living; KOOS: knee injury and osteoarthritis outcome score.

| | | | Pain | | ADL | |
|---|---|---|---|---|---|---|
| Response | Subject | Cell dose | | Percent | | Percent |
| classification | ID | (×10⁶ cells) | Delta | Change | Delta | Change |
| Non-responder | 1 | 1 | 5.50 | 28.35 | 2.90 | 32.95 |
| Non-responder | 2 | 1 | 11.10 | 18.17 | −5.90 | −25.11 |
| Function-Pain | 3 | 1 | 16.60 | 49.85 | 25.00 | 80.91 |
| Responder | | | | | | |
| Non-responder | 4 | 10 | −2.70 | −48.21 | 0.00 | 0.00 |
| Function-Pain | 5 | 10 | 13.90 | 29.45 | 11.80 | 100.00 |
| Responder | | | | | | |
| Non-responder/ | 6 | 10 | 16.70 | 33.40 | −8.80 | −200.00 |
| Pain responder | | | | | | |
| Function-Pain | 7 | 50 | 30.60 | 100.00 | 20.60 | 100.00 |
| Responder | | | | | | |
| Function-Pain | 9 | 50 | 22.20 | 31.99 | 19.10 | 43.31 |
| Responder | | | | | | |
| Non-responder/ | 10 | 50 | 11.10 | 19.04 | 17.70 | 42.96 |
| Function responder | | | | | | |
| Function-Pain | 11 | 50 | 36.10 | 81.31 | 20.60 | 70.07 |
| Responder | | | | | | |
| Non-responder | 12 | 10 | −30.50 | −78.41 | −14.70 | −45.37 |
| Function-Pain | 13 | 1 | 38.90 | 63.67 | 39.70 | 77.09 |
| Responder | | | | | | |

TABLE 15
Correlations between putative MSC(M) CQA genes and changes in KOOS
(percent change or delta values) at 12 and 24 months (test dataset).
N = 9 patients. Significant correlations (*p < 0.05) are
bolded. Spearman's correlation. ADL: function in daily living; Sports/Rec:
function in sport and recreation; QOL: knee-related quality of life;
KOOS: knee injury and osteoarthritis outcome score.

| | | | 12 Month | | 24 Month | |
|---|---|---|---|---|---|---|
| Gene/ | Follow-up | Difference | Spearman | | Spearman | |
| PC Score | Score | calculation | rho | p value | rho | p value |
| ACTA2 | Symptom | Percent change | −0.3000 | 0.4328 | −0.3667 | 0.3317 |
| | | Delta | −0.0924 | 0.8130 | −0.0667 | 0.8647 |
| | Pain | Percent change | 0.0333 | 0.9322 | −0.0333 | 0.9322 |
| | | Delta | 0.0833 | 0.8312 | −0.0167 | 0.9661 |
| | ADL | Percent change | −0.1506 | 0.6989 | −0.2833 | 0.4600 |
| | | Delta | −0.1925 | 0.6198 | −0.2000 | 0.6059 |
| | Sports/Rec | Percent change | −0.2667 | 0.4879 | 0.2594 | 0.5003 |
| | | Delta | −0.0783 | 0.8412 | 0.1849 | 0.6339 |
| | QOL | Percent change | −0.1500 | 0.7001 | −0.5021 | 0.1684 |
| | | Delta | 0.2594 | 0.5003 | −0.2639 | 0.4926 |
| | Overall | Percent change | −0.2667 | 0.4879 | −0.2833 | 0.4600 |
| | | Delta | 0.0000 | 1.0000 | −0.2176 | 0.5739 |
| ANGPT1 | Symptom | Percent change | −0.6000 | 0.0876 | −0.5500 | 0.1250 |
| | | Delta | −0.5630 | 0.1145 | −0.5000 | 0.1705 |
| | Pain | Percent change | −0.8333 | *0.0053 | −0.6500 | 0.0581 |
| | | Delta | −0.7833 | *0.0125 | −0.5833 | 0.0992 |
| | ADL | Percent change | −0.5523 | 0.1231 | −0.6833 | *0.0424 |
| | | Delta | −0.6862 | *0.0412 | −0.7000 | *0.0358 |
| | Sports/Rec | Percent change | −0.5333 | 0.1392 | −0.5105 | 0.1603 |
| | | Delta | −0.7311 | *0.0252 | −0.4874 | 0.1832 |
| | QOL | Percent change | −0.6833 | *0.0424 | −0.2510 | 0.5147 |
| | | Delta | −0.5941 | 0.0916 | −0.3916 | 0.2973 |
| | Overall | Percent change | −0.7333 | *0.0246 | −0.5833 | 0.0992 |
| | | Delta | −0.7167 | *0.0298 | −0.5941 | 0.0916 |
| CCN2 | Symptom | Percent change | 0.6667 | *0.0499 | 0.6500 | 0.0581 |
| | | Delta | 0.6723 | *0.0473 | 0.5500 | 0.1250 |
| | Pain | Percent change | 0.6167 | 0.0769 | 0.5667 | 0.1116 |
| | | Delta | 0.7000 | *0.0358 | 0.5167 | 0.1544 |
| | ADL | Percent change | 0.5439 | 0.1301 | 0.5833 | 0.0992 |
| | | Delta | 0.5356 | 0.1373 | 0.5500 | 0.1250 |
| | Sports/Rec | Percent change | 0.4500 | 0.2242 | 0.1506 | 0.6989 |
| | | Delta | 0.6615 | 0.0523 | 0.1092 | 0.7796 |
| | QOL | Percent change | 0.6667 | *0.0499 | 0.4435 | 0.2318 |
| | | Delta | 0.5607 | 0.1163 | 0.5022 | 0.1683 |
| | Overall | Percent change | 0.5500 | 0.1250 | 0.5667 | 0.1116 |
| | | Delta | 0.6667 | *0.0499 | 0.5774 | 0.1035 |
| CXCL8 | Symptom | Percent change | −0.2667 | 0.4879 | −0.3000 | 0.4328 |
| | | Delta | −0.4622 | 0.2103 | −0.3000 | 0.4328 |
| | Pain | Percent change | 0.0667 | 0.8647 | −0.2333 | 0.5457 |
| | | Delta | −0.1167 | 0.7650 | −0.2000 | 0.6059 |
| | ADL | Percent change | −0.3849 | 0.3063 | −0.2167 | 0.5755 |
| | | Delta | −0.1423 | 0.7150 | −0.1167 | 0.7650 |
| | Sports/Rec | Percent change | −0.1000 | 0.7980 | 0.0335 | 0.9319 |
| | | Delta | −0.2611 | 0.4974 | 0.0588 | 0.8805 |
| | QOL | Percent change | −0.2167 | 0.5755 | −0.3013 | 0.4308 |
| | | Delta | −0.3264 | 0.3914 | −0.3916 | 0.2973 |
| | Overall | Percent change | −0.0667 | 0.8647 | −0.2500 | 0.5165 |
| | | Delta | −0.2833 | 0.4600 | −0.2594 | 0.5003 |
| EDN1 | Symptom | Percent change | −0.4167 | 0.2646 | −0.5000 | 0.1705 |
| | | Delta | −0.6471 | 0.0596 | −0.5000 | 0.1705 |
| | Pain | Percent change | −0.2000 | 0.6059 | −0.5667 | 0.1116 |
| | | Delta | −0.3667 | 0.3317 | −0.4833 | 0.1875 |
| | ADL | Percent change | −0.4268 | 0.2520 | −0.4000 | 0.2861 |
| | | Delta | −0.4519 | 0.2220 | −0.4000 | 0.2861 |
| | Sports/Rec | Percent change | −0.2500 | 0.5165 | −0.4519 | 0.2220 |
| | | Delta | −0.4787 | 0.1923 | −0.5210 | 0.1503 |
| | QOL | Percent change | −0.3333 | 0.3807 | −0.4603 | 0.2125 |
| | | Delta | −0.5021 | 0.1684 | −0.6384 | 0.0642 |
| | Overall | Percent change | −0.3000 | 0.4328 | −0.5833 | 0.0992 |
| | | Delta | −0.5333 | 0.1392 | −0.5941 | 0.0916 |
| ICAM1 | Symptom | Percent change | −0.3667 | 0.3317 | −0.4833 | 0.1875 |
| | | Delta | −0.4874 | 0.1832 | −0.4000 | 0.2861 |
| | Pain | Percent change | −0.1500 | 0.7001 | −0.4833 | 0.1875 |
| | | Delta | −0.3333 | 0.3807 | −0.4167 | 0.2646 |
| | ADL | Percent change | −0.1172 | 0.7640 | −0.2333 | 0.5457 |
| | | Delta | −0.3264 | 0.3914 | −0.2833 | 0.4600 |
| | Sports/Rec | Percent change | 0.0500 | 0.8984 | −0.4017 | 0.2839 |
| | | Delta | −0.2524 | 0.5123 | −0.4454 | 0.2296 |
| | QOL | Percent change | −0.0833 | 0.8312 | −0.3180 | 0.4043 |
| | | Delta | −0.2594 | 0.5003 | −0.4086 | 0.2749 |
| | Overall | Percent change | −0.1333 | 0.7324 | −0.5000 | 0.1705 |
| | | Delta | −0.4000 | 0.2861 | −0.5188 | 0.1524 |
| PDCD1LG2 | Symptom | Percent change | 0.1500 | 0.7001 | −0.0833 | 0.8312 |
| | | Delta | 0.0000 | 1.0000 | −0.4167 | 0.2646 |
| | Pain | Percent change | 0.1000 | 0.7980 | −0.2167 | 0.5755 |
| | | Delta | −0.1167 | 0.7650 | −0.4167 | 0.2646 |
| | ADL | Percent change | −0.1255 | 0.7476 | −0.0833 | 0.8312 |
| | | Delta | −0.3013 | 0.4308 | −0.3333 | 0.3807 |
| | Sports/Rec | Percent change | 0.2667 | 0.4879 | −0.2594 | 0.5003 |
| | | Delta | 0.0348 | 0.9291 | −0.2857 | 0.4561 |
| | QOL | Percent change | 0.2333 | 0.5457 | 0.1925 | 0.6198 |
| | | Delta | 0.0418 | 0.9149 | −0.0170 | 0.9653 |
| | Overall | Percent change | −0.0833 | 0.8312 | −0.1167 | 0.7650 |
| | | Delta | −0.1667 | 0.6682 | −0.1255 | 0.7476 |
| THBS1 | Symptom | Percent change | −0.2167 | 0.5755 | −0.3833 | 0.3085 |
| | | Delta | −0.3698 | 0.3274 | −0.5667 | 0.1116 |
| | Pain | Percent change | −0.0167 | 0.9661 | −0.3667 | 0.3317 |
| | | Delta | −0.3167 | 0.4064 | −0.5000 | 0.1705 |
| | ADL | Percent change | −0.3180 | 0.4043 | −0.2333 | 0.5457 |
| | | Delta | −0.3264 | 0.3914 | −0.3500 | 0.3558 |
| | Sports/Rec | Percent change | 0.2167 | 0.5755 | −0.0921 | 0.8138 |
| | | Delta | −0.1654 | 0.6707 | −0.0420 | 0.9145 |
| | QOL | Percent change | 0.0000 | 1.0000 | −0.0167 | 0.9659 |
| | | Delta | −0.2008 | 0.6044 | −0.1873 | 0.6295 |
| | Overall | Percent change | −0.1333 | 0.7324 | −0.2833 | 0.4600 |
| | | Delta | −0.3833 | 0.3085 | −0.3096 | 0.4175 |
| TNFAIP6 | Symptom | Percent change | −0.0833 | 0.8312 | −0.2167 | 0.5755 |
| | | Delta | −0.0756 | 0.8467 | −0.0667 | 0.8647 |
| | Pain | Percent change | −0.1000 | 0.7980 | 0.0167 | 0.9661 |
| | | Delta | −0.2167 | 0.5755 | 0.0333 | 0.9322 |
| | ADL | Percent change | 0.2762 | 0.4720 | 0.1333 | 0.7324 |
| | | Delta | 0.0502 | 0.8979 | 0.0333 | 0.9322 |
| | Sports/Rec | Percent change | −0.2000 | 0.6059 | 0.3096 | 0.4175 |
| | | Delta | −0.0609 | 0.8763 | 0.2521 | 0.5128 |
| | QOL | Percent change | −0.2167 | 0.5755 | −0.5439 | 0.1301 |
| | | Delta | 0.0586 | 0.8810 | −0.3490 | 0.3573 |
| | Overall | Percent change | 0.1333 | 0.7324 | −0.1333 | 0.7324 |
| | | Delta | −0.0167 | 0.9661 | −0.1172 | 0.7640 |
| PC1 Score | Symptom | Percent change | −0.7667 | *0.0159 | −0.7500 | *0.0199 |
| | | Delta | −0.8404 | *0.0046 | −0.6667 | *0.0499 |
| | Pain | Percent change | −0.5333 | 0.1392 | −0.7000 | *0.0358 |
| | | Delta | −0.6500 | 0.0581 | −0.6333 | 0.0671 |
| | ADL | Percent change | −0.7448 | *0.0213 | −0.7167 | *0.0298 |
| | | Delta | −0.6444 | 0.0610 | −0.6333 | 0.0671 |
| | Sports/Rec | Percent change | −0.4333 | 0.2440 | −0.2929 | 0.4444 |
| | | Delta | −0.7311 | *0.0252 | −0.2689 | 0.4841 |
| | QOL | Percent change | −0.6500 | 0.0581 | −0.4770 | 0.1942 |
| | | Delta | −0.6611 | 0.0525 | −0.6129 | 0.0793 |
| | Overall | Percent change | −0.6333 | 0.0671 | −0.7000 | *0.0358 |
| | | Delta | −0.7667 | *0.0159 | −0.7113 | *0.0317 |

TABLE 16
Discriminant analysis summary for MSC(M)-mediated in vitro MΦ polarization. Samples (count)
were assigned to MΦ alone (Solo), responder, or non-responder groups to maximize separation
based on multivariate gene expression profiles of each sample. The discriminant analysis reduced
all -ddCt values into canonical variables and samples are reclassified into predicted groups based
on their gene expression profiles. The fit of the discriminant analysis is described by the entropy
R² (values closer to 1 indicate better fit), the fraction of samples that are misclassified
into incorrect groups, and by statistical significance testing (Wilks' Lambda, Pillai's
Trace, Hotelling-Lawley, Roy's Max Root; *p < 0.05). “Misclass” is misclassified.

| | | | | | Prob > F | | | |
|---|---|---|---|---|---|---|---|---|
| Responder | | Number | Percent | Entropy | Wilks' | Pillai's | Hotelling- | Roy's Max |
| Criteria | Count | misclass | misclass | R2 | Lambda | Trace | Lawley | Root |
| Function- | 28 | 2 | 7.14 | 0.7627 | *0.0002 | *0.0019 | *0.0001 | *<0.0001 |
| Pain | | | | | | | | |
| Responder | | | | | | | | |
| Function | 28 | 1 | 3.57 | 0.9073 | *<0.0001 | *<0.0001 | *<0.0001 | *<0.0001 |
| Responder | | | | | | | | |
| Pain | 28 | 3 | 10.71 | 0.7052 | *0.0004 | *0.0033 | *0.0002 | *<0.0001 |
| Responder | | | | | | | | |

TABLE 17
Summary of top Spearman's rho correlations for each MSC(M)
CQA gene with changes in KOOS at 24 months relative to baseline
(test dataset). mRNA transcripts are sorted from lowest to highest
Spearman's rho correlation coefficient. Significant correlations
(*p < 0.05) are bolded. Follow-up score column indicates
which KOOS subscale resulted in the strongest correlation for
each MΦ marker and whether it was based on a delta or
percentage (%) change calculation relative to baseline. Min/Max
column indicates whether minimization or maximization functions
were assigned based on whether the correlation was positive
or negative. N = 9. Spearman's correlation. ADL: function
in daily living; QOL: knee-related quality of life; KOOS: knee
injury and osteoarthritis outcome score.

| Gene | Follow-up Score | Spearman's rho | p value | Min/Max |
|---|---|---|---|---|
| ANGPT1 | ADL-Delta | −0.7 | 0.0358 | Min |
| EDN1 | QOL-Delta | −0.6384 | 0.0642 | Min |
| THBS1 | Symptoms-Delta | −0.5667 | 0.1116 | Min |
| TNFAIP6 | QOL-% | −0.5439 | 0.1301 | Min |
| ACTA2 | QOL-% | −0.5021 | 0.1684 | Min |
| PDCD1LG2 | Symptoms-Delta | −0.4167 | 0.2646 | Min |
| CXCL8 | QOL-Delta | −0.3916 | 0.2973 | Min |
| CCN2 | Symptoms-% | 0.65 | 0.0581 | Max |

TABLE 18
Summary of top Spearman's rho correlations for each MSC(M)
CQA gene with changes in KOOS at 12 months relative to baseline
(test dataset). mRNA transcripts are sorted from lowest
to highest Spearman's rho correlation coefficient.

| | Follow-up Score | Spearman's | | |
|---|---|---|---|---|
| Gene | (clinical data from [8]) | rho | p value | Min/Max |
| ANGPT1 | Pain-% | −0.8333 | 0.0053 | Min |
| EDN1 | Symptoms-Delta | −0.6471 | 0.0596 | Min |
| CXCL8 | Symptoms-Delta | −0.4622 | 0.2103 | Min |
| THBS1 | Overall-Delta | −0.3833 | 0.3085 | Min |
| PDCD1LG2 | ADL-Delta | −0.3013 | 0.4308 | Min |
| ACTA2 | Symptoms-% | −0.3 | 0.4328 | Min |
| TNFAIP6 | ADL-% | 0.2762 | 0.472 | Max |
| CCN2 | Pain-Delta | 0.7 | 0.0358 | Max |

Significant correlations (*p < 0.05) are bolded.
Follow-up score column indicates which KOOS subscale resulted in the strongest correlation for each Mϕ marker and whether it was based on a delta or percentage (%) change calculation relative to baseline.
Min/Max column indicates whether minimization or maximization functions were assigned based on whether the correlation was positive or negative.
N = 9.
Spearman's correlation.
ADL: function in daily living; QOL: knee-related quality of life; KOOS: knee injury and osteoarthritis outcome score.

### REFERENCES

1. Pittenger M F, Discher D E, Peault B M, Phinney D G, Hare J M, Caplan A I. Mesenchymal stem cell perspective: cell biology to clinical progress. npj Regen Med. 4(1): 1-15. 2019;
2. Robb K P, Fitzgerald J C, Barry F, Viswanathan S. Mesenchymal stromal cell therapy: progress in manufacturing and assessments of potency. Cytotherapy. 21(3):289-306. 2019;
3. Krampera M, Le Blanc K. Mesenchymal stromal cells: Putative microenvironmental modulators become cell therapy. Cell Stem Cell. 28(10): 1708-25. 2021;
4. Galipeau J, Sensébé L. Mesenchymal Stromal Cells: Clinical Challenges and Therapeutic Opportunities. Cell Stem Cell. 22(6):824-33. 2018;
5. Weiss D J, English K, Krasnodembskaya A, Isaza-Correa J M, Hawthorne I J, Mahon B P. The necrobiology of mesenchymal stromal cells affects therapeutic efficacy. Front Immunol. 10:1228. 2019;
6. Liu S, De Castro L F, Jin P, Civini S, Ren J, Reems J A, et al. Manufacturing differences affect human bone marrow stromal cell characteristics and function: Comparison of production methods and products from multiple centers. Sci Rep. 7(1):1-11. 2017;
7. Czapla J, Matuszczak S, Kulik K, Wiśniewska E, Pilny E, Jarosz-Biej M, et al. The effect of culture media on large-scale expansion and characteristic of adipose tissue-derived mesenchymal stromal cells. Stem Cell Res Ther. 10(1):235. 2019;
8. Chahal J, Gómez-Aristizábal A, Shestopaloff K, Bhatt S, Chaboureau A, Fazio A, et al. Bone Marrow Mesenchymal Stromal Cells in Patients with Osteoarthritis Results in Overall Improvement in Pain and Symptoms and Reduces Synovial Inflammation. Stem Cells Transl Med. 8(8):746-757. 2019;
9. Galleu A, Riffo-Vasquez Y, Trento C, Lomas C, Dolcetti L, Cheung T S, et al. Apoptosis in mesenchymal stromal cells induces in vivo recipient-mediated immunomodulation. Sci Transl Med. 9(416)2017;
10. von Bahr L, Sundberg B, Lönnies L, Sander B, Karbach H, Hägglund H, et al. Long-term complications, immunologic effects, and role of passage for outcome in mesenchymal stromal cell therapy. Biol Blood Marrow Transplant. 18(4):557-64. 2012;
11. Chinnadurai R, Rajan D, Qayed M, Arafat D, Garcia M, Liu Y, et al. Potency Analysis of Mesenchymal Stromal Cells Using a Combinatorial Assay Matrix Approach. Cell Rep. 2018/03/01. 22(9):2504-17. 2018;
12. Lipat A J, Cottle C, Pirlot B M, Mitchell J, Pando B, Helmly B, et al. Chemokine Assay Matrix Defines the Potency of Human Bone Marrow Mesenchymal Stromal Cells. Stem Cells Transl Med. 2022;
13. Chinnadurai R, Rajakumar A, Schneider A J, Bushman W A, Hematti P, Galipeau J. Potency Analysis of Mesenchymal Stromal Cells Using a Phospho-STAT Matrix Loop Analytical Approach. Stem Cells. 37(8): 1119-25. 2019;
14. Maughon T S, Shen X, Huang D, Michael AOA, Shockey W A, Andrews S H, et al. Metabolomics and cytokine profiling of mesenchymal stromal cells identify markers predictive of T-cell suppression. Cytotherapy. 24(2): 137-48. 2022;
15. Klinker M W, Marklein R A, Lo Surdo J L, Wei C-H H, Bauer S R. Morphological features of IFN-gamma-stimulated mesenchymal stromal cells predict overall immunosuppressive capacity. Proc Natl Acad Sci USA. 2017/03/12. 114(13):E2598-e2607. 2017;
16. Andrews S H, Klinker M W, Bauer S R, Marklein R A. Morphological landscapes from high content imaging reveal cytokine priming strategies that enhance mesenchymal stromal cell immunosuppression. Biotechnol Bioeng. 119(2):361-75. 2022;
17. Boregowda S V., Krishnappa V, Haga C L, Ortiz L A, Phinney D G. A Clinical Indications Prediction Scale Based on TWIST1 for Human Mesenchymal Stem Cells. EBioMedicine. 2016/03/17. 4:62-73. 2016;
18. Oncologic drugs advisory committee. Remestemcel-L for treatment of steroid refractory acute graft versus host disease in pediatric patients [Internet]. FDA Advisory Committee. 2020.
19. Lehman N, Cutrone R, Raber A, Perry R, van't Hof W, Deans R, et al. Development of a surrogate angiogenic potency assay for clinical-grade stem cell production. Cytotherapy. 14(8):994-1004. 2012;
20. Kuznetsov S A, Mankani M H, Bianco P, Robey P G. Enumeration of the colony-forming units-fibroblast from mouse and human bone marrow in normal and pathological conditions. Stem Cell Res. 2(1):83-94. 2009;
21. Wang J, Liao L, Wang S, Tan J. Cell therapy with autologous mesenchymal stem cells-how the disease process impacts clinical considerations. Cytotherapy. 15(8):893-904. 2013;
22. Siegel G, Kluba T, Hermanutz-Klein U, Bieback K, Northoff H, Schäfer R. Phenotype, donor age and gender affect function of human bone marrow-derived mesenchymal stromal cells. BMC Med. 11(1):146. 2013;
23. Yin J Q, Zhu J, Ankrum J A. Manufacturing of primed mesenchymal stromal cells for therapy. Nat Biomed Eng. 3(2):90-104. 2019;
24. Wilson A, Hodgson-Garms M, Frith J E, Genever P. Multiplicity of mesenchymal stromal cells: Finding the right route to therapy. Front Immunol. 10:1112. 2019;
25. Huang Y, Li Q, Zhang K, Hu M, Wang Y, Du L, et al. Single cell transcriptomic analysis of human mesenchymal stem cells reveals limited heterogeneity. Cell Death Dis. 10(5): 1-12. 2019;
26. Wiese D M, Wood C A, Ford B N, Braid L R. Cytokine activation reveals tissue-imprinted gene profiles of mesenchymal stromal cells. Front Immunol. 2022;
27. Lee J H, Yoon Y M, Lee S H. Hypoxic preconditioning promotes the bioactivities of mesenchymal stem cells via the HIF-1α-GRP78-Akt axis. Int J Mol Sci. 18(6)2017;
28. Leroux L, Descamps B, Tojais N F, Seguy B, Oses P, Moreau C. Hypoxia preconditioned mesenchymal stem cells improve vascular and skeletal muscle fiber regeneration after ischemia through a Wnt4-dependent pathway. Mol Ther. 182010;
29. Wobma H M, Kanai M, Ma S P, Shih Y, Li H W, Duran-Struuck R, et al. Dual IFN-γ/hypoxia priming enhances immunosuppression of mesenchymal stromal cells through regulatory proteins and metabolic mechanisms. J Immunol Regen Med. 1:45-56. 2018;
30. Kadle R L, Abdou S A, Villarreal-Ponce A P, Soares M A, Sultan D L, David J A, et al. Microenvironmental cues enhance mesenchymal stem cell-mediated immunomodulation and regulatory T-cell expansion. PLOS One. 2018/03/08. 13:e0193178. 2018;
31. Bartosh T J, Ylostalo J H, Mohammadipoor A, Bazhanov N, Coble K, Claypool K, et al. Aggregation of human mesenchymal stromal cells (MSCs) into 3D spheroids enhances their antiinflammatory properties. Proc Natl Acad Sci USA. 2010/07/21. 107(31): 13724-9. 2010;
32. Miceli V, Pampalone M, Vella S, Carreca A P, Amico G, Conaldi P G. Comparison of Immunosuppressive and Angiogenic Properties of Human Amnion-Derived Mesenchymal Stem Cells Between 2D and 3D Culture Systems. Stem Cells Int. 2019;
33. Miranda J P, Camões S P, Gaspar M M, Rodrigues J S, Carvalheiro M, Bárcia R N, et al. The Secretome Derived From 3D-Cultured Umbilical Cord Tissue MSCs Counteracts Manifestations Typifying Rheumatoid Arthritis. Front Immunol. 10:18. 2019;
34. Cheng N-C, Chen S-Y, Li J-R, Young T-H. Short-Term Spheroid Formation Enhances the Regenerative Capacity of Adipose-Derived Stem Cells by Promoting Stemness, Angiogenesis, and Chemotaxis. Stem Cells Transl Med. 2(8):584-94. 2013;
35. Potapova I A, Gaudette G R, Brink P R, Robinson R B, Rosen M R, Cohen I S, et al. Mesenchymal Stem Cells Support Migration, Extracellular Matrix Invasion, Proliferation, and Survival of Endothelial Cells In Vitro. Stem Cells. 25(7): 1761-8. 2007;
36. Zimmermann J A, Mcdevitt T C. Pre-conditioning mesenchymal stromal cell spheroids for immunomodulatory paracrine factor secretion. Cytotherapy. 16(3):331-45. 2014;
37. Galipeau J. Macrophages at the nexus of mesenchymal stromal cell potency: The emerging role of chemokine cooperativity. Stem Cells. 2021;
38. Thej C, Ramadasse B, Walvekar A, Majumdar A S, Balasubramanian S. Development of a surrogate potency assay to determine the angiogenic activity of Stempeucel®, a pooled, ex-vivo expanded, allogeneic human bone marrow mesenchymal stromal cell product. Stem Cell Res Ther. 2017/03/02. 8(1):47. 2017;
39. Viswanathan S, Bhatt S J, Gomez-Aristizabal A. Methods of culturing mesenchymal stromal cells. World Intellectual Property Organization; WO2018053618A1, 2017.
40. Egger D, Schwedhelm I, Hansmann J, Kasper C. Hypoxic Three-Dimensional Scaffold-Free Aggregate Cultivation of Mesenchymal Stem Cells in a Stirred Tank Reactor. Bioeng. 2017/09/28. 42017;
41. Schneider C A, Rasband W S, Eliceiri K W. NIH Image to ImageJ: 25 years of image analysis. Nat Methods. 9(7):671-5. 2012;
42. Bourin P, Bunnell B A, Casteilla L, Dominici M, Katz A J, March K L, et al. Stromal cells from the adipose tissue-derived stromal vascular fraction and culture expanded adipose tissue-derived stromal/stem cells: a joint statement of the International Federation for Adipose Therapeutics and Science (IFATS) and the International So. Cytotherapy. 15(6):641-8. 2013;
43. Edgar R, Domrachev M, Lash A E. Gene Expression Omnibus: NCBI gene expression and hybridization array data repository. Nucleic Acids Res. 30(1):207-10. 2002;
44. Gómez-Aristizábal A, Kim K-P P, Viswanathan S. A Systematic Study of the Effect of Different Molecular Weights of Hyaluronic Acid on Mesenchymal Stromal Cell-Mediated Immunomodulation. PLOS One. 11(1):e0147868. 2016;
45. Carpentier G. Angiogenesis Analyzer for ImageJ [Internet]. ImageJ News. 2012.
46. Benjamini Y, Yekutieli D. The control of the false discovery rate in multiple testing under dependency. Ann Stat. 29(4): 1165-88. 2001;
47. Derringer G, Suich R. Simultaneous Optimization of Several Response Variables. J Qual Technol. 12(4):214-9. 1980;
48. Wobma H M, Tamargo M A, Goeta S, Brown L M, Duran-Struuck R, Vunjak-Novakovic G. The influence of hypoxia and IFN-γ on the proteome and metabolome of therapeutic mesenchymal stem cells. Biomaterials. 167:226-34. 2018;
49. Watt S M, Gullo F, van der Garde M, Markeson D, Camicia R, Khoo C P, et al. The angiogenic properties of mesenchymal stem/stromal cells and their therapeutic potential. Br Med Bull. 108:25-53. 2013;
50. Ren G, Su J, Zhang L, Zhao X, Ling W, L'huillie A, et al. Species Variation in the Mechanisms of Mesenchymal Stem Cell-Mediated Immunosuppression. Stem Cells. 27(8): 1954-62. 2009;
51. Sekiya I, Katano H, Ozeki N. Characteristics of mscs in synovial fluid and mode of action of intra-articular injections of synovial mscs in knee osteoarthritis. Int J Mol Sci. 22(6): 1-13. 2021;
52. Ozeki N, Muneta T, Koga H, Nakagawa Y, Mizuno M, Tsuji K, et al. Not single but periodic injections of synovial mesenchymal stem cells maintain viable cells in knees and inhibit osteoarthritis progression in rats. Osteoarthr Cartil. 24(6): 1061-70. 2016;
53. Ghazanfari R, Zacharaki Di, Li H, Ching Lim H, Soneji S, Scheding S. Human Primary Bone Marrow Mesenchymal Stromal Cells and Their in vitro Progenies Display Distinct Transcriptional Profile Signatures. Sci Rep. 7(1):1-10. 2017;
54. Arnaoutova I, George J, Kleinman H K, Benton G. The endothelial cell tube formation assay on basement membrane turns 20: State of the science and the art. Angiogenesis. 12(3):267-74. 2009;
55. Dominici M, Le Blanc K, Mueller I, Slaper-Cortenbach I, Marini F C, Krause D S, et al. Minimal criteria for defining multipotent mesenchymal stromal cells. The International Society for Cellular Therapy position statement. Cytotherapy. 8(4):315-7. 2006;
56. Mendicino M, Bailey A M, Wonnacott K, Puri R K, Bauer S R. MSC-based product characterization for clinical trials: an FDA perspective. Cell Stem Cell. 14:141-5. 2014;
57. Bhat S, Viswanathan P, Chandanala S, Prasanna S J, Seetharam R N. Expansion and characterization of bone marrow derived human mesenchymal stromal cells in serum-free conditions. Sci Rep. 11(1):3403. 2021;
58. Galipeau J, Krampera M, Barrett J, Dazzi F, Deans R J, DeBruijn J, et al. International Society for Cellular Therapy perspective on immune functional assays for mesenchymal stromal cells as potency release criterion for advanced phase clinical trials. Cytotherapy. 18:151-9. 2016;
59. Viswanathan S, Shi Y, Galipeau J, Krampera M, Leblanc K, Martin I, et al. Mesenchymal stem versus stromal cells: International Society for Cell & Gene Therapy (ISCT®) Mesenchymal Stromal Cell committee position statement on nomenclature. Cytotherapy. 21(10): 1019-24. 2019;
60. Ylostalo J H, Bartosh T J, Tiblow A, Prockop D J. Unique characteristics of human mesenchymal stromal/progenitor cells pre-activated in 3-dimensional cultures under different conditions. Cytotherapy. 2014/09/19. 16:1486-500. 2014;
61. Rabani R, Volchuk A, Jerkic M, Ormesher L, Garces-Ramirez L, Canton J, et al. Mesenchymal stem cells enhance NOX2-dependent reactive oxygen species production and bacterial killing in macrophages during sepsis. Eur Respir J. 51(4): 1702021. 2018;
62. Giri J, Das R, Nylen E, Chinnadurai R, Galipeau J. CCL2 and CXCL12 Derived from Mesenchymal Stromal Cells Cooperatively Polarize IL-10+ Tissue Macrophages to Mitigate Gut Injury. Cell Rep. 30(6): 1923-1934.e4. 2020;
63. Lee R H, Boregowda S V, Shigemoto-Kuroda T, Ortiz L A, Phinney D G. Mesenchymal Stem/Stromal Cells: TWIST1 and TSG6 as potency biomarkers of human MSCs in pre-clinical disease models. Cytotherapy. 24(5): S32. 2022;
64. Lee R H, Yu J M, Foskett A M, Peltier G, Reneau J C, Bazhanov N, et al. TSG-6 as a biomarker to predict efficacy of human mesenchymal stem/progenitor cells (hMSCs) in modulating sterile inflammation in vivo. Proc Natl Acad Sci USA. 111:16766-71. 2014;
65. Choi H, Lee R H, Bazhanov N, Oh J Y, Prockop D J. Anti-inflammatory protein TSG-6 secreted by activated MSCs attenuates zymosan-induced mouse peritonitis by decreasing TLR2/NF-κB signaling in resident macrophages. Blood. 118:330-8. 2011;
66. Galipeau J, Krampera M, Leblanc K, Nolta J A, Phinney D G, Shi Y, et al. Mesenchymal stromal cell variables influencing clinical potency: the impact of viability, fitness, route of administration and host predisposition. Cytotherapy. 23(5):368-72. 2021;
67. Belluzzi E, El Hadi H, Granzotto M, Rossato M, Ramonda R, Macchi V, et al. Systemic and Local Adipose Tissue in Knee Osteoarthritis. J Cell Physiol. 232(8): 1971-8. 2017;
68. Roos E M, Lohmander L S. The Knee injury and Osteoarthritis Outcome Score (KOOS): From joint injury to osteoarthritis. Health Qual Life Outcomes 2003; 1. https://doi.org/10.1186/1477-7525-1-64.
69. Pham T, van der Heijde D, Altman R D, Anderson J J, Bellamy N, Hochberg M, et al. OMERACT-OARSI initiative: Osteoarthritis research society international set of responder criteria for osteoarthritis clinical trials revisited. Osteoarthritis Cartilage 2004; 12:389-99. https://doi.org/10.1016/j.joca.2004.02.001.
70. Chen H H, Chen Y C, Yu S N, Lai W L, Shen Y S, Shen P C, et al. Infrapatellar fat pad-derived mesenchymal stromal cell product for treatment of knee osteoarthritis: a first-in-human study with evaluation of the potency marker. Cytotherapy 2022; 24:72-85. https://doi.org/10.1016/j.jcyt.2021.08.006.
71. Kabat M, Bobkov I, Kumar S, Grumet M. Trends in mesenchymal stem cell clinical trials 2004-2018: Is efficacy optimal in a narrow dose range? Stem Cells Transl Med 2020; 9:17-27. https://doi.org/10.1002/SC™.19-0202.
72. Copp G, Robb K P, Viswanathan S. Culture-expanded mesenchymal stromal cell therapy: does it work in knee osteoarthritis? A pathway to clinical success. Cell Mol Immunol 2023. https://doi.org/10.1038/S41423-023-01020-1.

1. A method of determining immunomodulatory fitness of mesenchymal stromal cells (MSC) comprising:
determining a sample critical quality attribute (CQA) profile, the sample CQA profile comprising:
a) from the sample harvested:
a. in the presence of proinflammatory cytokines stimulation post-harvest: the mRNA levels of the following genes: THBS1, CCN2, EDN1, ACTA2, PDCD1LG2, TNFAIP6, ANGPT1, CXCL8; and/or
b. in the absence of the proinflammatory cytokines stimulation post-harvest: the mRNA levels of the following genes: CCN2, TSG101, THBS1, PDGFA, VEGFA, EDIL3, ACTA2, ANGPT1, ANG and the level of TGF-beta protein; and/or
b) from a single cell suspension of the sample post-harvest without pro-inflammatory cytokines stimulation: cell diameter and cell circularity;
determining the level of similarity of said sample CQA profile to one or more control profiles, wherein (i) a high level of similarity of the sample CQA profile to an immunomodulatory specific control profile; (ii) a low level of similarity to a non-immunomodulatory specific control profile; and/or (iii) a higher level of similarity to an immunomodulatory specific control profile than to a non-immunomodulatory specific control profile indicates the cells have basal immunomodulatory fitness.

2. A method of determining angiogenic fitness of derived mesenchymal stromal cells (MSC) comprising:
determining a sample critical quality attribute (CQA) profile, the sample CQA profile comprising:
a) from the sample harvested and cultured:
a. in the presence of proinflammatory cytokines stimulation post-harvest: the mRNA levels of the following genes: SMAD7, HGF, ANG, IDO1, TSG101, CXCL8 and the level of VEGF protein; and/or
b. in the absence of the proinflammatory cytokines stimulation post-harvest: the mRNA levels of the following genes: SOX9, TNFAIP6 and the level of VEGF protein;
determining the level of similarity of said sample CQA profile to one or more control profiles, wherein (i) a high level of similarity of the sample CQA profile to an angiogenic specific control profile; (ii) a low level of similarity to a non-angiogenic specific control profile; and/or (iii) a higher level of similarity to an angiogenic specific control profile than to a non-angiogenic specific control profile indicates the cells have basal angiogenic fitness.

3. The method of claim 1, wherein a higher level of similarity to the immunomodulatory specific control profile than to the non-immunomodulatory specific control profile is indicated by a higher correlation value computed between the sample profile and the immunomodulatory specific control profile than an equivalent correlation value computed between the sample profile and the non-immunomodulatory specific control profile, optionally wherein the correlation value is a correlation coefficient.

4. The method of claim 2, wherein a higher level of similarity to the angiogenic specific control profile than to the non-angiogenic specific control profile is indicated by a higher correlation value computed between the sample profile and the angiogenic specific control profile than an equivalent correlation value computed between the sample profile and the non-angiogenic specific control profile, optionally wherein the correlation value is a correlation coefficient.

5. A method of determining quantitative values for a set of CQAs for assessing immunomodulatory fitness of samples of mesenchymal stromal cells (MSC), wherein the MSC samples are from donors and/or from samples grown using different critical processing parameters (CPPs), the method comprising:
a) determining the values of the set of CQAs for each sample; wherein the set of CQAs comprise:
a. from the sample cultured and harvested:
i. in the presence of proinflammatory cytokines stimulation post-harvest: the mRNA levels of the following genes: THBS1, CCN2, EDN1, ACTA2, PDCD1LG2, TNFAIP6, ANGPT1, CXCL8; and/or
ii. in the absence of the proinflammatory cytokines stimulation post-harvest: the mRNA levels of the following genes: CCN2, TSG101, THBS1, PDGFA, VEGFA, EDIL3, ACTA2, ANGPT1, ANG and the level of TGF-beta protein; and/or
b. from a single cell suspension of the sample: cell diameter and cell circularity post-harvest without pro-inflammatory cytokine stimulation;
b) testing each sample of MSC for immunomodulatory fitness using in vitro assays or obtaining immunomodulatory fitness status of the sample;
c) conducting a regression analysis between each CQA of a) with the immunomodulatory fitness of b); determining minimum and maximum data values of each CQA of the samples and using multivariate dimension reduction statistical or mathematical methods to assign minimization or maximization functions to indicate whether higher or lower values are desirable based on the directionality of correlation respectively.

6. The method of claim 5, further comprising
d) assigning a weighting to each CQA for desirability analysis based on the R2 values from the regression analysis; and
e) use of desirability analysis methods to assign a score from zero to one to each sample based on the range of data values; wherein zero is undesirable and one is highly desirable to obtain a set of values for MSCs for each donor grown using particular CPPs.

7. The method of claim 5, wherein the testing for immunomodulatory fitness in step b);
i) comprises measuring in vitro MSC-mediated polarization of monocyte/macrophages toward inflammation-resolving phenotypes, regulatory T cell (Treg) induction, suppression of peripheral blood mononuclear cells (PBMC) or T cell proliferation;
ii) comprises evaluating immunomodulatory effects of MSC using preclinical animal models;
iii) is obtained from a clinical sample for which the immunomodulatory fitness or efficacy of the MSCs were previously evaluated clinically; or
iv) comprises:
I. determining at least one patient-reported outcome measure (PROM) score from an assessment of an MSC-treatable disease from a subject with the MSC-treatable disease pre treatment;
II. determining the at least one PROM score from the assessment of the MSC-treatable disease from the subject post treatment;
III. comparing the at least one PROM score in I) and II); and
IV. assigning an immunomodulatory fitness score to the sample based on the size of the improvement in the at least one PROM score post-treatment compared to pre-treatment, wherein an improvement in the at least one PROM score post-treatment is indicative that the immunomodulatory fitness score of the sample is high, and wherein no improvement or a worsening in the at least one PROM score post-treatment is indicative that the immunomodulatory fitness score of the sample is low.

8. The method of claim 5, wherein the method of a) comprises a.i. and/or a.ii.

9. The method of claim 7, wherein step iv) II. is performed 12 or 24 months post treatment with the sample.

10. The method of claim 5, wherein the MSC-treatable disease comprises osteoarthritis, lupus, scleroderma, rheumatoid arthritis, graft versus host disease, stroke, inflammatory bowel disease, or cardiac disease.

11. The method of claim 10, wherein the osteoarthritis comprises knee osteoarthritis, and wherein the assessment of the MSC-treatable disease comprises an assessment of knee osteoarthritis.

12. The method of claim 11, wherein the assessment of knee osteoarthritis comprises Knee injury and Osteoarthritis Outcome Score (KOOS), Western Ontario and McMaster Universities Osteoarthritis Index (WOMAC), Visual Analogue Scale (VAS), Short Form 36 (SF-36) or Short Form 12 (SF-12), Tegner Lysholm Knee Score, Knee Society Clinical Rating System, Lequesne Index for Knee Osteoarthritis, Oxford Knee Score (OKS) and/or International Knee Documentation Committee (IKDC) Questionnaire.

13. A method of determining immunomodulatory fitness of mesenchymal stromal cells (MSC) comprising
a) determining a sample critical quality attribute (CQA) profile, the sample CQA profile comprising:
a. from the sample harvested in the presence of proinflammatory cytokines stimulation post-harvest: the mRNA levels of the following genes: THBS1, CCN2, EDN1, ACTA2, PDCD1LG2, TNFAIP6, ANGPT1, CXCL8; and/or
b. from the sample harvested in the absence of the proinflammatory cytokines stimulation post-harvest: the mRNA levels of the following genes: CCN2, TSG101, THBS1, PDGFA, VEGFA, EDIL3, ACTA2, ANGPT1, ANG and the level of TGF-beta protein; and/or
c. from a single cell suspension of the sample post-harvest without pro-inflammatory cytokines stimulation: cell diameter and cell circularity; and
b) ranking the CQA profile of a) based on a comparison of minimum and maximum values determined by the method of claim 5 for the same particular donor using the same particular CPPs to determine the immunomodulatory fitness.

14. A method of determining quantitative values for a set of CQAs for assessing angiogenic fitness of samples of mesenchymal stromal cells (MSC), wherein the MSC samples are from different donors and/or from samples grown using different critical processing parameters (CPPs), the method comprising:
a) determining the values of the set of CQAs for each sample; wherein the set of CQAs comprise:
a. from the sample cultured and harvested:
i. in the presence of proinflammatory cytokines post-harvest stimulation: the mRNA levels of the following genes: SMAD7, HGF, ANG, IDO1, TSG101, CXCL8 and the level of VEGF protein; and/or
ii. in the absence of the proinflammatory cytokines post-harvest stimulation: the mRNA levels of the following genes: SOX9, TNFAIP6 and the level of VEGF protein; and
b) testing each sample of MSCs for angiogenic fitness using in vitro assays or obtaining angiogenic fitness status of the sample;
c) conducting a regression analysis between each sample CQA of a) with the angiogenic fitness of b); determining minimum and maximum data values of each CQA of the samples and using multivariate dimension reduction statistical or mathematical methods to assign minimization or maximization functions to indicate whether higher or lower values are desirable based on the directionality correlation respectively.

15. The method of claim 14, further comprising
d) assigning a weighting to each CQA for desirability analysis based on the R2 values from the regression analysis; and
e) use of desirability analysis methods to assign a score from zero to one to each sample based on the range of data values; wherein zero is undesirable and one is highly desirable to obtain a set of values for MSCs for each donor grown using particular CPPs.

16. The method of claim 14, wherein the testing for angiogenic fitness in step b):
i) comprises an in vitro human umbilical vein tube formation assay, measurement of MSC-mediated effects on endothelial cells, assays for vessel outgrowth or the chick chorioallantoic membrane assay;
ii) comprises evaluating angiogenic effects of MSC using preclinical animal models; or
iii) is obtained from a clinical sample for which the angiogenic fitness or efficacy of the MSCs were previously evaluated clinically.

17. A method of determining angiogenic fitness of mesenchymal stromal cells (MSC) from a particular donor using particular CPPs comprising
a) determining a sample critical quality attribute (CQA) profile, the sample CQA profile comprising:
a. from the sample harvested in the presence of proinflammatory cytokines stimulation post-harvest: the mRNA levels of the following genes: SMAD7, HGF, ANG, IDO1, TSG101, CXCL8 and the level of VEGF protein; and/or
b. from the sample harvested in the absence of the proinflammatory cytokines stimulation post-harvest: the mRNA levels of the following genes: SOX9, TNFAIP6 and the level of VEGF protein;
b) ranking the CQA profile of a) based on a comparison of minimum and maximum values determined by the method of claim 14 for the same particular donor using the same particular CPPs to determine the angiogenic fitness.

18. The method of claim 5, wherein the MSCs are derived from adipose tissue, bone marrow, dental pulp, synovium, placenta, or umbilical cord.

19. The method of claim 18, wherein the MSCs are derived from adipose tissue or from bone marrow.

20. The method of claim 14, wherein the MSCs are derived from adipose tissue, bone marrow, dental pulp, synovium, placenta, or umbilical cord.

21. The method of claim 5, wherein the proinflammatory cytokines stimulation post-harvest comprise IFNγ, TNFα and/or IL-1β.

22. The method of claim 14, wherein the proinflammatory cytokines stimulation post-harvest comprise IFNγ, TNFα and/or IL-1β.
